(19) United States
(12) Patent Application Publication
(10) Pub. No.: US 2023/0181493 A1
(43) Pub. Date: Jun. 15, 2023

(54) BIOMARKERS RELATED TO PARKINSON'S DISEASE AND METHODS OF USING THE SAME

(71) Applicant: SENDA BIOSCIENCES, INC., Cambridge, MA (US)

(72) Inventors: Devin Forest Reed DOUD, Arlington, MA (US); Afrand KAMALI, Somerville, MA (US); Victoria CATANZANO, Tewksbury, MA (US); Yohannes TEFFERA, Topsfield, MA (US); Spencer Cory PECK, Watertown, MA (US); William McELROY, Cambridge, MA (US); Timothy F. BRIGGS, Waltham, MA (US); Steven John TAYLOR, Winchester, MA (US)

(21) Appl. No.: 17/995,749

(22) PCT Filed: Apr. 9, 2021
(86) PCT No.: PCT/US2021/026630

§ 371 Date: Oct. 7, 2022

Related U.S. Application Data
(60) Provisional application No. 63/008,121, filed on Apr. 10, 2020.

(51) Int. Cl. A61K 31/137; G01N 33/68; A61P 25/16

(52) U.S. Cl. CPC A61K 31/137; G01N 33/6896; A61P 25/16; G01N 2800/52

(57) ABSTRACT

The present disclosure relates to the treatment of Parkinson's disease. The present disclosure provides, in some embodiments, methods of treating Parkinson's disease in a patient in need thereof. In some embodiments, the methods disclosed herein comprise administering a levodopa therapy based on a patient's biomarker profile. In some embodiments, the levodopa therapy comprises or lacks a tyrosine decarboxylase inhibitor. Therapeutic uses and compositions are also disclosed.

This application claims the benefit of priority to U.S. Provisional Patent Application No. 63/008,121, filed Apr. 10, 2020, which is incorporated herein by reference in its entirety.

The present disclosure relates to the treatment of Parkinson's disease. The present disclosure provides, in some embodiments, methods of treating Parkinson's disease in a patient in need thereof. In some embodiments, the methods disclosed herein comprise administering a levodopa therapy based on a patient's biomarker profile. In some embodiments, the levodopa therapy comprises or lacks a tyrosine decarboxylase inhibitor. In some embodiments, the biomarker profile comprises one or more biomarkers that indicate the presence and/or extent of microbial metabolism of levodopa in the patient. In some embodiments, the biomarker profile comprises meta-tyramine or a metabolic derivative thereof. In some embodiments, the methods disclosed herein comprise administering to a Parkinson's disease patient having an elevated level of microbial metabolism of levodopa, as determined from one or more biomarkers described herein (e.g., meta-tyramine or a metabolic derivative thereof), a levodopa therapy comprising a tyrosine decarboxylase inhibitor. In some embodiments, the methods disclosed herein comprise administering to a Parkinson's disease patient having a normal or low level of microbial metabolism of levodopa, as determined from one or more biomarkers described herein (e.g., meta-tyramine or a metabolic derivative thereof), a levodopa therapy lacking a tyrosine decarboxylase inhibitor. Therapeutic uses and compositions are also provided.

The bacterial communities inhabiting the mammalian gut can impact the health of their host (Kahrstrom et al., Nature. 2016; 535:47). Numerous reports indicate that intestinal microbiota and metabolic products thereof can affect various health and disease states. Host immune system and brain development, metabolism, behavior, stress, and pain response have all been reported to be associated with microbiota disturbances (Yano et al., Cell. 2015; 161:264-276; Mao et al., Nature. 2018; 554:255-259; Pusceddu et al., PLoS ONE. 2015; 10:e0139721; El Aidy et al., Mucosal Immunol. 2012; 5:567-579; Kelly et al., J. Psychiatr. Res. 2016; 82:109-118). It is also becoming increasingly clear that gut microbiota can interfere with the modulation of drug efficacy (Enright et al., Yale J. Biol. Med. 2016; 89:375-382; Niehues et al., J. Pharm. Pharmacol. 2009; 61:1303-1307).

Parkinson's disease (PD), the second most common neurodegenerative disease after Alzheimer's, is estimated to affect about 1% of the global population over the age of 60 (Bekris et al., J Geriatr Psychiatry Neurol. 2010; 23:228-242), and has been correlated with alterations in microbial gut composition (Pereira et al., Park. Relat. Disord. 2017; 38:61-67; Sampson et al., 2016; 167:1469-1480; Scheperjans et al., Mov. Disord. 2014; 30:350-358).

Levodopa (L-3,4-dihydroxyphenylalanine), a dopamine precursor, is commonly used in combination with an aromatic amino acid decarboxylase inhibitor (such as carbidopa) to treat symptoms of Parkinson's disease (Deleu et al., Clin. Pharmacokinet. 2002; 41:261-309). However, the bioavailability of the levodopa and decarboxylase inhibitor required to ensure that sufficient amounts of dopamine reach the brain varies significantly among Parkinson's disease patients (Pinder, Nature. 1970; 228:358). Levodopa/decarboxylase inhibitor combinations are ineffective in a subset of patients, and in other patients, efficacy decreases over the treatment period, necessitating more frequent drug doses and increasing the risk of dyskinesia and other undesirable side effects (Katzenschlager et al., J. Neurol. 2002; 249(Suppl 2):li19-li24).

Several amino acid decarboxylases have been identified in bacteria. Tyrosine decarboxylase (TDC) genes (tdc) are encoded in the genome of several bacterial species in the genera Lactobacillus and Enterococcus (Perez et al., Appl. Microbiol. Biotechnol. 2015; 99:3547-3558; Zhu et al., Sci. Rep. 2016; 6:27779). Though TDC is named for its capacity to decarboxylate L-tyrosine into tyramine, recent studies have demonstrated that bacterial tyrosine decarboxylases can efficiently convert levodopa to dopamine (van Kessel et al., Nat. Commun. 2019; 10(1):310; Rekdal et al., Science 2019; 364(6445):eaau6323). It has also been reported that in situ levels of levodopa are compromised by a high abundance of gut bacterial tyrosine decarboxylase in patients with Parkinson's disease, and that a higher relative abundance of bacterial tyrosine decarboxylases at the site of levodopa absorption, the proximal small intestine, decreases levels of levodopa in the plasma of rats (van Kessel et al., Nat. Commun. 2019; 10(1):310). These observations suggest that microbial metabolism affects drug availability, and that variability in microbiomes between individuals could be a mechanism contributing to the variability observed between levodopa dose level and dose performance, both between and within individual patients.

Microbial metabolism of levodopa may also drive a reduction of levodopa beyond the gut. The metabolites meta-tyramine, meta-hydroxyphenylpropionic acid, and meta-hydroxyphenylacetic acid were absent from the urine of germ free rats fed levodopa, but reappeared when a microbiome was reintroduced (Goldin et al., J Pharmacol Exp Ther. 1973; 186(1):160-6) and labeled versions were generated from ¹⁴C-levodopa fed to rats (Borud et al., Acta Pharmacol Toxicol (Copenh). 1973; 33(4):308-16). A complementary study found that administration of the antibiotic neomycin to Parkinson's disease patients taking levodopa reduced the excretion of meta-hydroxyphenylacetic acid in urine (Sandler et al., Science. 1969; 166(3911):1417-8), and that administration of broad spectrum antibiotics to Parkinson's disease patients with high microbial burden or infection of the proximal gastrointestinal tract improved the response (reduction in delayed “on”/“no on”) and duration (“time on”) of levodopa therapy (Fasano et al., Mov Disord. 2013; 28(9):1241-9; Pierantozzi et al., Neurology. 2006 Jun. 27; 66(12):1824-9).

Abundance of the tdc gene in stool samples from a small cohort of patients positively correlated with both the required dose of levodopa necessary for therapeutic benefit, as well as disease duration (van Kessel et al., Nat. Commun. 2019; 10(1):310). However, using tdc gene abundance in stool as a biomarker for microbial interference has limitations, since stool may give an incomplete representation of microbial activity that occurs in the proximal small intestine (Tropini et al., Cell Host Microbe 2017; 21(4):433-442), where levodopa is absorbed. In addition, derivatives of levodopa originating from microbial metabolism have not been comprehensively identified. Metabolites derived from overlapping microbial and human metabolism of levodopa also have not been characterized in recent metabolomics studies (Branco et al., bioRxiv pre-print (posted online Apr. 23, 2018), dx.doi.org/10.1101/306266; Hertel et al., Cell Rep. 2019; 29(7):1767-1777; Hatano et al., J Neurol Neurosurg Psychiatry. 2015; 0:1-7; Luan et al., Sci Rep. 2015; 5:13888; Han et al., Mov Disord. 2017; 32(12):1720-1728).

Thus, there remains a need for biomarker-based strategies to effectively treat Parkinson's disease, particularly strategies that could stratify patients based on microbial interference with levodopa therapy. Such strategies would be useful to inform therapeutic regimens and improve treatment efficacy.

In some embodiments, the present disclosure provides methods using novel biomarker profiles to treat Parkinson's disease. In some embodiments, the present disclosure provides methods of treating Parkinson's disease in a patient in need thereof. In some embodiments, the methods disclosed herein comprise administering a levodopa therapy based on a patient's biomarker profile. In some embodiments, the levodopa therapy comprises or lacks a tyrosine decarboxylase inhibitor. In some embodiments, the biomarker profile comprises one or more biomarkers that indicate the presence and/or extent of microbial metabolism of levodopa in the patient. In some embodiments, the biomarker profile comprises meta-tyramine or a metabolic derivative thereof. In some embodiments, the methods disclosed herein comprise administering to a Parkinson's disease patient having an elevated level of microbial metabolism of levodopa, as determined from one or more biomarkers described herein (e.g., meta-tyramine or a metabolic derivative thereof), a levodopa therapy comprising a tyrosine decarboxylase inhibitor. In some embodiments, the methods disclosed herein comprise administering to a Parkinson's disease patient having a normal or low level of microbial metabolism of levodopa, as determined from one or more biomarkers described herein (e.g., meta-tyramine or a metabolic derivative thereof), a levodopa therapy lacking a tyrosine decarboxylase inhibitor. Therapeutic uses and compositions are also provided.

In some embodiments, a biomarker profile described herein comprises one or more biomarkers. In some embodiments, the biomarker profile comprises one or more metabolites derived from microbial metabolism of levodopa. In some embodiments, the one or more metabolites may be used as biomarkers to determine the presence and/or extent of microbial metabolism of levodopa in a patient (e.g., a Parkinson's disease patient). In some embodiments, the one or more metabolites may be used as biomarkers to identify the patient as suffering from microbial interference in levodopa therapy (e.g., oral levodopa therapy) and/or levodopa dose variability. In some embodiments, the one or more metabolites may be used as biomarkers to inform and provide an effective therapeutic regimen for the patient. In some embodiments, the one or more metabolites are detected and/or quantified in a biological sample (e.g., in plasma and/or urine). In some embodiments, the one or more metabolites comprise one or more circulating metabolites. In some embodiments, the one or more metabolites comprise meta-tyramine or a metabolic derivative thereof.

In some embodiments, the methods disclosed herein may be used to identify patients who may benefit from inhibition of a microbial tyrosine decarboxylase as an adjuvant therapy to levodopa treatment. In some embodiments, the methods disclosed herein inform and guide levodopa therapies, e.g., levodopa therapies comprising or lacking a tyrosine decarboxylase inhibitor. In some embodiments, the levodopa therapies described herein may allow more efficient delivery of levodopa to the central nervous system (CNS), compared to alternate therapies. In some embodiments, the levodopa therapies described herein may provide less biological variability and/or fewer side effects, compared to alternate therapies. In some embodiments, the levodopa therapies described herein may comprise a lower effective dose of levodopa, compared to alternate therapies. In some embodiments, the levodopa therapies described herein may increase efficacy and/or improve therapy performance, compared to alternate therapies. In some embodiments, the levodopa therapies described herein may reduce or eliminate the microbial metabolism of levodopa and/or increase levodopa bioavailability, compared to alternate therapies.

In some embodiments, the present disclosure provides a method of treatment, comprising administering a levodopa therapy comprising a tyrosine decarboxylase inhibitor to a Parkinson's disease patient who has an elevated level of meta-tyramine or a metabolic derivative thereof; or administering a levodopa therapy lacking a tyrosine decarboxylase inhibitor to a Parkinson's disease patient who has a normal or low level of meta-tyramine or a metabolic derivative thereof.

In some embodiments, the present disclosure provides a method of treatment, comprising administering a levodopa therapy comprising a tyrosine decarboxylase inhibitor to a Parkinson's disease patient who has an elevated level of meta-tyramine or a metabolic derivative thereof.

In some embodiments, the present disclosure provides a method of treating Parkinson's disease in a patient in need thereof, comprising: (a) determining that the patient has an elevated level of meta-tyramine or a metabolic derivative thereof; and (b) administering a levodopa therapy comprising a tyrosine decarboxylase inhibitor to the patient.

In some embodiments, the present disclosure provides a method of treating Parkinson's disease in a patient in need thereof, comprising: (a) determining that the patient has a normal or low level of meta-tyramine or a metabolic derivative thereof; and (b) administering a levodopa therapy lacking a tyrosine decarboxylase inhibitor to the patient.

In some embodiments, the present disclosure provides a method of providing a therapeutic regimen for treating Parkinson's disease in a patient in need thereof, comprising: (a) determining that the patient has an elevated level of meta-tyramine or a metabolic derivative thereof; and (b) providing a levodopa therapy comprising a tyrosine decarboxylase inhibitor to the patient.

In some embodiments, the present disclosure provides a method of providing a therapeutic regimen for treating Parkinson's disease in a patient in need thereof, comprising: (a) determining that the patient has a normal or low level of meta-tyramine or a metabolic derivative thereof; and (b) providing a levodopa therapy lacking a tyrosine decarboxylase inhibitor to the patient.

In some embodiments of the methods disclosed herein, the method (e.g., any or more of the exemplary methods described herein) further comprises obtaining a biological sample from the patient, and determining the level of meta-tyramine or a metabolic derivative thereof in the sample.

In some embodiments, the present disclosure provides a method of treating Parkinson's disease in a patient in need thereof, comprising: (a) obtaining a biological sample from the patient; (b) determining from the sample that the patient has an elevated level of meta-tyramine or a metabolic derivative thereof; and (c) administering a levodopa therapy comprising a tyrosine decarboxylase inhibitor to the patient.

In some embodiments, the present disclosure provides a method of treating Parkinson's disease in a patient in need thereof, comprising: (a) obtaining a biological sample from the patient; (b) determining from the sample that the patient has a normal or low level of meta-tyramine or a metabolic derivative thereof; and (c) administering a levodopa therapy lacking a tyrosine decarboxylase inhibitor to the patient.

In some embodiments, the present disclosure provides a method of identifying a suitable levodopa therapy for a Parkinson's disease patient, the method comprising: (a) obtaining a biological sample from the patient; (b) determining from the sample that the patient has an elevated level of meta-tyramine or a metabolic derivative thereof; and (c) identifying a levodopa therapy comprising a tyrosine decarboxylase inhibitor as a suitable levodopa therapy for the patient.

In some embodiments, the present disclosure provides a method of identifying a suitable levodopa therapy for a Parkinson's disease patient, the method comprising: (a) obtaining a biological sample from the patient; (b) determining from the sample that the patient has a normal or low level of meta-tyramine or a metabolic derivative thereof; and (c) identifying a levodopa therapy lacking a tyrosine decarboxylase inhibitor as a suitable levodopa therapy for the patient.

In some embodiments of the methods disclosed herein, the biological sample comprises a plasma sample, a urine sample, a stool sample, an intestinal sample, or a combination thereof. In some embodiments, the biological sample comprises a plasma sample, a urine sample, and/or an intestinal sample. In some embodiments, the biological sample comprises a plasma sample and a urine sample. In some embodiments, the biological sample comprises a plasma sample. In some embodiments, the plasma sample comprises peripheral blood plasma. In some embodiments, the biological sample comprises an intestinal sample. In some embodiments, the biological sample comprises an intestinal sample from the duodenum, the jejunum, the ileum, the ascending colon, the descending colon, and/or the transverse colon. In some embodiments, the intestinal sample is from the lower intestine (e.g., the ascending colon, the descending colon, and/or the transverse colon).

In some embodiments of the methods disclosed herein, the patient is receiving a levodopa therapy lacking a tyrosine decarboxylase inhibitor. In some embodiments, the level of meta-tyramine or a metabolic derivative thereof is determined less than about 5 hours after the patient is administered a single dose of the levodopa therapy lacking a tyrosine decarboxylase inhibitor. In some embodiments, the level of meta-tyramine or a metabolic derivative thereof is determined about 1 to about 3 hours after the patient is administered a single dose of the levodopa therapy lacking a tyrosine decarboxylase inhibitor.

In some embodiments of the methods disclosed herein, the level of meta-tyramine or a metabolic derivative thereof is measured by metabolomics. In some embodiments, the metabolomics comprises liquid chromatography-mass spectrometry (LC-MS), gas-phase chromatography-mass spectrometry (GC-MS), or tandem mass spectrometry (MS-MS). In some embodiments, the metabolomics comprises LC-MS. In some embodiments, the metabolomics comprises GC-MS. In some embodiments, the metabolomics comprises reversed-phase chromatography with positive ionization mode, reversed-phase chromatography with negative ionization mode, hydrophobic interaction liquid ion chromatography (HILIC) with positive ionization mode, hydrophobic interaction liquid ion chromatography (HILIC) with negative ionization mode, or a combination thereof. In some embodiments, the metabolomics comprises a combination of reversed-phase chromatography with positive ionization mode, reversed-phase chromatography with negative ionization mode, HILIC with positive ionization mode, and HILIC with negative ionization mode. In some embodiments, the level of meta-tyramine or a metabolic derivative thereof is measured by enzyme-linked immunosorbent assay (ELISA), antibody linkage, one or more other immunochemical techniques, or combinations thereof. Further, the level of meta-tyramine or a metabolic derivative thereof can be measured indirectly, for example, by using an assay that measures the level of one or more compounds, wherein the level of the one or more compounds correlates with the level of meta-tyramine or the metabolic derivative thereof.

In some embodiments of the methods disclosed herein, an elevated level of meta-tyramine or a metabolic derivative thereof in the patient is a level exceeding the level in a healthy subject naïve to levodopa; and a normal or low level of meta-tyramine or a metabolic derivative thereof in the patient is a level equal to or below the level in a healthy subject naïve to levodopa. In some embodiments, an elevated level of meta-tyramine or a metabolic derivative thereof in the patient is a level exceeding 100 ng/mL; and a normal or low level of meta-tyramine or a metabolic derivative thereof in the patient is a level equal to or below 100 ng/mL.

In some embodiments of the methods disclosed herein, the levodopa is administered simultaneously with the tyrosine decarboxylase inhibitor. In some embodiments, the levodopa is administered sequentially with the tyrosine decarboxylase inhibitor. In some embodiments, the levodopa therapy comprising a tyrosine decarboxylase inhibitor results in an increased level of circulating levodopa compared to the level of circulating levodopa prior to treatment. In some embodiments, the amount of levodopa administered in combination with the tyrosine decarboxylase inhibitor is reduced compared to the amount of levodopa administered in the absence of the tyrosine decarboxylase inhibitor. In some embodiments, the amount of levodopa administered in combination with the tyrosine decarboxylase inhibitor is reduced by at least 10% compared to the amount of levodopa administered in the absence of the tyrosine decarboxylase inhibitor. In some embodiments, the levodopa administered in combination with the tyrosine decarboxylase inhibitor is administered less frequently compared to the levodopa administered in the absence of the tyrosine decarboxylase inhibitor. In some embodiments, the levodopa administered in combination with the tyrosine decarboxylase inhibitor is administered at least 10% less frequently compared to the levodopa administered in the absence of the tyrosine decarboxylase inhibitor. In some embodiments, the treatment with levodopa in combination with the tyrosine decarboxylase inhibitor results in reduced systemic toxicity and/or improved tolerance compared to the treatment with levodopa in the absence of the tyrosine decarboxylase inhibitor.

In some embodiments of the methods disclosed herein, the levodopa therapy further comprises a peripheral aromatic amino acid decarboxylase inhibitor. In some embodiments, the peripheral aromatic amino acid decarboxylase inhibitor is carbidopa.

In some embodiments of the methods disclosed herein, the tyrosine decarboxylase inhibitor is alpha-fluoromethyltyrosine (AFMT).

In some embodiments, the tyrosine decarboxylase inhibitor is a compound chosen from the following compounds:

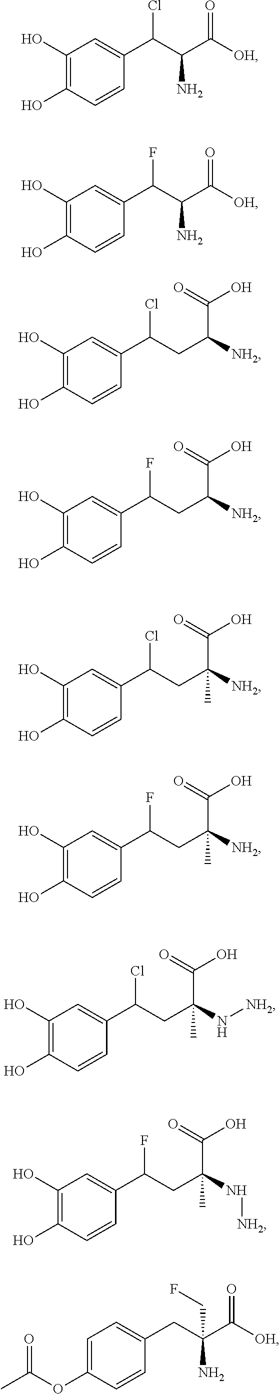

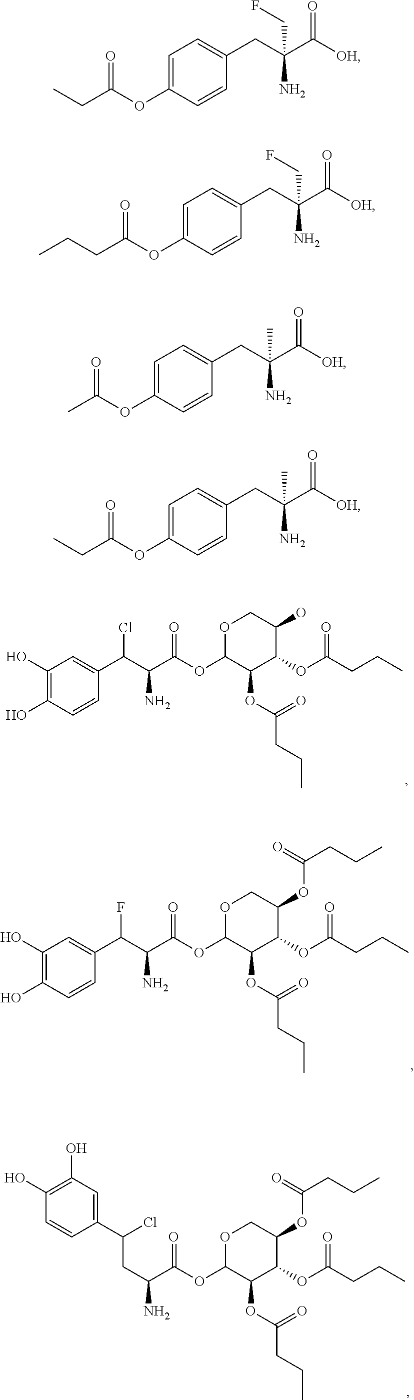

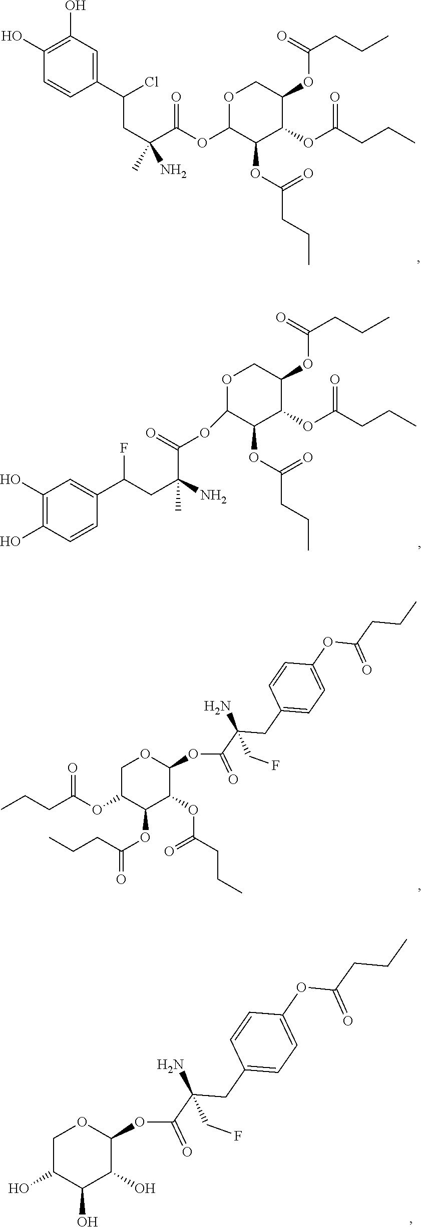

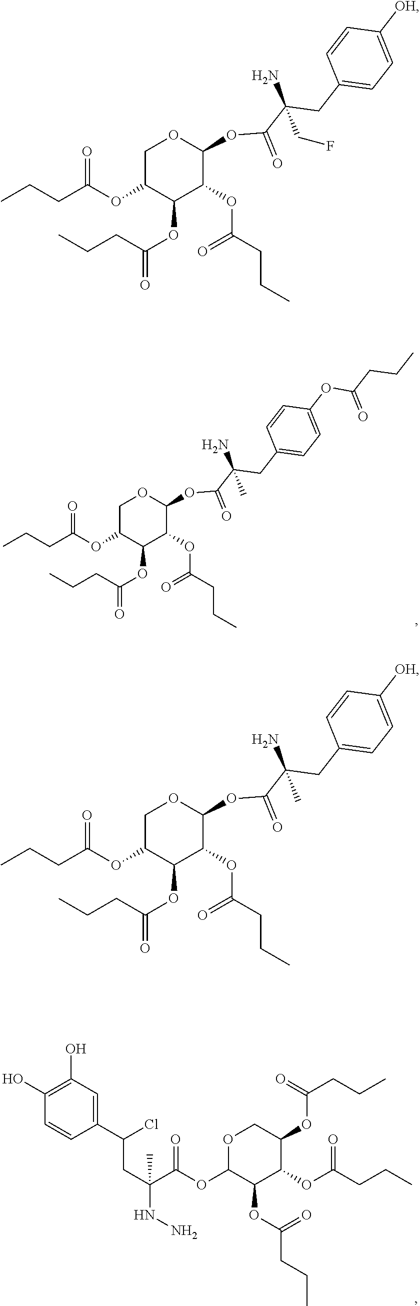

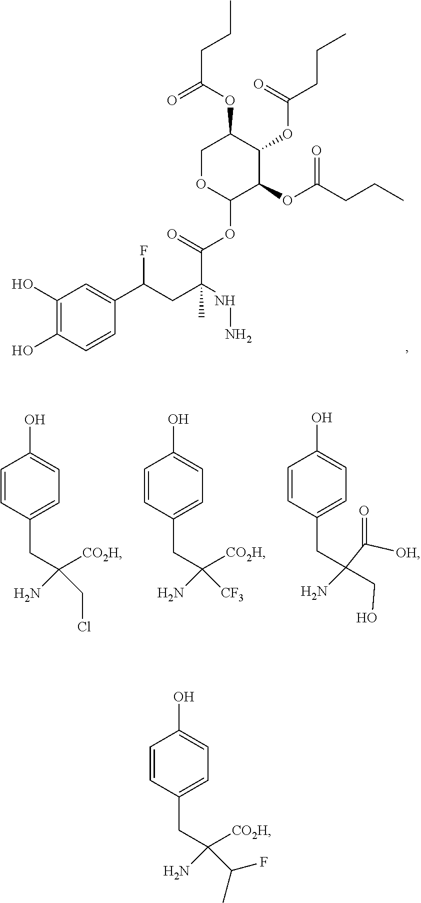 [SMILES: CCCC(=O)O[C@H]1[C@H](OC(=O)CCC)COC(OC(=O)[C@](C)(CC(F)c2ccc(O)c(O)c2)NN)[C@@H]1OC(=O)CCC ; NC(CCl)(Cc1ccc(O)cc1)C(=O)O ; NC(Cc1ccc(O)cc1)(C(=O)O)C(F)(F)F ; NC(CO)(Cc1ccc(O)cc1)C(=O)O ; CC(F)C(N)(Cc1ccc(O)cc1)C(=O)O]

and pharmaceutically acceptable salts thereof.

In some embodiments, the tyrosine decarboxylase inhibitor is a compound chosen from the following compounds:

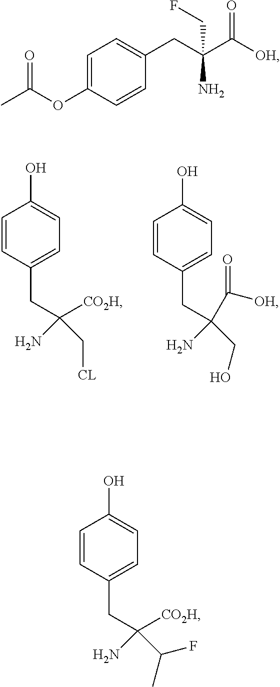

and pharmaceutically acceptable salts thereof.

In some embodiments, the tyrosine decarboxylase inhibitor is a compound chosen from the following compounds:

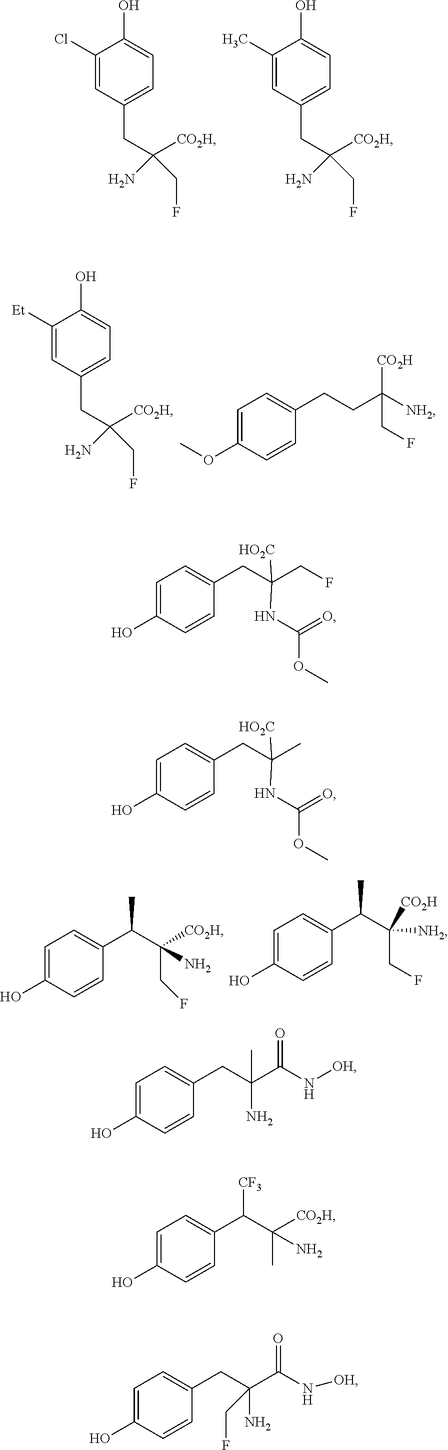

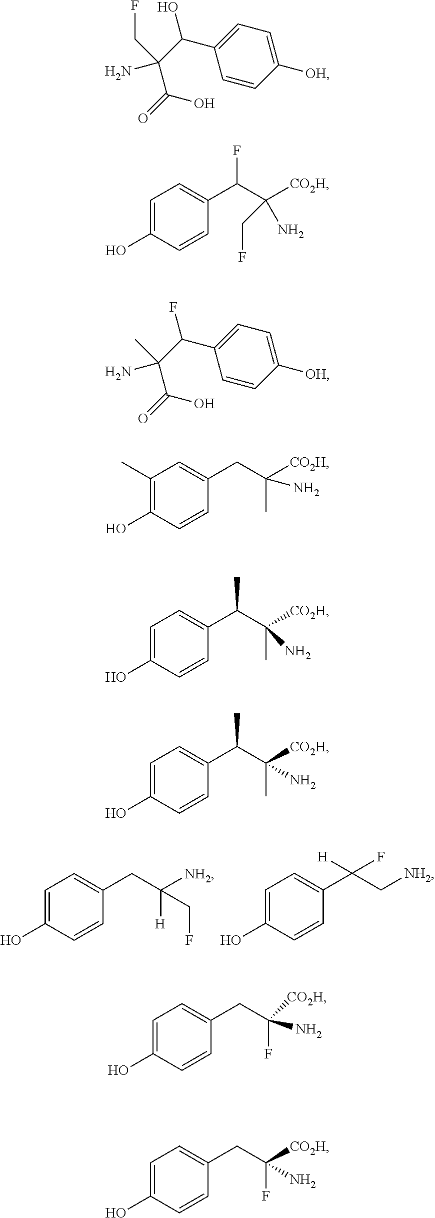

and pharmaceutically acceptable salts thereof.

In some embodiments, the tyrosine decarboxylase inhibitor is a compound chosen from the following compounds:

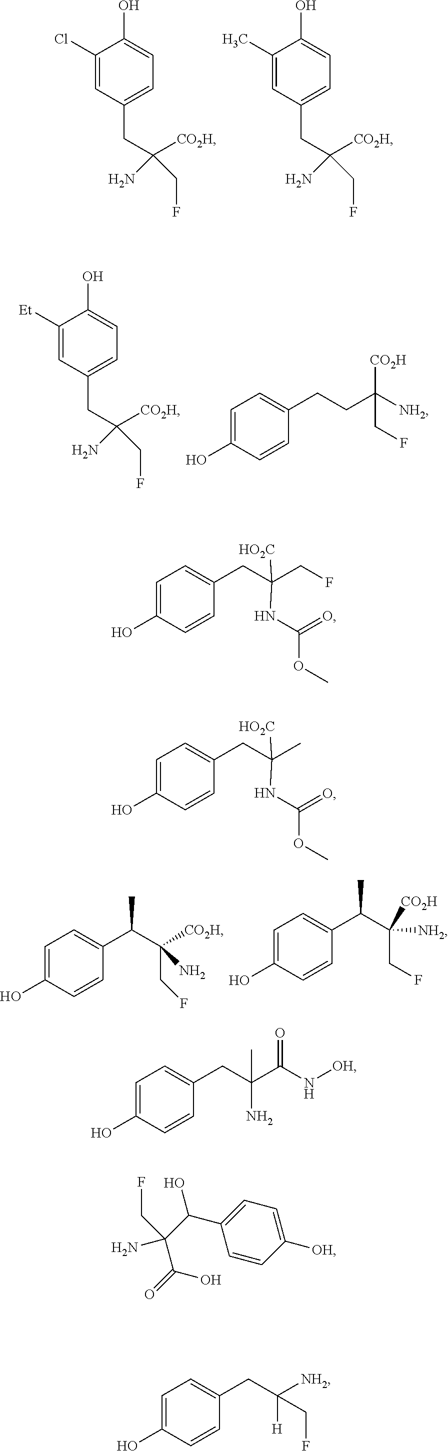

and pharmaceutically acceptable salts thereof.

In some embodiments, the tyrosine decarboxylase inhibitor is a compound of formula (I):

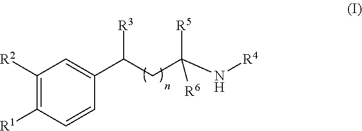

or a pharmaceutically acceptable salt thereof,
wherein

n is 0 or 1;

R¹ is H or —OR^{A}, wherein R^{A} is H, —C(O)C₁₋₆ alkyl, or an acylated sugar;

R² is H, halogen, amino, C₁₋₆ alkyl, or —OR^{A}, wherein R^{A} is H or an acylated sugar;

R³ is H, a halogen, —OH, or C₁₋₆ alkyl optionally substituted with one or more halogens;

R⁴ is H, —NH₂, —C(O)OCH₃, or an acylated sugar;

R⁵ is H, —C(O)OH, —C(O)OC₁₋₆ alkyl, —C(O)Oglycoside, —C(O)NHOH, or —C(O)O(acylated sugar); and

R⁶ is H, halogen, or optionally substituted C₁₋₆ alkyl;

provided that at least one R^{A} is present; or provided that R³ and/or R⁶ comprise a halogen.

In some embodiments, the tyrosine decarboxylase inhibitor is a compound of formula (I-a):

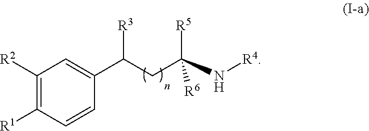

In some embodiments of formula (I) or (I-a),

n is 0 or 1;

R¹ is H, —C(O)C₁₋₆alkyl, or —OR^{A}, wherein R^{A} is H or an acylated sugar;

R² is H, or —OR^{A}, wherein R^{A} is H or an acylated sugar;

R³ is H, or a halogen;

R⁴ is H, —NH₂, or an acylated sugar;

R⁵ is —C(O)OH, —C(O)OC₁₋₆ alkyl, —C(O)Oglycoside, or —C(O)O(acylated sugar); and

R⁶ is H or optionally substituted C₁₋₆ alkyl;

provided that at least one R^{A} is present; or provided that R³ and/or R⁶ comprise a halogen.

In some embodiments of formula (I) or (I-a), R¹ is —OR^{A}. In some embodiments, R² is H or —OR^{A}. In some embodiments, each R^{A} is H. In some embodiments, R² is a halogen. In some embodiments, R³ is fluoro or chloro. In some embodiments, R³ is H. In some embodiments, R⁴ is H. In some embodiments, R⁴ is —NH₂. In some embodiments, R⁵ is —C(O)OH. In some embodiments, R⁵ is —C(O)Oacylated sugar. In some embodiments, R⁵ is H. In some embodiments, R⁶ is H. In some embodiments, R⁶ is a C₁₋₆ alkyl. In some embodiments, R⁶ is a C₁₋₆ alkyl substituted with one, two, or three halogens. In some embodiments, R⁶ is a C₁₋₆ alkyl substituted with one, two, or three fluorine atoms. In some embodiments, n is 0. In some embodiments, n is 1.

In some embodiments of formula (I) or (I-a),

n is 0;

R¹ is —OH;

R² is halogen;

R³ is H, a halogen, or —OH, C₁₋₆ alkyl optionally substituted with one or more halogens;

R⁴ is H, —NH₂, or an acylated sugar;

R⁵ is H, —C(O)OH, —C(O)OC₁₋₆ alkyl, —C(O)Oglycoside, —C(O)NHOH, or —C(O)O(acylated sugar); and

R⁶ is H or optionally substituted C₁₋₆ alkyl.

In some embodiments of formula (I) or (I-a),

n is 0;

R¹ is —OH;

R² is halogen;

R³ is H;

R⁴ is H;

R⁵ is —C(O)OH; and

R⁶ is optionally substituted alkyl.

In some embodiments of the methods disclosed herein, the meta-tyramine or a metabolic derivative thereof comprises meta-tyramine and/or at least one metabolic derivative thereof. In some embodiments, the meta-tyramine or a metabolic derivative thereof comprises meta-tyramine, 3-hydroxyphenylacetic acid, 3-hydroxyphenylacetaldehyde, 3-hydroxyphenylacetate methyl ester, 3-sulfooxyphenylacetic acid, 3-methoxyphenylacetic acid, 3-methoxyphenethylamine, 3-hydroxyphenylethanol, 3-hydroxymandelic acid, meta-octopamine, meta-tyramine-O-sulfate, and/or meta-tyramine-O-glucuronide. In some embodiments, the meta-tyramine or a metabolic derivative thereof comprises meta-tyramine, 3-hydroxyphenylacetic acid, 3-hydroxyphenylacetate methyl ester, 3-sulfooxyphenylacetic acid, 3-methoxyphenylacetic acid, 3-methoxyphenethylamine, and/or meta-tyramine-O-sulfate. In some embodiments, the meta-tyramine or a metabolic derivative thereof comprises 3-hydroxyphenylacetic acid, 3-hydroxyphenylacetate methyl ester, 3-sulfooxyphenylacetic acid, and/or meta-tyramine-O-sulfate.

### BRIEF DESCRIPTION OF THE DRAWINGS

FIG. 1A-B show the concentration and exposure of orally delivered levodopa following inhibition of microbial tyrosine decarboxylase in the rat microbiome. FIG. 1A shows pharmacokinetic curves of levodopa in circulation. FIG. 1B shows the area under the curve quantification (0-3 hours).

FIG. 2A-D show a potential pathway of microbe-initiated metabolism of levodopa in the gastrointestinal tract. FIG. 2A shows biotransformations of microbially-produced meta-tyramine. FIG. 2B shows a quantitative detection indicating enrichment of candidate biomarker compounds in a Parkinson's disease cohort. FIG. 2C shows a validation of compound identity through comparison to characterized and validated samples of known compounds (i.e., authentic standards). FIG. 2D identifies compounds produced from a hepatocyte and meta-tyramine incubation assay.

FIG. 3 shows a principal component analysis plot of signals from candidate biomarkers that discriminate between individual samples in healthy control (HC) and Parkinson's disease (PD) cohorts.

FIG. 4A-C show baseline resolutions of various compounds using metabolomics. FIG. 4A shows meta- vs. para-tyramine. FIG. 4B shows meta- vs. para-tyramine-O-Sulfate. FIG. 4C shows meta- vs. para-hydroxyphenylacetic acid.

FIG. 5 shows an exemplary validation process for Parkinson's disease plasma biomarkers, including observing hepatocyte-mediated production, matching retention time and exact mass with authentic standards, and determining the expected MS/MS fragmentation pattern.

FIG. 6 shows candidate biomarkers of microbial metabolism of levodopa detected using untargeted metabolomics. Features specific to the Parkinson's disease group (boxed) will be evaluated as additional potential biomarkers of microbial metabolism of levodopa.

FIG. 7 shows relative signals for meta-tyramine in different regions of the gastrointestinal tract in Parkinson's disease patients on levodopa therapy (PD donors) and healthy controls (HC donors). Intestinal samples were from the duodenum (Duo), jejunum (Jej), ileum (Ile), ascending colon (AC), transverse colon (TC), and descending colon (DC) in 13 HC donors (59 HC samples total) and 10 PD donors (68 PD samples total).

FIG. 8 shows heat maps for meta-tyramine signals in intestinal samples from 10 PD donors.

### DETAILED DESCRIPTION OF ILLUSTRATIVE EMBODIMENTS

The following detailed description and examples illustrate certain embodiments of the present disclosure. Those of skill in the art will recognize that there are numerous variations and modifications of this disclosure that are encompassed by its scope. Accordingly, the description of certain embodiments should not be deemed to limit the scope of the present disclosure.

In order that the disclosure may be more readily understood, certain terms are defined throughout the detailed description. Unless defined otherwise herein, all scientific and technical terms used in connection with the present disclosure have the same meaning as commonly understood by those of ordinary skill in the art.

All references cited herein, including, but not limited to, published and unpublished patent applications, granted patents, and literature references, are incorporated herein by reference and are hereby made a part of this specification. To the extent a cited reference conflicts with the disclosure herein, the specification shall control.

As used herein, the singular forms of a word also include the plural form, unless the context clearly dictates otherwise; as examples, the terms “a,” “an,” and “the” are understood to be singular or plural. By way of example, “an element” means one or more element. The term “or” shall mean “and/or” unless the specific context indicates otherwise.

The present disclosure provides, in some embodiments, methods of treating Parkinson's disease in a patient in need thereof. In some embodiments, the methods disclosed herein comprise administering a levodopa therapy based on a patient's biomarker profile. In some embodiments, the levodopa therapy comprises or lacks a tyrosine decarboxylase inhibitor. In some embodiments, the biomarker profile comprises one or more biomarkers that indicate the presence and/or extent of microbial metabolism of levodopa in the patient. In some embodiments, the biomarker profile comprises meta-tyramine or a metabolic derivative thereof. In some embodiments, the methods disclosed herein comprise administering to a Parkinson's disease patient having an elevated level of microbial metabolism of levodopa, as determined from one or more biomarkers described herein (e.g., meta-tyramine or a metabolic derivative thereof), a levodopa therapy comprising a tyrosine decarboxylase inhibitor. In some embodiments, the methods disclosed herein comprise administering to a Parkinson's disease patient having a normal or low level of microbial metabolism of levodopa, as determined from one or more biomarkers described herein (e.g., meta-tyramine or a metabolic derivative thereof), a levodopa therapy lacking a tyrosine decarboxylase inhibitor. Therapeutic uses and compositions are also provided.

In some embodiments, the biomarker profile comprises one or more biomarkers. In some embodiments, the biomarker profile comprises one or more metabolites (e.g., circulating metabolites) derived from microbial metabolism of levodopa. In some embodiments, the biomarker profile comprises one or more circulating metabolites derived from microbial metabolism of levodopa. In some embodiments, the biomarker profile comprises meta-tyramine or a metabolic derivative thereof.

In some embodiments, one or more biomarkers (e.g., meta-tyramine or a metabolic derivative thereof) are detected and/or quantified in a biological sample from a Parkinson's disease patient. In some embodiments, the one or more biomarkers comprise meta-tyramine or a metabolic derivative thereof. In some embodiments, the presence and/or level of meta-tyramine or a metabolic derivative thereof in a biological sample from a Parkinson's disease patient (e.g., in a plasma sample, a urine sample, or both) indicates the presence and/or extent of microbial metabolism of levodopa in the patient. In some embodiments, this metabolic activity may affect the efficacy of a levodopa therapy that the patient is already receiving or may receive. In some embodiments, the level of meta-tyramine or a metabolic derivative thereof is determined less than about 5 hours (e.g., about 1 to about 3 hours) after the patient is administered a single dose of a levodopa therapy. In some embodiments, the level of meta-tyramine or a metabolic derivative thereof in a biological sample from the patient is compared to the level of meta-tyramine or a metabolic derivative thereof in a reference sample. In some embodiments, the reference sample is from a healthy subject naïve to levodopa. In some embodiments, the reference sample is from a Parkinson's disease patient naïve to or not currently on a levodopa therapy. In some embodiments, this comparison may be used to determine the origin of the metabolites and/or confirm that the metabolites result from microbial metabolism of levodopa.

In some embodiments, the biomarkers described herein may enable identification of Parkinson's disease patients that would benefit from inhibition of the microbiome's ability to metabolize levodopa. In some embodiments, the biomarkers described herein may be used to assess the microbiome's impact on one or more clinical parameters of a levodopa therapy. In some embodiments, the strength of the relationship between the biomarkers described herein and corresponding patient metadata (e.g., levodopa dose amount, dose frequency, length of therapy use, antibiotic history, overall efficacy of therapy (e.g., On-Off times, dose failures, etc.), and/or differences in MDS-UPDRS On-Off score) may be evaluated and/or quantified. In some embodiments, this analysis may help elucidate the relationship between microbial activity in the gastrointestinal tract (e.g., in the small intestine) and efficacy of a levodopa therapeutic regimen.

In some embodiments, the biomarkers described herein may be used to stratify patients based on the presence and/or extent of microbial metabolism of levodopa. In some embodiments, the biomarkers described herein may be used to identify patients suffering from microbial interference in levodopa therapy (e.g., oral levodopa therapy) and/or levodopa dose variability. In some embodiments, the biomarkers described herein may be used to inform and provide an effective therapeutic regimen for Parkinson's disease patients. In some embodiments, the biomarkers described herein allow more efficient delivery of levodopa to the central nervous system, with less biological variability and/or fewer side effects.

The term “biomarker,” as used herein, refers to a biological compound that is present in a biological sample and may be isolated from, or measured in, the biological sample. In some embodiments, a biomarker is an amino acid or an amino acid derivative, e.g., meta-tyramine, or a metabolic derivative thereof. Other exemplary biomarker types include, but are not limited to, small molecules, nucleic acids, polynucleotides, peptides, polypeptides, proteins, proteoglycans, glycoproteins, lipoproteins, carbohydrates, lipids, organic or inorganic chemicals, and natural polymers. A biomarker is considered to be informative if a measurable aspect of the biomarker is associated with a given state of a patient (e.g., a Parkinson's disease patient), such as the presence and/or extent of microbial metabolism of levodopa. Exemplary measurable aspects may include, for example, the presence, absence, or level of the biomarker in a biological sample from the patient and/or its presence as part of a profile of biomarkers. Such measurable aspects of a biomarker may be referred to herein as “features.” A feature may also be a ratio of two or more measurable aspects of biomarkers, for example. A “biomarker profile” comprises at least two features, wherein the features can correspond to the same type of biomarker (e.g., two amino acids) or different types of biomarkers (e.g., an amino acid and a polynucleotide). In some embodiments, a biomarker profile may comprise features of two or more metabolites that result from microbial metabolism of levodopa. A biomarker profile, in some embodiments, may also comprise at least 5, 10, 20, 30, 40, 50 or more features. In some embodiments, a biomarker profile comprises features of meta-tyramine or a metabolic derivative thereof, alone or in combination with one or more additional features.

The profile of biomarkers obtained from a patient, i.e., the test biomarker profile, may be compared to a reference biomarker profile. A reference biomarker profile can be generated from one individual or a population or cohort of two or more individuals. The population or cohort, for example, may comprise 5, 10, 15, 18, 20, 30, 40, 50, 75, 100 or more individuals. Furthermore, the reference biomarker profile and the patient's (test) biomarker profile that are compared in the methods disclosed herein may be generated from the same individual, provided that the test and the reference biomarker profiles are generated from biological samples taken at different time points and compared to one another. For example, a sample may be obtained from a patient before the start of a treatment period. A reference biomarker profile taken from that sample may then be compared to biomarker profiles generated from subsequent samples from the same individual after receiving treatment. Such a comparison may be used, for example, to determine the status of microbial metabolism of levodopa in the individual by repeated classifications over time. In some embodiments, the reference individual or population may be a healthy subject naïve to levodopa therapy, or a population of healthy subjects naïve to levodopa therapy. In some embodiments, the reference individual or population may be a Parkinson's disease patient naïve to or not currently on a levodopa therapy, or a population of Parkinson's disease patients naïve to or not currently on a levodopa therapy.

In some embodiments, the methods disclosed herein comprise comparing a patient's biomarker profile with a reference biomarker profile. As used herein, such a “comparison” includes any means to discern at least one difference between the patient's biomarker profile and the reference biomarker profile. In some embodiments, a comparison may include a visual inspection of chromatographic spectra. In some embodiments, a comparison may include arithmetical or statistical comparisons of values assigned to features of the profiles. For instance, in some embodiments, a comparison may include arithmetical or statistical comparisons of levels (e.g., concentrations) of particular metabolites. In some embodiments, the comparison can indicate the presence and/or extent of microbial metabolism of levodopa in the patient. In some embodiments, the comparison can help determine a suitable levodopa therapy for the patient and/or predict the patient's responsiveness to treatment with a particular levodopa therapy (e.g., a levodopa therapy comprising or lacking a tyrosine decarboxylase inhibitor). In some embodiments, the comparison can inform and help determine an effective therapeutic regimen for the patient.

The term “authentic standard,” as used herein, refers to a characterized and validated sample of a known compound. For example, in some embodiments, to show that 3-hydroxyphenylacetic acid is present in a plasma sample, the chromatogram of the plasma sample may be compared to and matched with the chromatogram of a purified sample of 3-hydroxyphenylacetic acid. In such embodiments, the purified sample of 3-hydroxyphenylacetic acid is the authentic standard.

The term “levodopa,” also known as “L-DOPA,” refers to L-3,4-dihydroxyphenylalanine, which is an amino acid precursor in the biosynthetic pathway of dopamine, norepinephrine (noradrenaline), and epinephrine (adrenaline) (collectively known as catecholamines). The structure of levodopa is as follows:

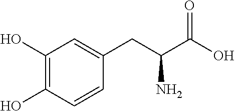 [SMILES: N[C@@H](Cc1ccc(O)c(O)c1)C(=O)O]

The term “levodopa therapy,” as used herein, refers to any therapeutic regimen comprising administration of levodopa. In some embodiments, levodopa is administered alone. In some embodiments, levodopa is administered in combination with one or more additional therapeutic agents (e.g., a tyrosine decarboxylase inhibitor, a peripheral aromatic amino acid decarboxylase inhibitor, or both). Exemplary therapeutic agents suitable for use with levodopa are described herein and others are known in the art.

The term “levodopa therapy comprising a tyrosine decarboxylase inhibitor,” as used herein, refers to any therapeutic regimen comprising administration of levodopa in combination with a tyrosine decarboxylase inhibitor. In some embodiments, levodopa and a tyrosine decarboxylase inhibitor are administered in combination with one or more additional therapeutic agents. For instance, in some embodiments, levodopa and a tyrosine decarboxylase inhibitor are administered in combination with a peripheral aromatic amino acid decarboxylase inhibitor. In some embodiments, the peripheral aromatic amino acid decarboxylase inhibitor is carbidopa.

The term “levodopa therapy lacking a tyrosine decarboxylase inhibitor,” as used herein, refers to any therapeutic regimen comprising administration of levodopa without a tyrosine decarboxylase inhibitor. In some embodiments, levodopa is administered alone. In some embodiments, levodopa is administered in combination with one or more alternative additional therapeutic agents (i.e., additional therapeutic agents that do not comprise a tyrosine decarboxylase inhibitor). In some embodiments, levodopa is administered in combination with a peripheral aromatic amino acid decarboxylase inhibitor. In some embodiments, the peripheral aromatic amino acid decarboxylase inhibitor is carbidopa.

The terms “Parkinson's disease” and “PD,” as used herein, refer to a progressive, neurodegenerative disorder that affects the mobility and control of the skeletal muscular system. Clinically, Parkinson's disease is typically characterized by severe and progressing tremors, rigidity, bradykinetic movements, posture instability, and cognitive impairment. Neuropathologically, the hallmarks of Parkinson's disease can include the progressive degeneration of dopaminergic nigrostriatal neurons and the formation of aggregated α-synuclein, called Lewy bodies, in the brain. Treatments, such as levodopa therapies, may improve one or more symptoms of Parkinson's disease in a patient.

The terms “patient” and “subject” are used interchangeably herein to refer to a human or non-human animal (e.g., a mammal). As used herein, the term “Parkinson's disease patient” refers to a patient that is suffering from or is at risk of developing Parkinson's disease, as determined by a qualified professional (e.g., a doctor or a nurse practitioner).

The term “peripheral aromatic amino acid decarboxylase inhibitor,” as used herein, refers to any compound capable of reducing or inhibiting aromatic amino acid decarboxylation in the peripheral nervous system. In some embodiments, conversion of levodopa into dopamine is catalyzed by an aromatic amino acid decarboxylase enzyme. In some embodiments, the conversion can be blocked by a peripheral aromatic amino acid decarboxylase inhibitor. In some embodiments, a peripheral aromatic amino acid decarboxylase inhibitor reduces or eliminates the activity of an aromatic amino acid decarboxylase enzyme. In some embodiments, a peripheral aromatic amino acid decarboxylase inhibitor reduces the activity of an aromatic amino acid decarboxylase enzyme by at least about 10%, 20%, 30%, 40%, 50%, 60%, 70%, 80%, 90%, 95%, 99%, or 100%, relative to the activity of the enzyme in the absence of the peripheral aromatic amino acid decarboxylase inhibitor. Exemplary peripheral aromatic amino acid decarboxylase inhibitors include benserazide and carbidopa. In some embodiments, the peripheral aromatic amino acid decarboxylase inhibitor comprises carbidopa. In some embodiments, carbidopa inhibits decarboxylation of peripheral levodopa. Carbidopa may be designated chemically as (−)-L-α-hydrazino-α-methyl-β-(3,4-dihydroxybenzene) propanoic acid monohydrate. The empirical formula of carbidopa is C₁₀H₁₄N₂O₄H₂O and the structure of carbidopa is as follows:

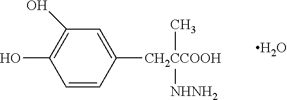

The terms “treat,” “treatment,” and “treating,” as used herein, refer to the medical management of a subject with the intent to improve, ameliorate, stabilize, or cure a disease, disorder, or condition (e.g., Parkinson's disease). These terms include active treatment (treatment directed to improve the disease, disorder, or condition); causal treatment (treatment directed to the cause of the associated disease, disorder, or condition); palliative treatment (treatment designed for the relief of symptoms of the disease, disorder, or condition); preventative treatment (treatment directed to minimizing or partially or completely inhibiting the development of the associated disease, disorder, or condition); and supportive treatment (treatment employed to supplement another therapy). An exemplary disease, disorder, or condition is Parkinson's disease.

The term “tyrosine decarboxylase inhibitor,” as used herein, refers to any compound capable of reducing or inhibiting the conversion of levodopa to dopamine by a tyrosine decarboxylase enzyme. In some embodiments, a tyrosine decarboxylase inhibitor reduces or eliminates the activity of a tyrosine decarboxylase enzyme. In some embodiments, a tyrosine decarboxylase inhibitor reduces the activity of a tyrosine decarboxylase enzyme by at least about 10%, 20%, 30%, 40%, 50%, 60%, 70%, 80%, 90%, 95%, 99%, or 100%, relative to the activity of the enzyme in the absence of the tyrosine decarboxylase inhibitor. In some embodiments, the tyrosine decarboxylase enzyme is a tyrosine decarboxylase from Enterococcus faecalis. In some embodiments, the tyrosine decarboxylase inhibitor is alpha-fluoromethyltyrosine (AFMT). In some embodiments, the tyrosine decarboxylase inhibitor is any of the exemplary compounds shown and described in PCT/US2019/064896, which is incorporated herein by reference for all its disclosed compounds and methods of synthesizing those compounds. In some embodiments, the tyrosine decarboxylase inhibitor is any of the exemplary compounds described or incorporated by reference herein.

Generation of Biomarker Profiles

In some embodiments, the methods disclosed herein comprise obtaining a profile of biomarkers from a biological sample taken from a patient (e.g., a Parkinson's disease patient). A biological sample may be blood, plasma, saliva, serum, sputum, urine, cerebral spinal fluid, cells, a cellular extract, a tissue sample, a tissue biopsy, a stool sample, or a combination thereof. In some embodiments, the biological sample comprises a plasma sample, a urine sample, a stool sample, an intestinal sample, or a combination thereof. In some embodiments, the biological sample comprises a plasma sample, a urine sample, and/or an intestinal sample. In some embodiments, the biological sample comprises a plasma sample and a urine sample. In some embodiments, the biological sample comprises a plasma sample. In some embodiments, the plasma sample comprises peripheral blood plasma (i.e., plasma from peripheral blood, i.e., blood that circulates throughout the body). In some embodiments, the biological sample comprises an intestinal sample (i.e., a sample from one or more regions of a gastrointestinal tract, e.g., the human gastrointestinal tract, e.g., the duodenum, the jejunum, the ileum, the ascending colon, the descending colon, and/or the transverse colon). In some embodiments, the biological sample comprises an intestinal sample from the duodenum, the jejunum, the ileum, the ascending colon, the descending colon, and/or the transverse colon. In some embodiments, the intestinal sample is from the lower intestine (e.g., the ascending colon, the descending colon, and/or the transverse colon). In some embodiments, the biological sample comprises a stool sample. A reference biomarker profile may also be obtained or used, for example, from an individual or a population of individuals. In some embodiments, the reference biomarker profile is obtained or used from a healthy subject naïve to levodopa therapy, or a population of healthy subjects naïve to levodopa therapy. In some embodiments, a reference biomarker profile is obtained or used from a Parkinson's disease patient naïve to or not currently on a levodopa therapy. In some embodiments, a reference biomarker profile is obtained or used from a population of Parkinson's disease patients naïve to or not currently on a levodopa therapy. In some embodiments, a reference biomarker profile is obtained or used from a Parkinson's disease patient that was recently on or is currently on a levodopa therapy. In some embodiments, a reference biomarker profile is obtained or used from a population of Parkinson's disease patients recently on or currently on a levodopa therapy.

Biomarker profiles may be generated by the use of one or more separation methods. For example, suitable separation methods may include a mass spectrometry method, such as liquid chromatography-mass spectrometry (LC-MS), gas-phase chromatography-mass spectrometry (GC-MS), or tandem mass spectrometry (MS-MS). Other suitable separation methods may include reversed-phase chromatography (e.g., with positive and/or negative ionization mode) and hydrophobic interaction liquid ion chromatography (HILIC) (e.g., with positive and/or negative ionization mode), or a combination thereof. In some embodiments, the biological sample may be fractionated prior to application of the separation method. Biomarker profiles may also be generated by methods that do not require physical separation of the biomarkers themselves. For example, nuclear magnetic resonance (NMR) spectroscopy may be used to resolve a profile of biomarkers from a complex mixture of molecules.

Biomarkers

Biomarkers that can be used in the methods of the present disclosure include those indicative of the presence and/or extent of microbial metabolism of levodopa. Exemplary methods for identifying valid and applicable biomarkers (also referred to “biomarker quantification”) are described herein. Exemplary methods and considerations for biomarker quantification are also reviewed in Koulman et al. (Anal Bioanal Chem. 2009; 394(3):663-670).

In some embodiments, a biomarker or biomarker profile described herein comprises low molecular weight compounds, such as metabolites. In some embodiments, a biomarker or biomarker profile described herein comprises metabolites of levodopa. In some embodiments, a biomarker or biomarker profile described herein comprises microbial-specific metabolites of levodopa.

In some embodiments, a biomarker or biomarker profile described herein comprises meta-tyramine or a metabolic derivative thereof.

In some embodiments, meta-tyramine or a metabolic derivative thereof comprises meta-tyramine, 3-hydroxyphenylacetic acid, 3-hydroxyphenylacetaldehyde, 3-hydroxyphenylacetate methyl ester, 3-sulfooxyphenylacetic acid, 3-methoxyphenylacetic acid, 3-methoxyphenethylamine, 3-hydroxyphenylethanol, 3-hydroxymandelic acid, meta-octopamine, meta-tyramine-O-sulfate, and/or meta-tyramine-O-glucuronide. In some embodiments, meta-tyramine or a metabolic derivative thereof comprises meta-tyramine, 3-hydroxyphenylacetic acid, 3-hydroxyphenylacetate methyl ester, 3-sulfooxyphenylacetic acid, 3-methoxyphenylacetic acid, 3-methoxyphenethylamine, and/or meta-tyramine-O-sulfate. In some embodiments, meta-tyramine or a metabolic derivative thereof comprises 3-hydroxyphenylacetic acid, 3-hydroxyphenylacetate methyl ester, 3-sulfooxyphenylacetic acid, and/or meta-tyramine-O-sulfate.

Useful biomarkers may also include those that have not yet been identified or associated with a relevant physiological state. In some embodiments, useful biomarkers are identified as components of a biomarker profile from a biological sample, e.g., using any of the exemplary biomarker identification/quantification methods described herein. In some embodiments, one or more features of a candidate biomarker can be further characterized, e.g., to determine the molecular structure of the biomarker. Methods for such structural characterization are well-known in the art and include, for example, high-resolution mass spectrometry, infrared spectrometry, ultraviolet spectrometry, and nuclear magnetic resonance.

In some embodiments, the methods disclosed herein comprise detecting a biomarker or biomarker profile in a biological sample taken from a patient. In some embodiments, the methods disclosed herein comprise acquiring targeted features (e.g., compounds based on a curated standard library) and untargeted features (e.g., compounds of unknown identity) that are detected in the biological sample. In some embodiments, this approach allows the measurement of not only a priori biomarkers such as meta-tyramine and metabolic derivatives thereof, but also any differentially abundant features between sample and/or sample cohorts not initially anticipated. In some embodiments, one or more differentially abundant compounds are identified and/or investigated to verify origin from levodopa and the microbiome. In some embodiments, one or more differentially abundant compounds are identified by performing MS/MS analysis on one or more unknown peaks. In some embodiments, the identified compounds are synthesized, validated, and/or quantified. In some embodiments, healthy control samples are used to exclude compounds derived from non-levodopa sources.

In some embodiments, a list of one or more compounds determined to originate from microbial metabolism of levodopa is compiled. In some embodiments, one or more of the compounds are profiled across affected patients (e.g., Parkinson's disease patients) as well as healthy control subjects to confirm the compound is limited to the affected group. In some embodiments, any compounds having an ambiguous profile, such as those that may overlap with endogenous or dietary sources of levodopa, are eliminated. In some embodiments, one or more of the remaining compounds are validated as biomarkers.

In some embodiments, validation comprises confirming that a compound (i.e., a candidate biomarker) is derived exclusively from microbial metabolism of levodopa. In some embodiments, validation comprises examining the presence of the candidate biomarker in human biological samples. In some embodiments, validation comprises producing the compound from a precursor in a series of Drug Metabolism Identification (MetID) assays (e.g., human liver microsomes, liver S9 fractions, hepatocytes, kidney, or intestinal microsomes). In some embodiments, the appearance of a compound in an in vitro assay (e.g., a MetID assay in hepatocytes or microsomes) may be used to assess and/or confirm that the compound is a product of the metabolism of levodopa by the microbiome of the host.

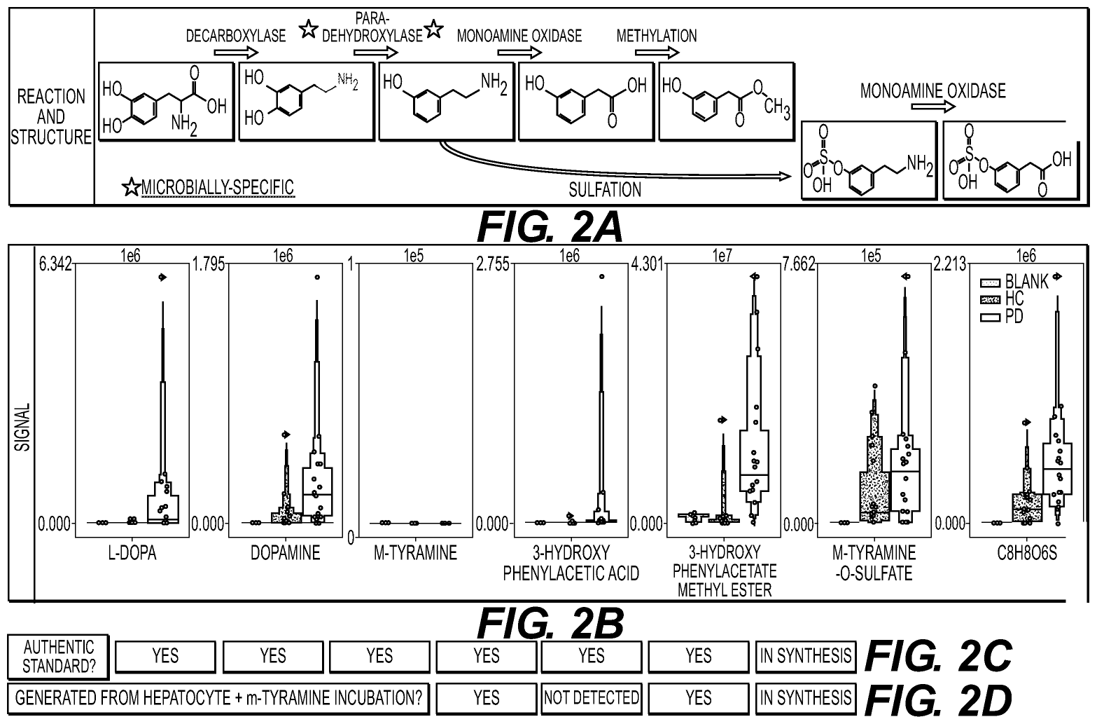

In some embodiments, validation is performed using any of the exemplary methods described herein, such as those exemplified herein using meta-tyramine and hepatocytes (see, e.g., Example 6; see also Example 8 and FIG. 2D). In some embodiments, agreement between the compound and a product in an in vitro assay (e.g., a MetID assay in hepatocytes or microsomes) incubated with the microbial precursor may establish the compound as a product of the metabolism of levodopa by the microbiome of the host. In some embodiments, if the compound is not detected in the in vitro assay, the compound may be further evaluated for its possible origin through microbial metabolism converting levodopa into a product other than meta-tyramine and/or through a second round of microbial metabolism enabled by recirculation of the compound back from the liver to the gastrointestinal system, e.g., through enterohepatic recirculation. In some embodiments, gut bacteria and metabolism are evaluated via the incubation of dominant bacterial products (e.g., meta-tyramine) in a variety of host metabolic conditions that can feed into systemic circulation. In some embodiments, this approach may be used to assess the combined bacterial-host metabolism of levodopa and/or improve the understanding of the fate of levodopa in humans.

In some embodiments, one or more compounds detected in a biological sample and validated as being derived from microbial products are identified as biomarkers of microbial metabolism of levodopa. In some embodiments, the one or more compounds have a uniquely microbial signature. In some embodiments, the one or more compounds are direct products resulting from microbial activity, from host metabolism on microbial-specific metabolites, or both. In some embodiments, the one or more compounds are detected in one or more sample types (e.g., in plasma and/or urine samples) with high specificity and/or sensitivity to affected patients (e.g., Parkinson's disease patients), e.g., compared to healthy control subjects. In some embodiments, the one or more compounds comprise meta-tyramine or a metabolic derivative thereof.

In some embodiments, quantitative values of biomarkers and a proposed metabolic map of metabolites may be used as inputs to calculate the extent of microbial metabolism of levodopa in a patient. In some embodiments, the extent of microbial metabolism is approximated by calculating the amount of levodopa metabolized relative to the amount of levodopa remaining and comparing to the known dose. In some embodiments, the results of this analysis are used to determine the prevalence and/or predominance of the metabolism of levodopa in a heterogenous population. In some embodiments, the prevalence of different biotransformation pathways is also investigated. In some embodiments, corresponding metadata associated with patients and patient samples (e.g., levodopa dose amount, dose frequency, length of therapy use, antibiotic history, overall efficacy of therapy (e.g., On-Off times, dose failures), and/or differences in MDS-UPDRS On-Off score) may be used to identify parameters predictive of therapeutic interference from the microbiome.

In some embodiments, establishing a quantitative estimate of compounds derived from microbial metabolism of levodopa comprises comprehensive acquisition of authentic standards, as well as accurate calibration of LC-MS signals in a sample matrix to estimate exact concentrations within the samples. In some embodiments, establishing a quantitative estimate comprises GC-MS. In some embodiments, the GC-MS provides higher sensitivity.

In some embodiments, a predictive model is generated based on microbially-derived metabolites of levodopa in order to assess the extent of microbial metabolism of levodopa in each patient. In some embodiments, based on this output, summary statistics of the proportion of patients that would be expected to derive a therapeutic benefit from reducing or inhibiting the microbial metabolism of levodopa may be compiled. In some embodiments, commonalities between patients based on provided metadata may also be determined.

Therapeutic Methods and Uses

In some embodiments, the methods disclosed herein are useful for screening Parkinson's disease patients expected to derive a therapeutic benefit from reducing or inhibiting the microbial metabolism of levodopa. In some embodiments, the methods disclosed herein are useful for stratifying a population of Parkinson's disease patients according to the contribution of their microbiome in metabolizing levodopa. In some embodiments, such stratification may help define a clinical population in which a tyrosine decarboxylase inhibitor or another adjuvant therapeutic capable of reducing or inhibiting the microbial metabolism of levodopa will be effective. In some embodiments, leveraging the quantitative values of the biomarkers and biomarker profiles described herein (e.g., biomarkers and biomarker profiles comprising meta-tyramine or a metabolic derivative thereof) in combination with metadata from each patient may provide a comprehensive view of the extent and/or variability of microbial metabolism of levodopa across individuals.

In some embodiments, the present disclosure provides a method of treatment, comprising administering a levodopa therapy comprising a tyrosine decarboxylase inhibitor to a Parkinson's disease patient who has an elevated level of meta-tyramine or a metabolic derivative thereof; or administering a levodopa therapy lacking a tyrosine decarboxylase inhibitor to a Parkinson's disease patient who has a normal or low level of meta-tyramine or a metabolic derivative thereof. In some embodiments, the present disclosure provides use of meta-tyramine or a metabolic derivative thereof as a biomarker for treatment. In some embodiments, the present disclosure provides use of meta-tyramine or a metabolic derivative thereof as a biomarker in the manufacture of a medicament for treatment. In some embodiments, the present disclosure provides meta-tyramine or a metabolic derivative thereof for use as a biomarker for treatment. In some embodiments, the treatment comprises administering a levodopa therapy comprising a tyrosine decarboxylase inhibitor to a Parkinson's disease patient who has an elevated level of meta-tyramine or a metabolic derivative thereof; or administering a levodopa therapy lacking a tyrosine decarboxylase inhibitor to a Parkinson's disease patient who has a normal or low level of meta-tyramine or a metabolic derivative thereof.

In some embodiments, the present disclosure provides a method of treatment, comprising administering a levodopa therapy comprising a tyrosine decarboxylase inhibitor to a Parkinson's disease patient who has an elevated level of meta-tyramine or a metabolic derivative thereof. In some embodiments, the present disclosure provides use of meta-tyramine or a metabolic derivative thereof as a biomarker for treatment. In some embodiments, the present disclosure provides use of meta-tyramine or a metabolic derivative thereof as a biomarker in the manufacture of a medicament for treatment. In some embodiments, the present disclosure provides meta-tyramine or a metabolic derivative thereof for use as a biomarker for treatment. In some embodiments, the treatment comprises administering a levodopa therapy comprising a tyrosine decarboxylase inhibitor to a Parkinson's disease patient who has an elevated level of meta-tyramine or a metabolic derivative thereof.

In some embodiments, the present disclosure provides a method of treating Parkinson's disease in a patient in need thereof, comprising: (a) determining that the patient has an elevated level of meta-tyramine or a metabolic derivative thereof; and (b) administering a levodopa therapy comprising a tyrosine decarboxylase inhibitor to the patient. In some embodiments, the present disclosure provides use of meta-tyramine or a metabolic derivative thereof as a biomarker for treating Parkinson's disease in a patient in need thereof. In some embodiments, the present disclosure provides use of meta-tyramine or a metabolic derivative thereof as a biomarker in the manufacture of a medicament for treating Parkinson's disease in a patient in need thereof. In some embodiments, the present disclosure provides meta-tyramine or a metabolic derivative thereof for use as a biomarker for treating Parkinson's disease in a patient in need thereof. In some embodiments, treating comprises: (a) determining that the patient has an elevated level of meta-tyramine or a metabolic derivative thereof; and (b) administering a levodopa therapy comprising a tyrosine decarboxylase inhibitor to the patient.

In some embodiments, the present disclosure provides a method of treating Parkinson's disease in a patient in need thereof, comprising: (a) determining that the patient has a normal or low level of meta-tyramine or a metabolic derivative thereof; and (b) administering a levodopa therapy lacking a tyrosine decarboxylase inhibitor to the patient. In some embodiments, the present disclosure provides use of meta-tyramine or a metabolic derivative thereof as a biomarker for treating Parkinson's disease in a patient in need thereof. In some embodiments, the present disclosure provides use of meta-tyramine or a metabolic derivative thereof as a biomarker in the manufacture of a medicament for treating Parkinson's disease in a patient in need thereof. In some embodiments, the present disclosure provides meta-tyramine or a metabolic derivative thereof for use as a biomarker for treating Parkinson's disease in a patient in need thereof. In some embodiments, treating comprises: (a) determining that the patient has a normal or low level of meta-tyramine or a metabolic derivative thereof; and (b) administering a levodopa therapy lacking a tyrosine decarboxylase inhibitor to the patient.

In some embodiments, the present disclosure provides a method of treatment, comprising: (a) determining that a Parkinson's disease patient has an elevated level of meta-tyramine or a metabolic derivative thereof, or determining that a Parkinson's disease patient has a normal or low level of meta-tyramine or a metabolic derivative thereof; and (b) administering a levodopa therapy comprising a tyrosine decarboxylase inhibitor to the Parkinson's disease patient who has an elevated level of meta-tyramine or a metabolic derivative thereof, or administering a levodopa therapy lacking a tyrosine decarboxylase inhibitor to the Parkinson's disease patient who has a normal or low level of meta-tyramine or a metabolic derivative thereof. In some embodiments, the present disclosure provides use of meta-tyramine or a metabolic derivative thereof as a biomarker for treatment. In some embodiments, the present disclosure provides use of meta-tyramine or a metabolic derivative thereof as a biomarker in the manufacture of a medicament for treatment. In some embodiments, the present disclosure provides meta-tyramine or a metabolic derivative thereof for use as a biomarker for treatment. In some embodiments, the treatment comprises: (a) determining that a Parkinson's disease patient has an elevated level of meta-tyramine or a metabolic derivative thereof, or determining that a Parkinson's disease patient has a normal or low level of meta-tyramine or a metabolic derivative thereof; and (b) administering a levodopa therapy comprising a tyrosine decarboxylase inhibitor to the Parkinson's disease patient who has an elevated level of meta-tyramine or a metabolic derivative thereof, or administering a levodopa therapy lacking a tyrosine decarboxylase inhibitor to the Parkinson's disease patient who has a normal or low level of meta-tyramine or a metabolic derivative thereof.

In some embodiments, the present disclosure provides a method of providing a therapeutic regimen for treating Parkinson's disease in a patient in need thereof, comprising: (a) determining that the patient has an elevated level of meta-tyramine or a metabolic derivative thereof; and (b) providing a levodopa therapy comprising a tyrosine decarboxylase inhibitor to the patient. In some embodiments, the present disclosure provides use of meta-tyramine or a metabolic derivative thereof as a biomarker for providing a therapeutic regimen. In some embodiments, the present disclosure provides use of meta-tyramine or a metabolic derivative thereof as a biomarker in the manufacture of a medicament for providing therapeutic regimen. In some embodiments, the present disclosure provides meta-tyramine or a metabolic derivative thereof for use as a biomarker for providing a therapeutic regimen. In some embodiments, providing a therapeutic regimen comprises: (a) determining that the patient has an elevated level of meta-tyramine or a metabolic derivative thereof; and (b) providing a levodopa therapy comprising a tyrosine decarboxylase inhibitor to the patient.

In some embodiments, the present disclosure provides a method of providing a therapeutic regimen for treating Parkinson's disease in a patient in need thereof, comprising: (a) determining that the patient has a normal or low level of meta-tyramine or a metabolic derivative thereof; and (b) providing a levodopa therapy lacking a tyrosine decarboxylase inhibitor to the patient. In some embodiments, the present disclosure provides use of meta-tyramine or a metabolic derivative thereof as a biomarker for providing a therapeutic regimen. In some embodiments, the present disclosure provides use of meta-tyramine or a metabolic derivative thereof as a biomarker in the manufacture of a medicament for providing a therapeutic regimen. In some embodiments, the present disclosure provides meta-tyramine or a metabolic derivative thereof for use as a biomarker for providing a therapeutic regimen. In some embodiments, providing a therapeutic regimen comprises: (a) determining that the patient has a normal or low level of meta-tyramine or a metabolic derivative thereof; and (b) providing a levodopa therapy lacking a tyrosine decarboxylase inhibitor to the patient.

In some embodiments, the present disclosure provides a method of providing a therapeutic regimen, comprising: (a) determining that a Parkinson's disease patient has an elevated level of meta-tyramine or a metabolic derivative thereof, or determining that a Parkinson's disease patient has a normal or low level of meta-tyramine or a metabolic derivative thereof; and (b) providing a levodopa therapy comprising a tyrosine decarboxylase inhibitor to the Parkinson's disease patient who has an elevated level of meta-tyramine or a metabolic derivative thereof, or providing a levodopa therapy lacking a tyrosine decarboxylase inhibitor to the Parkinson's disease patient who has a normal or low level of meta-tyramine or a metabolic derivative thereof. In some embodiments, the present disclosure provides use of meta-tyramine or a metabolic derivative thereof as a biomarker for providing a therapeutic regimen. In some embodiments, the present disclosure provides use of meta-tyramine or a metabolic derivative thereof as a biomarker in the manufacture of a medicament for providing a therapeutic regimen. In some embodiments, the present disclosure provides meta-tyramine or a metabolic derivative thereof for use as a biomarker for providing a therapeutic regimen. In some embodiments, providing a therapeutic regimen comprises: (a) determining that a Parkinson's disease patient has an elevated level of meta-tyramine or a metabolic derivative thereof, or determining that a Parkinson's disease patient has a normal or low level of meta-tyramine or a metabolic derivative thereof; and (b) providing a levodopa therapy comprising a tyrosine decarboxylase inhibitor to the Parkinson's disease patient who has an elevated level of meta-tyramine or a metabolic derivative thereof, or providing a levodopa therapy lacking a tyrosine decarboxylase inhibitor to the Parkinson's disease patient who has a normal or low level of meta-tyramine or a metabolic derivative thereof.

In some embodiments of the methods and uses disclosed herein, the method or use further comprises obtaining a biological sample from the patient, and determining the level of meta-tyramine or a metabolic derivative thereof in the sample.

In some embodiments, the present disclosure provides a method of treating Parkinson's disease in a patient in need thereof, comprising: (a) obtaining a biological sample from the patient; (b) determining from the sample that the patient has an elevated level of meta-tyramine or a metabolic derivative thereof; and (c) administering a levodopa therapy comprising a tyrosine decarboxylase inhibitor to the patient. In some embodiments, the present disclosure provides use of meta-tyramine or a metabolic derivative thereof as a biomarker for treating Parkinson's disease in a patient in need thereof. In some embodiments, the present disclosure provides use of meta-tyramine or a metabolic derivative thereof as a biomarker in the manufacture of a medicament for treating Parkinson's disease in a patient in need thereof. In some embodiments, the present disclosure provides meta-tyramine or a metabolic derivative thereof for use as a biomarker for treating Parkinson's disease in a patient in need thereof. In some embodiments, treating comprises: (a) obtaining a biological sample from the patient; (b) determining from the sample that the patient has an elevated level of meta-tyramine or a metabolic derivative thereof; and (c) administering a levodopa therapy comprising a tyrosine decarboxylase inhibitor to the patient.

In some embodiments, the present disclosure provides a method of treating Parkinson's disease in a patient in need thereof, comprising: (a) obtaining a biological sample from the patient; (b) determining from the sample that the patient has a normal or low level of meta-tyramine or a metabolic derivative thereof; and (c) administering a levodopa therapy lacking a tyrosine decarboxylase inhibitor to the patient. In some embodiments, the present disclosure provides use of meta-tyramine or a metabolic derivative thereof as a biomarker for treating Parkinson's disease in a patient in need thereof. In some embodiments, the present disclosure provides use of meta-tyramine or a metabolic derivative thereof as a biomarker in the manufacture of a medicament for treating Parkinson's disease in a patient in need thereof. In some embodiments, the present disclosure provides meta-tyramine or a metabolic derivative thereof for use as a biomarker for treating Parkinson's disease in a patient in need thereof. In some embodiments, treating comprises: (a) obtaining a biological sample from the patient; (b) determining from the sample that the patient has a normal or low level of meta-tyramine or a metabolic derivative thereof; and (c) administering a levodopa therapy lacking a tyrosine decarboxylase inhibitor to the patient.

In some embodiments, the present disclosure provides a method of treating Parkinson's disease in a patient in need thereof, comprising: (a) obtaining a biological sample from the patient; (b) determining from the sample that the patient has an elevated level of meta-tyramine or a metabolic derivative thereof, or determining from the sample that the patient has a normal or low level of meta-tyramine or a metabolic derivative thereof; and (c) administering a levodopa therapy comprising a tyrosine decarboxylase inhibitor to the patient if the patient has an elevated level of meta-tyramine or a metabolic derivative thereof, or administering a levodopa therapy lacking a tyrosine decarboxylase inhibitor to the patient if the patient has a normal or low level of meta-tyramine or a metabolic derivative thereof. In some embodiments, the present disclosure provides use of meta-tyramine or a metabolic derivative thereof as a biomarker for treating Parkinson's disease in a patient in need thereof. In some embodiments, the present disclosure provides use of meta-tyramine or a metabolic derivative thereof as a biomarker in the manufacture of a medicament for treating Parkinson's disease in a patient in need thereof. In some embodiments, the present disclosure provides meta-tyramine or a metabolic derivative thereof for use as a biomarker for treating Parkinson's disease in a patient in need thereof. In some embodiments, treating comprises: (a) obtaining a biological sample from the patient; (b) determining from the sample that the patient has an elevated level of meta-tyramine or a metabolic derivative thereof, or determining from the sample that the patient has a normal or low level of meta-tyramine or a metabolic derivative thereof; and (c) administering a levodopa therapy comprising a tyrosine decarboxylase inhibitor to the patient if the patient has an elevated level of meta-tyramine or a metabolic derivative thereof, or administering a levodopa therapy lacking a tyrosine decarboxylase inhibitor to the patient if the patient has a normal or low level of meta-tyramine or a metabolic derivative thereof.

In some embodiments, the present disclosure provides a method of identifying a suitable levodopa therapy for a Parkinson's disease patient, the method comprising: (a) obtaining a biological sample from the patient; (b) determining from the sample that the patient has an elevated level of meta-tyramine or a metabolic derivative thereof; and (c) identifying a levodopa therapy comprising a tyrosine decarboxylase inhibitor as a suitable levodopa therapy for the patient. In some embodiments, the present disclosure provides use of meta-tyramine or a metabolic derivative thereof as a biomarker for identifying a suitable levodopa therapy for a Parkinson's disease patient. In some embodiments, the present disclosure provides use of meta-tyramine or a metabolic derivative thereof as a biomarker in the manufacture of a medicament for identifying a suitable levodopa therapy for a Parkinson's disease patient. In some embodiments, the present disclosure provides meta-tyramine or a metabolic derivative thereof for use as a biomarker for identifying a suitable levodopa therapy for a Parkinson's disease patient. In some embodiments, identifying a suitable levodopa therapy for a Parkinson's disease patient comprises: (a) obtaining a biological sample from the patient; (b) determining from the sample that the patient has an elevated level of meta-tyramine or a metabolic derivative thereof; and (c) identifying a levodopa therapy comprising a tyrosine decarboxylase inhibitor as a suitable levodopa therapy for the patient.

In some embodiments, the present disclosure provides a method of identifying a suitable levodopa therapy for a Parkinson's disease patient, the method comprising: (a) obtaining a biological sample from the patient; (b) determining from the sample that the patient has a normal or low level of meta-tyramine or a metabolic derivative thereof; and (c) identifying a levodopa therapy lacking a tyrosine decarboxylase inhibitor as a suitable levodopa therapy for the patient. In some embodiments, the present disclosure provides use of meta-tyramine or a metabolic derivative thereof as a biomarker for identifying a suitable levodopa therapy for a Parkinson's disease patient. In some embodiments, the present disclosure provides use of meta-tyramine or a metabolic derivative thereof as a biomarker in the manufacture of a medicament for identifying a suitable levodopa therapy for a Parkinson's disease patient. In some embodiments, the present disclosure provides meta-tyramine or a metabolic derivative thereof for use as a biomarker for identifying a suitable levodopa therapy for a Parkinson's disease patient. In some embodiments, identifying a suitable levodopa therapy for a Parkinson's disease patient comprises: (a) obtaining a biological sample from the patient; (b) determining from the sample that the patient has a normal or low level of meta-tyramine or a metabolic derivative thereof; and (c) identifying a levodopa therapy lacking a tyrosine decarboxylase inhibitor as a suitable levodopa therapy for the patient.

In some embodiments, the present disclosure provides a method of identifying a suitable levodopa therapy for a Parkinson's disease patient, the method comprising: (a) obtaining a biological sample from the patient; (b) determining from the sample that the patient has an elevated level of meta-tyramine or a metabolic derivative thereof, or determining from the sample that the patient has a normal or low level of meta-tyramine or a metabolic derivative thereof; and (c) identifying a levodopa therapy comprising a tyrosine decarboxylase inhibitor as a suitable levodopa therapy for the patient if the patient has an elevated level of meta-tyramine or a metabolic derivative thereof, or identifying a levodopa therapy lacking a tyrosine decarboxylase inhibitor as a suitable levodopa therapy for the patient if the patient has a normal or low level of meta-tyramine or a metabolic derivative thereof. In some embodiments, the present disclosure provides use of meta-tyramine or a metabolic derivative thereof as a biomarker for identifying a suitable levodopa therapy for a Parkinson's disease patient. In some embodiments, the present disclosure provides use of meta-tyramine or a metabolic derivative thereof as a biomarker in the manufacture of a medicament for identifying a suitable levodopa therapy for a Parkinson's disease patient. In some embodiments, the present disclosure provides meta-tyramine or a metabolic derivative thereof for use as a biomarker for identifying a suitable levodopa therapy for a Parkinson's disease patient. In some embodiments, identifying a suitable levodopa therapy for a Parkinson's disease patient comprises: (a) obtaining a biological sample from the patient; (b) determining from the sample that the patient has an elevated level of meta-tyramine or a metabolic derivative thereof, or determining from the sample that the patient has a normal or low level of meta-tyramine or a metabolic derivative thereof; and (c) identifying a levodopa therapy comprising a tyrosine decarboxylase inhibitor as a suitable levodopa therapy for the patient if the patient has an elevated level of meta-tyramine or a metabolic derivative thereof, or identifying a levodopa therapy lacking a tyrosine decarboxylase inhibitor as a suitable levodopa therapy for the patient if the patient has a normal or low level of meta-tyramine or a metabolic derivative thereof.

In some embodiments of the methods and uses disclosed herein, the biological sample comprises a plasma sample, a urine sample, a stool sample, an intestinal sample, or a combination thereof. In some embodiments, the biological sample comprises a plasma sample, a urine sample, and/or an intestinal sample. In some embodiments, the biological sample comprises a plasma sample and a urine sample. In some embodiments, the biological sample comprises a plasma sample. In some embodiments, the plasma sample comprises peripheral blood plasma. In some embodiments, the biological sample comprises an intestinal sample. In some embodiments, the biological sample comprises an intestinal sample from the duodenum, the jejunum, the ileum, the ascending colon, the descending colon, and/or the transverse colon. In some embodiments, the intestinal sample is from the lower intestine (e.g., the ascending colon, the descending colon, and/or the transverse colon).

In some embodiments, the biological sample comprises a sample (e.g., a plasma sample, a urine sample, and/or an intestinal sample) from a single subject. In some embodiments, the biological sample comprises one or more longitudinal samples, i.e., samples collected from a single subject at different points in time. In some embodiments, the biological sample is from a subject who is receiving a known levodopa regimen. In some embodiments, the biological sample is from a subject who is receiving a known levodopa regimen and there is a known timing between the last dose of levodopa and the sample collection. In some embodiments, the biological sample is from a healthy subject. In some embodiments, the biological sample is from a Parkinson's disease patient.

In some embodiments, the biological sample comprises at least 0.1 mL, at least 0.25 mL, at least 0.5 mL, at least 0.75 mL, at least 1 mL, at least 1.5 mL, at least 2 mL, at least 2.5 mL, or at least 3 mL of each sample type (e.g., a plasma sample, a urine sample, etc.). In some embodiments, the biological sample comprises at least 1 mL of each sample type (e.g., a plasma sample, a urine sample, etc.).

In some embodiments, the biological sample comprises a plasma sample. In some embodiments, the plasma sample provides a representative snapshot of a subject's microbial metabolism of levodopa. In some embodiments, this snapshot may be used to assess and/or quantify the impact of the microbiome on levodopa. In some embodiments, the plasma sample provides one or more advantages over other sample types (e.g., a urine sample), e.g., by reducing or eliminating variability due to hydration level and/or urination frequency.

In some embodiments, the biological sample comprises a urine sample. In some embodiments, the urine sample provides one or more advantages over other sample types (e.g., a plasma sample), e.g., by allowing the detection of compounds that only accumulate to low levels in a subject and/or are rapidly cleared from a subject.

In some embodiments, the biological sample comprises a plasma sample and a urine sample. In some embodiments, the biological sample comprising a plasma sample and a urine sample provides one or more advantages over other sample types or combinations thereof, e.g., by establishing a metabolic map of all transformations (e.g., due to the accumulative nature of urine that may amplify signals). In some embodiments, using a urine sample in combination with a plasma sample enables an additional level of characterization because urine is known to harbor discriminating signals between affected patients (e.g., Parkinson's disease patients) and controls (Michell et al., Metabolomics 2008; 4:191-201; Tropini et al., Cell Host Microbe 2017; 21(4):433-442). In some embodiments, this paired sample approach may provide both a quantitative instantaneous view of microbial metabolism of levodopa from the plasma, as well as a qualitative overview of the products that accumulate in the urine over time.

In some embodiments, the biological sample comprises at least 1 mL of a plasma sample. In some embodiments, the biological sample comprises at least 1 mL of a urine sample. In some embodiments, the biological sample comprises at least 1 mL of a plasma sample and at least 1 mL of a urine sample. In some embodiments, the biological sample comprises at least 1 mL of a plasma sample and at least 1 mL of a urine sample from a healthy subject. In some embodiments, the biological sample comprises at least 1 mL of a plasma sample and at least 1 mL of a urine sample from a Parkinson's disease patient.

In some embodiments of the methods and uses disclosed herein, the patient (e.g., a Parkinson's disease patient) is receiving a levodopa therapy lacking a tyrosine decarboxylase inhibitor.

In some embodiments, the level of meta-tyramine or a metabolic derivative thereof is determined less than about 15 hours, less than about 12 hours, less than about 10 hours, less than about 8 hours, less than about 6 hours, less than about 5 hours, less than about 4 hours, less than about 3 hours, less than about 2 hours, or less than about 1 hour after the patient is administered a single dose of the levodopa therapy lacking a tyrosine decarboxylase inhibitor. In some embodiments, the level of meta-tyramine or a metabolic derivative thereof is determined less than about 6 hours, less than about 5.5 hours, less than about 5 hours, less than about 4.5 hours, less than about 4 hours, less than about 3.5 hours, less than about 3 hours, less than about 2.5 hours, less than about 2 hours, less than about 1.5 hours, or less than about 1 hour (e.g., about 15, 30, or 45 minutes) after the patient is administered a single dose of the levodopa therapy lacking a tyrosine decarboxylase inhibitor. In some embodiments, the level of meta-tyramine or a metabolic derivative thereof is determined less than about 5 hours after the patient is administered a single dose of the levodopa therapy lacking a tyrosine decarboxylase inhibitor. In some embodiments, the level of meta-tyramine or a metabolic derivative thereof is determined less than about 4 hours after the patient is administered a single dose of the levodopa therapy lacking a tyrosine decarboxylase inhibitor. In some embodiments, the level of meta-tyramine or a metabolic derivative thereof is determined less than about 3 hours after the patient is administered a single dose of the levodopa therapy lacking a tyrosine decarboxylase inhibitor. In some embodiments, the level of meta-tyramine or a metabolic derivative thereof is determined less than about 2 hours after the patient is administered a single dose of the levodopa therapy lacking a tyrosine decarboxylase inhibitor. In some embodiments, the level of meta-tyramine or a metabolic derivative thereof is determined less than about 1 hour (e.g., about 15, 30, or 45 minutes) after the patient is administered a single dose of the levodopa therapy lacking a tyrosine decarboxylase inhibitor.

In some embodiments, the level of meta-tyramine or a metabolic derivative thereof is determined about 0.25 to about 6 hours, about 1 to about 5 hours, about 1 to about 4 hours, about 1 to about 3 hours, about 1 to about 2 hours, or about 1 hour or less after the patient is administered a single dose of the levodopa therapy lacking a tyrosine decarboxylase inhibitor. In some embodiments, the level of meta-tyramine or a metabolic derivative thereof is determined about 1 to about 3 hours after the patient is administered a single dose of the levodopa therapy lacking a tyrosine decarboxylase inhibitor. In some embodiments, the level of meta-tyramine or a metabolic derivative thereof is determined about 1, about 1.5, about 2, about 2.5, or about 3 hours after the patient is administered a single dose of the levodopa therapy lacking a tyrosine decarboxylase inhibitor.

In some embodiments of the methods and uses disclosed herein, the level of meta-tyramine or a metabolic derivative thereof is measured by metabolomics or enzyme-linked immunosorbent assay (ELISA).

In some embodiments, the metabolomics is performed on a biological sample comprising one or more than one sample type. For instance, in some embodiments, the metabolomics is performed on paired plasma and urine samples. In some embodiments, one or more of the metabolites detected in the biological sample are quantified, e.g., using authentic standards. In some embodiments, a metabolic map of one or more of the metabolites detected in the biological sample and originating from the microbial metabolism of levodopa are compiled. In some embodiments, this compilation (e.g., together with the measured level (e.g., concentration) of each metabolite) is used to estimate the extent of levodopa metabolism by the microbiome in each patient. In some embodiments, when paired plasma and urine samples are used, the composition of plasma may be relatively comparable between samples with regard to concentration, however, the urine samples may span a range of concentrations due to differences in levels of hydration and frequency of urination between patients. In some embodiments, to harmonize the results from these different biofluid types, plasma samples may be used for quantitative measures and urine samples may provide observational support.

In some embodiments, the metabolomics comprises liquid chromatography-mass spectrometry (LC-MS), gas-phase chromatography-mass spectrometry (GC-MS), or tandem mass spectrometry (MS-MS). In some embodiments, GC-MS enables sensitive detection of compounds. In some embodiments, GC-MS provides greater sensitivity than alternate metabolomics platforms, such as LC-MS.

In some embodiments, the metabolomics comprises reversed-phase chromatography with positive ionization mode, reversed-phase chromatography with negative ionization mode, hydrophobic interaction liquid ion chromatography (HILIC) with positive ionization mode, hydrophobic interaction liquid ion chromatography (HILIC) with negative ionization mode, or a combination thereof. In some embodiments, the metabolomics comprises a combination of reversed-phase chromatography with positive ionization mode, reversed-phase chromatography with negative ionization mode, HILIC with positive ionization mode, and HILIC with negative ionization mode.

In some embodiments of the methods and uses disclosed herein, meta-tyramine or a metabolic derivative thereof is differentially abundant between samples and/or sample cohorts. In some embodiments, meta-tyramine or a metabolic derivative thereof is differentially abundant between the patient or patient cohort and a control or control cohort. In some embodiments, the patient or patient cohort is a Parkinson's disease patient, or a group of two or more Parkinson's disease patients (e.g., a group of about 5, 10, 18, 20, 30, 40, 50, 60, 70, 75, or more Parkinson's disease patients). In some embodiments, the control or control cohort is a healthy subject naïve to levodopa, or a group of two or more healthy subjects naïve to levodopa (e.g., a group of about 5, 10, 18, 20, 30, 40, 50, 60, 70, 75, or more healthy subjects naïve to levodopa). In some embodiments, the control or control cohort is a Parkinson's disease patient naïve to levodopa or not currently on a levodopa therapy, or a group of two or more Parkinson's disease patients naïve to levodopa or not currently on a levodopa therapy (e.g., a group of about 5, 10, 18, 20, 30, 40, 50, 60, 70, 75, or more Parkinson's disease patients naïve to levodopa or not currently on a levodopa therapy).

In some embodiments, the presence and/or level of meta-tyramine or a metabolic derivative thereof differs between samples and/or sample cohorts, as determined using one or more statistical tests with a set significance threshold. In some embodiments, a difference in the presence and/or level of meta-tyramine or a metabolic derivative thereof between samples and/or sample cohorts is determined using at least two different statistical tests, e.g., to reduce the possibility of analytical bias.

As used herein, the term “elevated level” when used to describe the level of meta-tyramine or a metabolic derivative thereof in a patient, patient cohort, or patient sample, means a level exceeding (i.e., higher than) the level of meta-tyramine or a metabolic derivative thereof in a control, control cohort, or control sample. In some embodiments, the patient or patient cohort is a Parkinson's disease patient, or a group of two or more Parkinson's disease patients (e.g., a group of about 5, 10, 18, 20, 30, 40, 50, 60, 70, 75, or more Parkinson's disease patients). In some embodiments, the control or control cohort is a healthy subject naïve to levodopa, or a group of two or more healthy subjects naïve to levodopa (e.g., a group of about 5, 10, 18, 20, 30, 40, 50, 60, 70, 75, or more healthy subjects naïve to levodopa). In some embodiments, the control or control cohort is a Parkinson's disease patient naïve to levodopa or not currently on a levodopa therapy, or a group of two or more Parkinson's disease patients naïve to levodopa or not currently on a levodopa therapy (e.g., a group of about 5, 10, 18, 20, 30, 40, 50, 60, 70, 75, or more Parkinson's disease patients naïve to levodopa or not currently on a levodopa therapy).

In some embodiments, an elevated level of meta-tyramine or a metabolic derivative thereof in a patient is a level exceeding the level in a healthy subject naïve to levodopa (e.g., a level that is at least about 10%, 20%, 30%, 40%, 50%, 60%, 70%, 80%, 90%, or 100% higher than the level in a healthy subject naïve to levodopa). In some embodiments, an elevated level of meta-tyramine or a metabolic derivative thereof in a patient is a level exceeding the level in a Parkinson's disease patient naïve to levodopa or not currently on a levodopa therapy (e.g., a level that is at least about 10%, 20%, 30%, 40%, 50%, 60%, 70%, 80%, 90%, or 100% higher than the level in a Parkinson's disease patient naïve to levodopa or not currently on a levodopa therapy). In some embodiments, an elevated level of meta-tyramine or a metabolic derivative thereof in a patient is a level exceeding the level in a Parkinson's disease patient or patient population that is currently on and responsive to a levodopa therapy (e.g., a level that is at least about 10%, 20%, 30%, 40%, 50%, 60%, 70%, 80%, 90%, or 100% higher than the level in a Parkinson's disease patient or patient population that is currently on and responsive to a levodopa therapy). In some embodiments, an elevated level of meta-tyramine or a metabolic derivative thereof in a patient is a level exceeding 100 ng/mL.

As used herein, the term “normal or low level” when used to describe the level of meta-tyramine or a metabolic derivative thereof in a patient, patient cohort, or patient sample, means a level equal to or below (i.e., the same or lower than) the level of meta-tyramine or a metabolic derivative thereof in a control, control cohort, or control sample. In some embodiments, the patient or patient cohort is a Parkinson's disease patient or a group of two or more Parkinson's disease patients (e.g., a group of about 5, 10, 18, 20, 30, 40, 50, 60, 70, 75, or more Parkinson's disease patients). In some embodiments, the control or control cohort is a healthy subject naïve to levodopa, or a group of two or more healthy subjects naïve to levodopa (e.g., a group of about 5, 10, 18, 20, 30, 40, 50, 60, 70, 75, or more healthy subjects naïve to levodopa). In some embodiments, the control or control cohort is a Parkinson's disease patient naïve to levodopa or not currently on a levodopa therapy, or a group of two or more Parkinson's disease patients naïve to levodopa or not currently on a levodopa therapy (e.g., a group of about 5, 10, 18, 20, 30, 40, 50, 60, 70, 75, or more Parkinson's disease patients naïve to levodopa or not currently on a levodopa therapy).

In some embodiments, a normal or low level of meta-tyramine or a metabolic derivative thereof in a patient is a level equal to or below the level in a healthy subject naïve to levodopa (e.g., a level that is equal to or at least about 10%, 20%, 30%, 40%, 50%, 60%, 70%, 80%, 90%, or 100% lower than the level in a healthy subject naïve to levodopa). In some embodiments, a normal or low level of meta-tyramine or a metabolic derivative thereof in a patient is a level equal to or below the level in a Parkinson's disease patient naïve to levodopa or not currently on a levodopa therapy (e.g., a level that is equal to or at least about 10%, 20%, 30%, 40%, 50%, 60%, 70%, 80%, 90%, or 100% lower than the level in a Parkinson's disease patient naïve to levodopa or not currently on a levodopa therapy). In some embodiments, a normal or low level of meta-tyramine or a metabolic derivative thereof in a patient is a level equal to or below the level in a Parkinson's disease patient or patient population that is currently on and responsive to a levodopa therapy (e.g., a level that is equal to or at least about 10%, 20%, 30%, 40%, 50%, 60%, 70%, 80%, 90%, or 100% lower than the level in a Parkinson's disease patient or patient population that is currently on and responsive to a levodopa therapy). In some embodiments, a normal or low level of meta-tyramine or a metabolic derivative thereof in the patient is a level equal to or below 100 ng/mL.

In some embodiments of the methods and uses disclosed herein, the levodopa is administered simultaneously with the tyrosine decarboxylase inhibitor. In some embodiments, the levodopa is administered sequentially with the tyrosine decarboxylase inhibitor. In some embodiments, the levodopa therapy comprising a tyrosine decarboxylase inhibitor results in an increased level of circulating levodopa compared to the level of circulating levodopa prior to treatment.

In some embodiments, the amount of levodopa administered in combination with the tyrosine decarboxylase inhibitor is reduced compared to the amount of levodopa administered in the absence of the tyrosine decarboxylase inhibitor. In some embodiments, the amount of levodopa administered in combination with the tyrosine decarboxylase inhibitor is reduced by at least 10%, 20%, 30%, 40%, 50%, 60%, 70%, or 80% or more compared to the amount of levodopa administered in the absence of the tyrosine decarboxylase inhibitor. In some embodiments, the amount of levodopa administered in combination with the tyrosine decarboxylase inhibitor is reduced by at least 10% compared to the amount of levodopa administered in the absence of the tyrosine decarboxylase inhibitor. In some embodiments, the amount of levodopa administered in combination with the tyrosine decarboxylase inhibitor is reduced by at least 20% compared to the amount of levodopa administered in the absence of the tyrosine decarboxylase inhibitor. In some embodiments, the amount of levodopa administered in combination with the tyrosine decarboxylase inhibitor is reduced by at least 30% compared to the amount of levodopa administered in the absence of the tyrosine decarboxylase inhibitor. In some embodiments, the amount of levodopa administered in combination with the tyrosine decarboxylase inhibitor is reduced by at least 40% compared to the amount of levodopa administered in the absence of the tyrosine decarboxylase inhibitor. In some embodiments, the amount of levodopa administered in combination with the tyrosine decarboxylase inhibitor is reduced by at least 50% compared to the amount of levodopa administered in the absence of the tyrosine decarboxylase inhibitor. In some embodiments, the amount of levodopa administered in combination with the tyrosine decarboxylase inhibitor is reduced by at least 60% compared to the amount of levodopa administered in the absence of the tyrosine decarboxylase inhibitor. In some embodiments, the amount of levodopa administered in combination with the tyrosine decarboxylase inhibitor is reduced by at least 70% compared to the amount of levodopa administered in the absence of the tyrosine decarboxylase inhibitor. In some embodiments, the amount of levodopa administered in combination with the tyrosine decarboxylase inhibitor is reduced by at least 80% compared to the amount of levodopa administered in the absence of the tyrosine decarboxylase inhibitor. In some embodiments, the amount of levodopa administered in combination with the tyrosine decarboxylase inhibitor is reduced by more than 80% (e.g., 90%, 95%, etc.) compared to the amount of levodopa administered in the absence of the tyrosine decarboxylase inhibitor.

In some embodiments, the levodopa administered in combination with the tyrosine decarboxylase inhibitor is administered less frequently compared to the levodopa administered in the absence of the tyrosine decarboxylase inhibitor. In some embodiments, the levodopa administered in combination with the tyrosine decarboxylase inhibitor is administered at least 10%, 20%, 30%, 40%, 50%, 60%, 70%, or 80% less frequently or less compared to the levodopa administered in the absence of the tyrosine decarboxylase inhibitor. In some embodiments, the levodopa administered in combination with the tyrosine decarboxylase inhibitor is administered at least 10% less frequently compared to the levodopa administered in the absence of the tyrosine decarboxylase inhibitor. In some embodiments, the levodopa administered in combination with the tyrosine decarboxylase inhibitor is administered at least 20% less frequently compared to the levodopa administered in the absence of the tyrosine decarboxylase inhibitor. In some embodiments, the levodopa administered in combination with the tyrosine decarboxylase inhibitor is administered at least 30% less frequently compared to the levodopa administered in the absence of the tyrosine decarboxylase inhibitor. In some embodiments, the levodopa administered in combination with the tyrosine decarboxylase inhibitor is administered at least 40% less frequently compared to the levodopa administered in the absence of the tyrosine decarboxylase inhibitor. In some embodiments, the levodopa administered in combination with the tyrosine decarboxylase inhibitor is administered at least 50% less frequently compared to the levodopa administered in the absence of the tyrosine decarboxylase inhibitor. In some embodiments, the levodopa administered in combination with the tyrosine decarboxylase inhibitor is administered at least 60% less frequently compared to the levodopa administered in the absence of the tyrosine decarboxylase inhibitor. In some embodiments, the levodopa administered in combination with the tyrosine decarboxylase inhibitor is administered at least 70% less frequently compared to the levodopa administered in the absence of the tyrosine decarboxylase inhibitor. In some embodiments, the levodopa administered in combination with the tyrosine decarboxylase inhibitor is administered at least 80% less frequently compared to the levodopa administered in the absence of the tyrosine decarboxylase inhibitor. In some embodiments, the levodopa administered in combination with the tyrosine decarboxylase inhibitor is administered more than 80% (e.g., 90%, 95%, etc.) less frequently compared to the levodopa administered in the absence of the tyrosine decarboxylase inhibitor.

In some embodiments, the treatment with levodopa in combination with the tyrosine decarboxylase inhibitor results in reduced systemic toxicity and/or improved tolerance compared to the treatment with levodopa in the absence of the tyrosine decarboxylase inhibitor.

In some embodiments of the methods and uses disclosed herein, the levodopa therapy further comprises a peripheral aromatic amino acid decarboxylase inhibitor. In some embodiments, the peripheral aromatic amino acid decarboxylase inhibitor is carbidopa.

In some embodiments, the meta-tyramine or a metabolic derivative thereof comprises meta-tyramine, 3-hydroxyphenylacetic acid, 3-hydroxyphenylacetaldehyde, 3-hydroxyphenylacetate methyl ester, 3-sulfooxyphenylacetic acid, 3-methoxyphenylacetic acid, 3-methoxyphenethylamine, 3-hydroxyphenylethanol, 3-hydroxymandelic acid, meta-octopamine, meta-tyramine-O-sulfate, and/or meta-tyramine-O-glucuronide. In some embodiments, the meta-tyramine or a metabolic derivative thereof comprises meta-tyramine, 3-hydroxyphenylacetic acid, 3-hydroxyphenylacetate methyl ester, 3-sulfooxyphenylacetic acid, 3-methoxyphenylacetic acid, 3-methoxyphenethylamine, and/or meta-tyramine-O-sulfate. In some embodiments, the meta-tyramine or a metabolic derivative thereof comprises 3-hydroxyphenylacetic acid, 3-hydroxyphenylacetate methyl ester, 3-sulfooxyphenylacetic acid, and/or meta-tyramine-O-sulfate.

Therapeutic Compositions

Metabolism of levodopa by tyrosine decarboxylase may be inhibited using any of the exemplary tyrosine decarboxylase inhibitors described and/or incorporated by reference herein. In some embodiments, inhibition of tyrosine decarboxylase may lead to the modulation of one or more tyrosine decarboxylase markers. The tyrosine decarboxylase marker can be, for example, levodopa levels. In some embodiments, treatment with a tyrosine decarboxylase inhibitor increases the level of levodopa in a patient (e.g., a Parkinson's disease patient).

In some embodiments, a levodopa therapy comprising a tyrosine decarboxylase inhibitor is administered to a Parkinson's disease patient who has an elevated level of meta-tyramine or a metabolic derivative thereof. In some embodiments, levodopa and a tyrosine decarboxylase inhibitor are administered to the patient. In some embodiments, levodopa and a tyrosine decarboxylase inhibitor are administered to the patient in combination with one or more additional therapeutic agents. In some embodiments, levodopa and a tyrosine decarboxylase inhibitor are administered to the patient in combination with a peripheral aromatic amino acid decarboxylase inhibitor. In some embodiments, the peripheral aromatic amino acid decarboxylase inhibitor is carbidopa.

Administered “in combination” or “co-administration,” as used herein, means that two or more different treatments are delivered to a patient during the patient's affliction with a disease, disorder, or condition (e.g., Parkinson's disease). For example, in some embodiments, the two or more treatments are delivered after the patient has been diagnosed with a disease or disorder, and before the disease or disorder has been cured or eliminated. In some embodiments, the delivery of one treatment is still occurring when the delivery of the second treatment begins, so that there is overlap. In some embodiments, the first and second treatment are initiated at the same time. These types of delivery are sometimes referred to herein as “simultaneous,” “concurrent,” or “concomitant” delivery. In other embodiments, the delivery of one treatment ends before delivery of the second treatment begins. This type of delivery is sometimes referred to herein as “successive” or “sequential” delivery. In some embodiments, levodopa and a tyrosine decarboxylase inhibitor are administered simultaneously. In some embodiments, levodopa and a tyrosine decarboxylase inhibitor are administered sequentially. In either case, the two treatments should be administered sufficiently close in time so as to provide the desired therapeutic effect.

In some embodiments of simultaneous administration, the two treatments (e.g., levodopa and a tyrosine decarboxylase inhibitor) are comprised in the same formulation. Such formulations may be administered in any appropriate form and by any suitable route. In some embodiments, the two treatments (e.g., levodopa and a tyrosine decarboxylase inhibitor) are comprised in a mixture. In some embodiments, the two treatments comprise levodopa and a tyrosine decarboxylase inhibitor.

In other embodiments of simultaneous administration, the two treatments (e.g., levodopa and a tyrosine decarboxylase inhibitor) are administered as separate formulations, in any appropriate form and by any suitable route. In some embodiments, the two treatments comprise levodopa and a tyrosine decarboxylase inhibitor.

In some embodiments, a levodopa therapy lacking a tyrosine decarboxylase inhibitor is administered to a Parkinson's disease patient who has a normal or low level of meta-tyramine or a metabolic derivative thereof. In some embodiments, levodopa is administered alone or in combination with one or more alternative additional therapeutic agents (i.e., additional therapeutic agents that do not comprise a tyrosine decarboxylase inhibitor). In some embodiments, levodopa is administered in combination with a peripheral aromatic amino acid decarboxylase inhibitor. In some embodiments, the peripheral aromatic amino acid decarboxylase inhibitor is carbidopa.

In some embodiments, levodopa and/or a tyrosine decarboxylase inhibitor is administered in combination with carbidopa (or another peripheral aromatic amino acid decarboxylase inhibitor). In some embodiments, when the levodopa is administered orally as a single agent, it is typically decarboxylated to dopamine in extracerebral tissues such that only a small portion of a given dose is transported unchanged to the central nervous system. Thus, in some embodiments, large doses of levodopa may be required for adequate therapeutic effect. In some embodiments, these doses may often be accompanied by nausea and other adverse reactions, some of which are attributable to dopamine formed in extracerebral tissues. In some embodiments, the incidence of levodopa-induced nausea and vomiting is reduced when carbidopa is used with levodopa compared to when levodopa is used without carbidopa. In some embodiments, this reduction in nausea and vomiting permits more rapid dosage titration.

In some embodiments, when its decarboxylase-inhibiting activity is limited primarily to extracerebral tissues, administration of carbidopa with levodopa makes more levodopa available for transport to the brain. In some embodiments, carbidopa reduces the amount of levodopa required to produce a given response. In some embodiments, carbidopa reduces the amount of levodopa required to produce a given response by at least 50%, at least 60%, at least 70%, at least 75%, or at least 80% or more (e.g., by about 85%, 90%, 95%, 98%, etc.). In some embodiments, carbidopa reduces the amount of levodopa required to produce a given response by about 75% (Lodosyn (carbidopa) [package insert]. Bridgewater, N.J.: Valeant Pharmaceuticals North America LLC; 2014). In some embodiments, carbidopa, when administered with levodopa, increases plasma levels and/or the plasma half-life of the levodopa.

In some embodiments, the levodopa and/or the tyrosine decarboxylase inhibitor is administered in combination with carbidopa alone. In some embodiments, the levodopa and/or the tyrosine decarboxylase inhibitor is administered in combination with carbidopa and one or more additional therapeutic agents (e.g., pyridoxine). For instance, supplemental pyridoxine (vitamin B₆) can be administered to patients receiving carbidopa and levodopa concomitantly or a fixed combination carbidopa-levodopa or carbidopa-levodopa extended release.

In some embodiments, the levodopa and/or the tyrosine decarboxylase inhibitor is administered to a patient in a biologically compatible form. In some embodiments, the levodopa and/or tyrosine decarboxylase inhibitor is formulated into a pharmaceutical composition. In some embodiments, a pharmaceutical composition comprises the levodopa and a physiologically acceptable excipient (e.g., a pharmaceutically acceptable excipient). In some embodiments, a pharmaceutical composition comprises the tyrosine decarboxylase inhibitor and a physiologically acceptable excipient (e.g., a pharmaceutically acceptable excipient). In some embodiments, a pharmaceutical composition comprises the levodopa, the tyrosine decarboxylase inhibitor, and a physiologically acceptable excipient (e.g., a pharmaceutically acceptable excipient).

The levodopa and/or the tyrosine decarboxylase inhibitor may be administered to a patient in a variety of forms depending on the selected route of administration, as will be understood by those skilled in the art. For human use, levodopa and/or a tyrosine decarboxylase inhibitor described herein can be administered alone or in admixture with a pharmaceutical carrier selected based on the intended route of administration and standard pharmaceutical practice. Pharmaceutical compositions for use in accordance with the present disclosure can be formulated in a conventional manner using one or more physiologically acceptable carriers having excipients and/or auxiliaries that facilitate processing of levodopa and/or a tyrosine decarboxylase inhibitor described herein into preparations which can be used pharmaceutically.

In making pharmaceutical compositions, in some embodiments, the active agent (e.g., levodopa and/or a tyrosine decarboxylase inhibitor) is mixed with an excipient, diluted by an excipient, or enclosed within such a carrier in the form of, for example, a capsule, sachet, paper, or other container. In some embodiments, when the excipient serves as a diluent, it can be a solid, semisolid, or liquid material (e.g., normal saline), which acts as a vehicle, carrier, or medium for the active agent. In some embodiments, compositions can be in the form of tablets, powders, lozenges, sachets, cachets, elixirs, suspensions, emulsions, solutions, syrups, or soft or hard gelatin capsules. As is known in the art, the type of diluent can vary depending upon the intended route of administration. In some embodiments, the resulting compositions can also include additional agents, e.g., preservatives.

In some embodiments, an excipient or carrier is selected on the basis of the route of administration. Suitable pharmaceutical carriers for use in pharmaceutical formulations, are described in Remington: The Science and Practice of Pharmacy, 21st Ed., Gennaro, Ed., Lippencott Williams & Wilkins (2005), and in the USP/NF (United States Pharmacopeia and the National Formulary). Examples of suitable excipients are lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. In some embodiments, formulations can additionally include: lubricating agents, e.g., talc, magnesium stearate, and mineral oil; wetting agents; emulsifying and suspending agents; preserving agents, e.g., methyl- and propylhydroxy-benzoates; sweetening agents; and flavoring agents. Other exemplary excipients are described in Handbook of Pharmaceutical Excipients, 6th Edition, Rowe et al., Eds., Pharmaceutical Press (2009).

The pharmaceutical compositions described herein can be manufactured in a conventional manner, e.g., by conventional mixing, dissolving, granulating, dragee-making, levigating, emulsifying, encapsulating, entrapping, or lyophilizing processes. Methods well known in the art for making formulations are found, for example, in Remington: The Science and Practice of Pharmacy, 21st Ed., Gennaro, Ed., Lippencott Williams & Wilkins (2005), and Encyclopedia of Pharmaceutical Technology, eds. J. Swarbrick and J. C. Boylan, 1988-1999, Marcel Dekker, New York. In general, proper formulation is dependent upon the route of administration chosen. The formulation and preparation of such compositions is known to those skilled in the art of pharmaceutical formulation. In preparing a formulation, a compound can be milled to provide the appropriate particle size prior to combining with the other ingredients. If the compound is substantially insoluble, it can be milled to a particle size of less than 200 mesh. If the compound is substantially water soluble, the particle size can be adjusted by milling to provide a substantially uniform distribution in the formulation, e.g., about 40 mesh.

The dosage of levodopa and/or a tyrosine decarboxylase inhibitor used in the methods described herein, or pharmaceutical compositions thereof, can vary depending on many factors, e.g., the pharmacodynamic properties of the compound; the mode of administration; the age, health, and weight of the recipient; the nature and extent of the symptoms; the frequency of the treatment, and the type of concurrent treatment, if any; and the clearance rate of the compound in the subject to be treated. One of skill in the art can determine the appropriate dosage based on the above factors. In some embodiments, levodopa and/or a tyrosine decarboxylase inhibitor used in the methods described herein may be administered initially in a suitable dosage that may be adjusted as required, depending on the clinical response. In general, a suitable daily dose of the levodopa and/or tyrosine decarboxylase inhibitor may be an amount of the compound(s) that is the lowest dose effective to produce a therapeutic effect. Such an effective dose will generally depend upon the factors described above.

Levodopa and/or a tyrosine decarboxylase inhibitor may be administered to the patient in a single dose or in multiple doses. In some embodiments, when multiple doses are administered, the doses may be separated from one another by, for example, 1-24 hours, 1-7 days, or 1-4 weeks. One or both of the compounds may be administered according to a schedule, or one or both of the compounds may be administered without a predetermined schedule. For any particular subject, specific dosage regimes should be adjusted over time according to the individual need and the professional judgment of the person administering or supervising the administration of the compositions.

Levodopa and/or a tyrosine decarboxylase inhibitor may be provided in a unit dosage form. In some embodiments, the unit dosage form may be an oral unit dosage form (e.g., a tablet, capsule, suspension, liquid solution, powder, crystals, lozenge, sachet, cachet, elixir, syrup, and the like) or a food product serving (e.g., the active agent may be included as a food additive or dietary ingredient). In some embodiments, the dosage form is designed for administration of at least one compound described herein. The attending physician may ultimately decide the appropriate amount and dosage regimen. An effective amount of a tyrosine decarboxylase inhibitor described herein may be, for example, a total daily dosage of, e.g., between 0.5 g and 5 g (e.g., 0.5 to 2.5 g). Alternatively, the dosage amount may be calculated using the body weight of the subject. In some embodiments, when daily dosages exceed 5 g/day, the dosage of the compound may be divided across two or three daily administration events.

In the methods of the disclosure, the time period during which multiple doses of levodopa and/or a tyrosine decarboxylase inhibitor are administered to a subject can vary. For example, in some embodiments, doses of the compound(s) are administered to a subject over a time period that is 1-7 days; 1-12 weeks; or 1-3 months. In other embodiments, doses of the compound(s) are administered to the subject over a time period that is, for example, 4-11 months or 1-30 years. In other embodiments, doses of the compound(s) are administered to a subject at the onset of symptoms. In any of these embodiments, the amount of a compound that is administered may vary during the time period of administration. In some embodiments, when a compound is administered daily, administration may occur, for example, 1, 2, 3, or 4 times per day.

In some embodiments, the levodopa and/or the tyrosine decarboxylase inhibitor is administered to a patient with a pharmaceutically acceptable diluent, carrier, or excipient, in unit dosage form. Conventional pharmaceutical practice may be employed to provide suitable formulations or compositions comprising the levodopa and/or the tyrosine carboxylase inhibitor, and to administer such compositions to subjects suffering from a disease, disorder, or condition (e.g., Parkinson's disease) and/or before the subject is symptomatic.

Exemplary routes of administration of the levodopa and/or the tyrosine decarboxylase inhibitor, or a pharmaceutical composition thereof, include oral, sublingual, buccal, transdermal, intradermal, intramuscular, parenteral, intravenous, intra-arterial, intracranial, subcutaneous, intraorbital, intraventricular, intraspinal, intraperitoneal, intranasal, inhalation, and topical administration. In some embodiments, one or both of the compounds is administered with a physiologically acceptable carrier (e.g., a pharmaceutically acceptable carrier). In some embodiments, one or both of the compounds is administered to a subject orally.

The pharmaceutical compositions described herein include those formulated for oral administration (“oral dosage forms”). Oral dosage forms can be, for example, in the form of tablets, capsules, a liquid solution or suspension, a powder, or liquid or solid crystals, which contain the active agent in a mixture with physiologically acceptable excipients (e.g., pharmaceutically acceptable excipients). These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other physiologically acceptable excipients (e.g., pharmaceutically acceptable excipients) can be colorants, flavoring agents, plasticizers, humectants, buffering agents, and the like.

Formulations for oral administration may also be presented as chewable tablets, as hard gelatin capsules where the active agent is mixed with an inert solid diluent (e.g., potato starch, lactose, microcrystalline cellulose, calcium carbonate, calcium phosphate or kaolin), or as soft gelatin capsules where the active agent is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Controlled release compositions for oral use may be constructed to release the active agent by controlling its dissolution and/or diffusion. Any of a number of strategies can be pursued in order to obtain controlled release and a targeted plasma concentration versus time profile. In some embodiments, controlled release may be obtained by appropriate selection of various formulation parameters and ingredients, including, e.g., various types of controlled release compositions and coatings. Examples include single or multiple unit tablet or capsule compositions, oil solutions, suspensions, emulsions, microcapsules, microspheres, nanoparticles, patches, and liposomes. In some embodiments, compositions include biodegradable, pH, and/or temperature-sensitive polymer coatings.

Dissolution or diffusion controlled release can be achieved by appropriate coating of a tablet, capsule, pellet, or granulate formulation of compounds, or by incorporating the active agent into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

The liquid forms in which the described compounds and compositions can be incorporated for administration orally include aqueous solutions, suitably flavored syrups, aqueous or oil suspensions, and flavored emulsions with edible oils, e.g., cottonseed oil, sesame oil, coconut oil, or peanut oil, as well as elixirs and similar pharmaceutical vehicles.

Other routes of administration of the levodopa and/or the tyrosine decarboxylase inhibitor, or a pharmaceutical composition thereof, include sublingual, buccal, transdermal, intradermal, intramuscular, parenteral, intravenous, intra-arterial, intracranial, subcutaneous, intraorbital, intraventricular, intraspinal, intraperitoneal, intranasal, inhalation, and topical administration. Any form of administration capable of delivering the compounds to a patient (e.g., a Parkinson's disease patient) and providing the desired therapeutic effect are contemplated by the present disclosure.

Tyrosine Decarboxylase Inhibitors

Compounds which may inhibit a decarboxylase-mediated conversion of levodopa to dopamine are described and may be used in the methods, uses, and compositions disclosed herein. In some embodiments, the decarboxylase is a tyrosine decarboxylase. In some embodiments, the decarboxylase is a tyrosine decarboxylase from Enterococcus faecalis. In some embodiments, the compound is a tyrosine decarboxylase inhibitor.

In some embodiments, the tyrosine decarboxylase inhibitor is alpha-fluoromethyltyrosine (AFMT). The structure of AFMT is as follows:

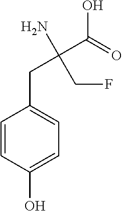 [SMILES: NC(CF)(Cc1ccc(O)cc1)C(=O)O]

In some embodiments, the tyrosine decarboxylase inhibitor is any of the exemplary compounds shown and described in PCT/US2019/064896, which is incorporated herein by reference for all its disclosed compounds and methods of synthesizing those compounds.

In some embodiments, the tyrosine decarboxylase inhibitor is a compound of formula (I):

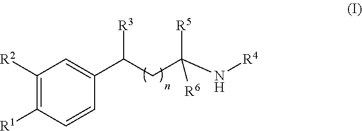

or a pharmaceutically acceptable salt thereof,
wherein

n is 0 or 1;

R is H or C1-6 alkyl;

R¹ is H or —OR^{A}, wherein R^{A} is H, —C(O)C₁₋₆alkyl, or an acylated sugar;

R² is H, halogen, amino, C₁₋₆ alkyl, or —OR^{A}, wherein R^{A} is H or an acylated sugar;

R³ is H, a halogen, —OH, or C₁₋₆ alkyl optionally substituted with one or more halogens;

R⁴ is H, —NH₂, —C(O)OCH₃, or an acylated sugar;

R⁵ is H, —C(O)OH, —C(O)OC₁₋₆ alkyl, —C(O)Oglycoside, —C(O)NHOH, or —C(O)O(acylated sugar); and

R⁶ is H, halogen, or optionally substituted C₁₋₆ alkyl;

provided that at least one R^{A} is present; or provided that R³ and/or R⁶ comprise a halogen.

In some embodiments the tyrosine decarboxylase inhibitor is a compound of formula (II):

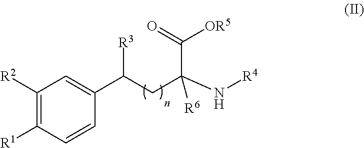

or a pharmaceutically acceptable salt thereof,
wherein

n is 0 or 1;

each of R¹ and R² is independently H or —OR^{A}, wherein each R^{A} is independently H or an acylated sugar, or R¹ is —C(O)C₁₋₆ alkyl;

R³ is H or a halogen;

R⁴ is H, —NH₂, —C(O)OCH₃, or an acylated sugar;

R⁵ is H, C₁₋₆ alkyl, glycoside, or an acylated sugar; and

R⁶ is H or optionally substituted C₁₋₆ alkyl;

provided that at least one R^{A} is present; or provided that R³ and/or R⁶ comprise a halogen.

In some embodiments, the tyrosine decarboxylase inhibitor is a compound of formula (I-a):

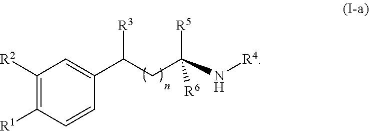

In some embodiments of formula (I) or (I-a), R¹ is —OR^{A}. In some embodiments, R² is H or —OR^{A}. In some embodiments, each R^{A} is H. In some embodiments, R³ is fluoro or chloro. In some embodiments, R⁴ is H. In some embodiments, R⁴ is —NH₂. In some embodiments, R⁵ is H. In some embodiments, R⁵ is an acylated sugar. In some embodiments, R⁶ is H. In some embodiments, R⁶ is alkyl. In some embodiments, n is 0. In some embodiments, n is 1. In some embodiments, R² is halogen. In some embodiments, R³ is a C₁₋₆ alkyl. In some embodiments, R⁵ is H. In some embodiments, R⁶ is halogen.

In some embodiments, the tyrosine decarboxylase inhibitor is a compound chosen from the following compounds:

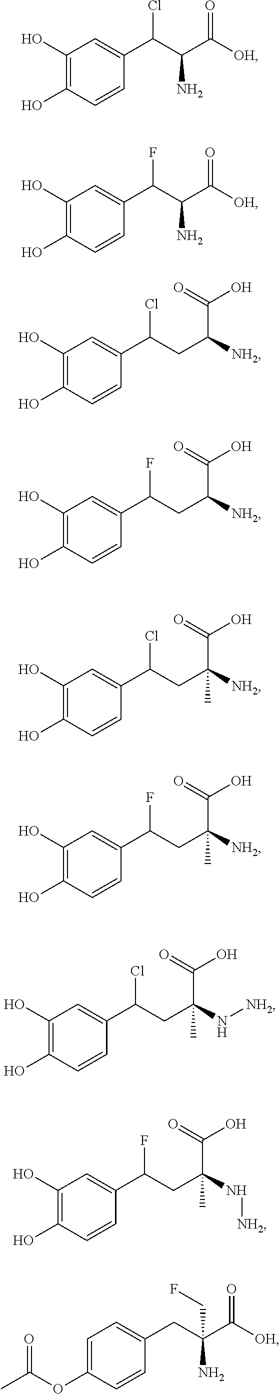

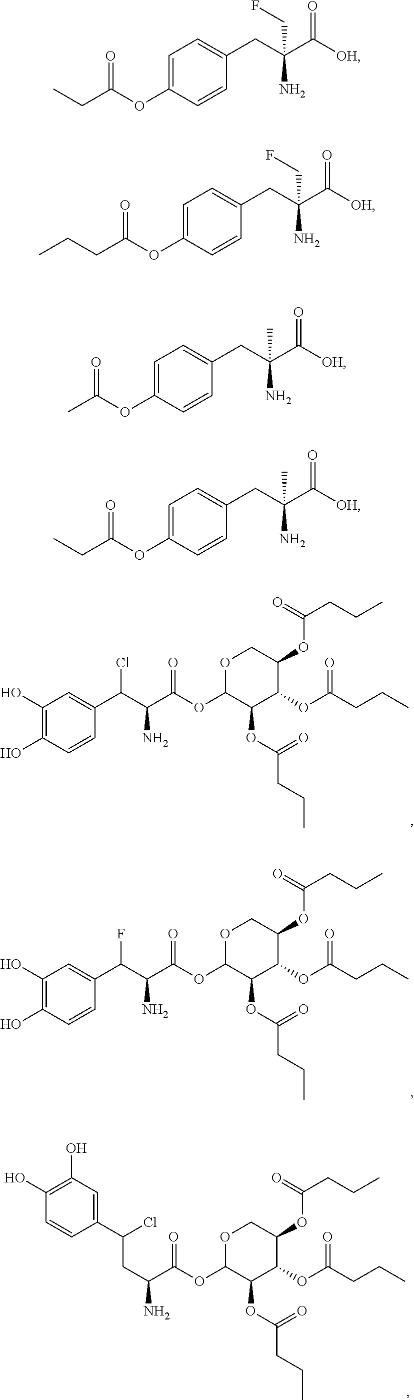

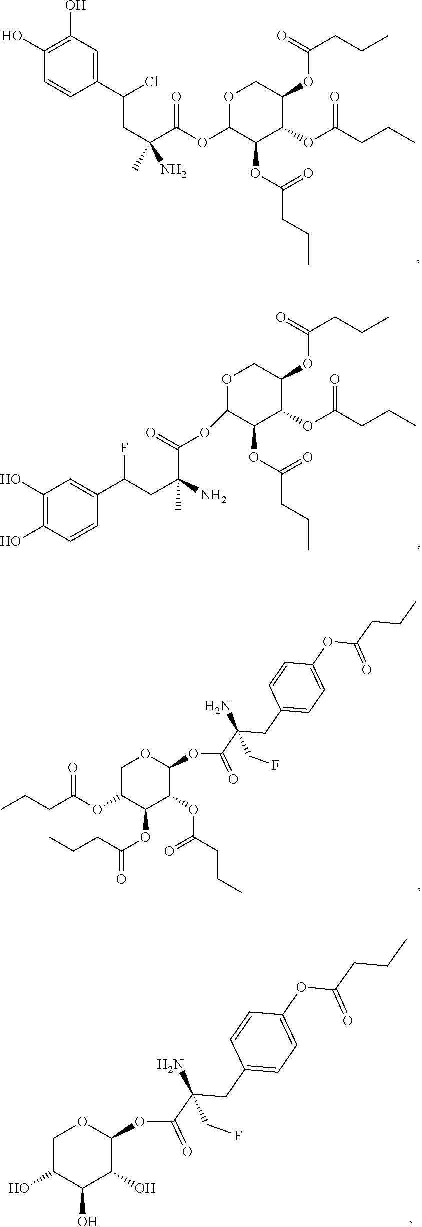

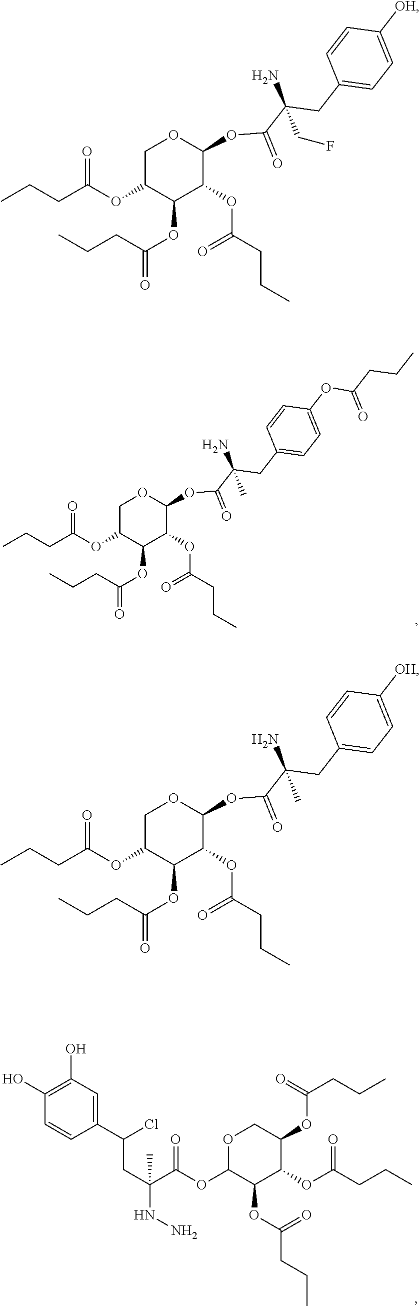

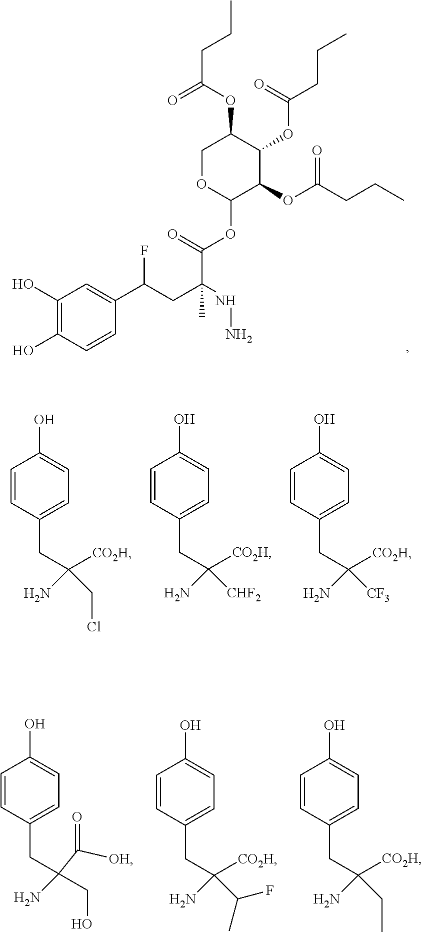

and pharmaceutically acceptable salts thereof.

In some embodiments, the tyrosine decarboxylase inhibitor is a compound chosen from the following compounds:

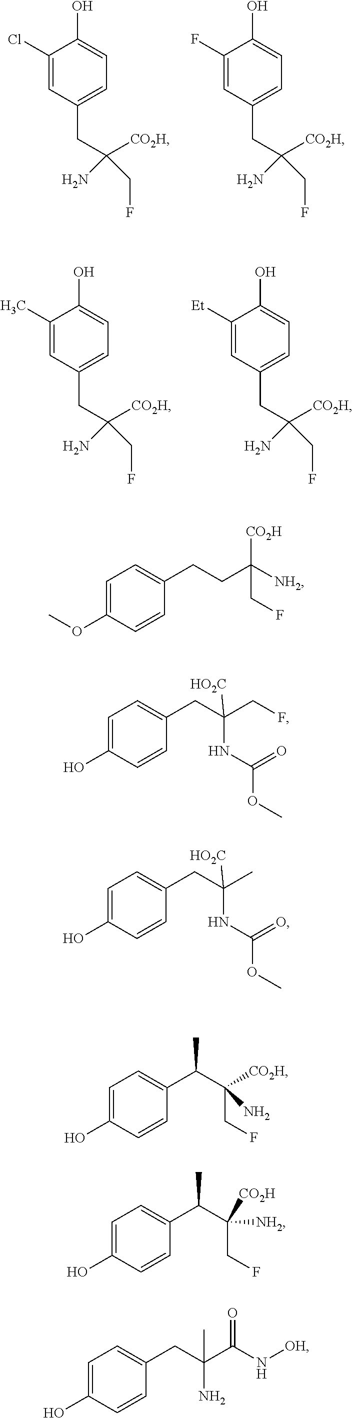

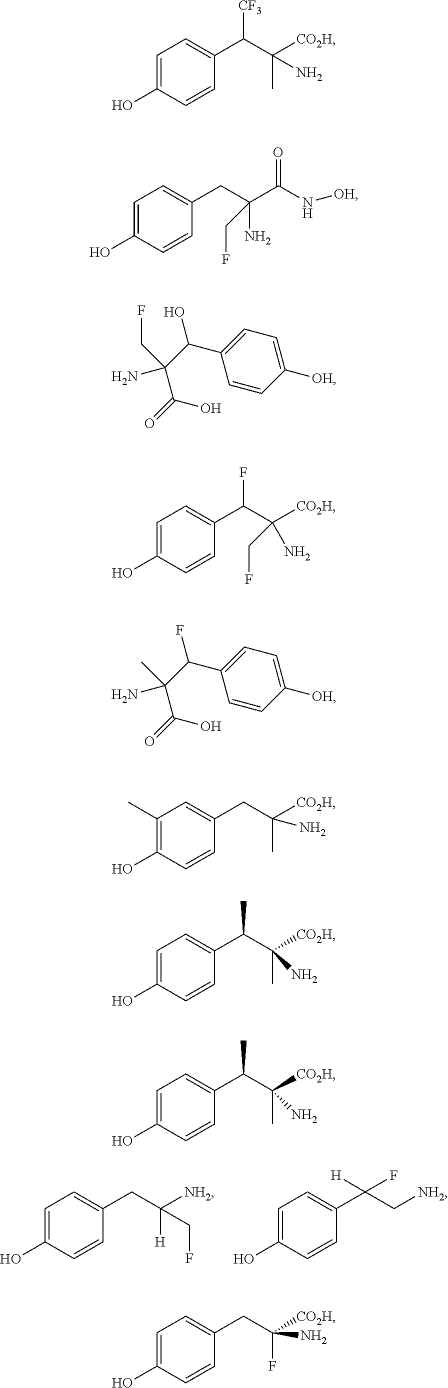

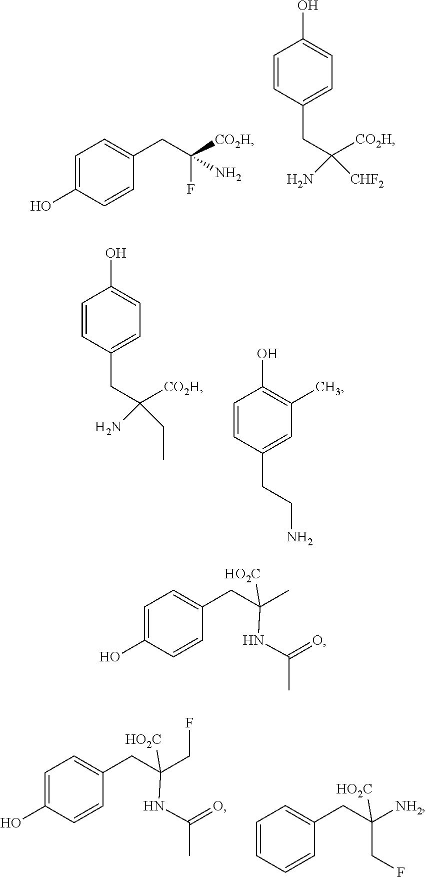

and pharmaceutically acceptable salts thereof.

In some embodiments, the tyrosine decarboxylase inhibitor is formulated as a pharmaceutical composition comprising a pharmaceutically acceptable excipient and at least one compound chosen from compounds of formulas (I) (I-1), (II), compounds of the previously described groups above, and pharmaceutically acceptable salts thereof.

The term “acyl,” as used herein, represents a chemical substituent of formula —C(O)—R, wherein R is alkyl, alkenyl, aryl, arylalkyl, cycloalkyl, heterocyclyl, heterocyclyl alkyl, heteroaryl, or heteroaryl alkyl. An optionally substituted acyl is an acyl that is optionally substituted as described herein for each group R. Non-limiting examples of acyl include fatty acid acyls (e.g., short chain fatty acid acyls (e.g., acetyl, propionyl, or butyryl)).

The term “acylated sugar,” as used herein, refers to a carbohydrate, sugar acid, or sugar alcohol having one or more hydroxyls substituted with an acyl (e.g., a fatty acid acyl). In some embodiments, the carbohydrate is a monosaccharide. In some embodiments, the fatty acid acyl is a short chain fatty acid acyl (e.g., propionyl or butyryl). An acylated sugar can be a compound or a monovalent group. When an acylated sugar is a monovalent group, the group includes one and only one valency for attaching to another molecular fragment. When an acylated sugar is covalently bonded to a carbon atom of another molecular fragment, the valency is on an oxygen atom of the acylated sugar. When an acylated sugar is covalently bonded to an oxygen atom of another molecular fragment, the valency is on the anomeric carbon atom of the acylated sugar. Non-limiting examples of monosaccharides include arabinose, xylose, fructose, galactose, glucose, glucosinolate, ribose, tagatose, fucose, and rhamnose. Non-limiting examples of sugar acids include xylonic acid, gluconic acid, glucuronic acid, galacturonic acid, tartaric acid, saccharic acid, and mucic acid. Non-limiting examples of sugar alcohols include glycerol, erythritol, theritol, arabitol, xylitol, tibitol, mannitol, sorbitol, galactitol, fucitol, iditol, and inositol.

The term “acyloxy,” as used herein, represents a chemical substituent of formula —OR, wherein R is acyl. An optionally substituted acyloxy is an acyloxy that is optionally substituted as described herein for acyl.

The term “alcohol oxygen atom,” as used herein, refers to a divalent oxygen atom, wherein at least one valency of the oxygen atom is bonded to an spa-hybridized carbon atom.

The term “alkanoyl,” as used herein, represents a chemical substituent of formula —C(O)—R, wherein R is alkyl. An optionally substituted alkanoyl is an alkanoyl that is optionally substituted as described herein for alkyl.

The term “alkoxy,” as used herein, represents a chemical substituent of formula —OR, wherein R is a C₁₋₆ alkyl group, unless otherwise specified. An optionally substituted alkoxy is an alkoxy group that is optionally substituted as defined herein for alkyl.

The term “alkenyl,” as used herein, represents acyclic monovalent straight or branched chain hydrocarbon groups containing one, two, or three carbon-carbon double bonds. Alkenyl, when unsubstituted, has from 2 to 12 carbon atoms (e.g., 1 to 8 carbons), unless specified otherwise. Non-limiting examples of alkenyl groups include ethenyl, prop-1-enyl, prop-2-enyl, 1-methylethenyl, but-1-enyl, but-2-enyl, but-3-enyl, 1-methylprop-1-enyl, 2-methylprop-1-enyl, and 1-methylprop-2-enyl. Alkenyl groups may be optionally substituted as defined herein for alkyl.

The term “alkenylene,” as used herein, refers to a divalent, straight or branched, unsaturated hydrocarbon including one, two, or three carbon-carbon double bonds, in which two valencies replace two hydrogen atoms. Alkenylene, when unsubstituted, has from 2 to 12 carbon atoms (e.g., 2 to 6 carbons), unless specified otherwise. Non-limiting examples of alkenylene groups include ethen-1,1-diyl; ethen-1,2-diyl; prop-1-en-1,1-diyl, prop-2-en-1,1-diyl; prop-1-en-1,2-diyl, prop-1-en-1,3-diyl; prop-2-en-1,1-diyl; prop-2-en-1,2-diyl; but-1-en-1,1-diyl; but-1-en-1,2-diyl; but-1-en-1,3-diyl; but-1-en-1,4-diyl; but-2-en-1,1-diyl; but-2-en-1,2-diyl; but-2-en-1,3-diyl; but-2-en-1,4-diyl; but-2-en-2,3-diyl; but-3-en-1,1-diyl; but-3-en-1,2-diyl; but-3-en-1,3-diyl; but-3-en-2,3-diyl; buta-1,2-dien-1,1-diyl; buta-1,2-dien-1,3-diyl; buta-1,2-dien-1,4-diyl; buta-1,3-dien-1,1-diyl; buta-1,3-dien-1,2-diyl; buta-1,3-dien-1,3-diyl; buta-1,3-dien-1,4-diyl; buta-1,3-dien-2,3-diyl; buta-2,3-dien-1,1-diyl; and buta-2,3-dien-1,2-diyl. An optionally substituted alkenylene is an alkenylene that is optionally substituted as described herein for alkyl.

The term “alkyl,” as used herein, refers to an acyclic, straight or branched, saturated hydrocarbon group, which, when unsubstituted, has from 1 to 12 carbons (e.g., 1 to 6 carbons), unless otherwise specified. Alkyl groups are exemplified by methyl; ethyl; n- and iso-propyl; n-, sec-, iso- and tert-butyl; neopentyl, and the like, and may be optionally substituted, valency permitting, with one, two, three, or, in the case of alkyl groups of two carbons or more, four or more substituents independently selected from: alkoxy; acyloxy; alkylsulfinyl; alkylsulfonyl; amino; aryl; aryloxy; azido; cycloalkyl; cycloalkoxy; halo; heterocyclyl; heteroaryl; heterocyclylalkyl; heteroarylalkyl; heterocyclyloxy; heteroaryloxy; hydroxy; nitro; thioalkyl; thioalkenyl; thioaryl; thiol; cyano; oxo (═O); thio (═S); and imino (═NR′), wherein R′ is H, alkyl, aryl, or heterocyclyl. Each of the substituents may itself be unsubstituted or, valency permitting, substituted with unsubstituted substituent(s) defined herein for each respective group.

The term “alkylene,” as used herein, refers to a divalent, straight or branched, saturated hydrocarbon, in which two valencies replace two hydrogen atoms. Alkyl, when unsubstituted, has from 2 to 12 carbon atoms (e.g., 2 to 6 carbons), unless specified otherwise. Non-limiting examples of alkylene groups include methylene, ethane-1,2-diyl, ethane-1,1-diyl, propane-1,3-diyl, propane-1,2-diyl, propane-1,1-diyl, propane-2,2-diyl, butane-1,4-diyl, butane-1,3-diyl, butane-1,2-diyl, butane-1,1-diyl, and butane-2,2-diyl, butane-2,3-diyl. An optionally substituted alkylene is an alkylene that is optionally substituted as described herein for alkyl.

The term “alkylsulfinyl,” as used herein, represents a group of formula —S(O)-(alkyl). An optionally substituted alkylsulfinyl is an alkylsulfinyl that is optionally substituted as described herein for alkyl.

The term “alkylsulfonyl,” as used herein, represents a group of formula —S(O)₂-(alkyl). An optionally substituted alkylsulfonyl is an alkylsulfonyl that is optionally substituted as described herein for alkyl.

The term “alkynyl,” as used herein, represents an acyclic, monovalent, straight or branched chain hydrocarbon groups containing one, two, or three carbon-carbon triple bonds. Alkynyl, when unsubstituted, has from 2 to 12 carbon atoms (e.g., 2 to 6 carbons), unless specified otherwise. Non-limiting examples of alkynyl groups include ethynyl, prop-1-ynyl, prop-2-ynyl, but-1-ynyl, but-2-ynyl, but-3-ynyl, and 1-methylprop-2-ynyl. An optionally substituted alkynyl is an alkynyl that is optionally substituted as defined herein for alkyl.

The term “alkynylene,” as used herein, refers to a divalent, straight, or branched, unsaturated hydrocarbon including one, two, or three carbon-carbon triple bonds, in which two valencies replace two hydrogen atoms. Alkynylene, when unsubstituted, has from 2 to 12 carbon atoms (e.g., 2 to 6 carbons), unless specified otherwise. Non-limiting examples of alkynylene groups include ethyn-1,2-diyl; prop-1-yn-1,3-diyl; prop-2-yn-1,1-diyl; but-1-yn-1,3-diyl; but-1-yn-1,4-diyl; but-2-yn-1,1-diyl; but-2-yn-1,4-diyl; but-3-yn-1,1-diyl; but-3-yn-1,2-diyl; but-3-yn-2,2-diyl; and buta-1,3-diyn-1,4-diyl. An optionally substituted alkynylene is an alkynylene that is optionally substituted as described herein for alkyl.

The term “aryl,” as used herein, represents a mono-, bicyclic, or multicyclic carbocyclic ring system having one or two aromatic rings. Aryl group may include from 6 to 10 carbon atoms. All atoms within an unsubstituted carbocyclic aryl group are carbon atoms. Non-limiting examples of carbocyclic aryl groups include phenyl, naphthyl, 1,2-dihydronaphthyl, 1,2,3,4-tetrahydronaphthyl, fluorenyl, indanyl, indenyl, etc. The aryl group may be unsubstituted or substituted with one, two, three, four, or five substituents independently selected from: alkyl; alkenyl; alkoxy; acyloxy; amino; aryl; aryloxy; azido; cycloalkyl; cycloalkoxy; halo; heterocyclyl; heteroaryl; heterocyclylalkyl; heteroarylalkyl; heterocyclyloxy; heteroaryloxy; hydroxy; nitro; thioalkyl; thioalkenyl; thioaryl; thiol; and cyano. Each of the substituents may itself be unsubstituted or substituted with unsubstituted substituent(s) defined herein for each respective group.

The term “aryl alkyl,” as used herein, represents an alkyl group substituted with an aryl group. An optionally substituted aryl alkyl is an aryl alkyl, in which aryl and alkyl portions may be optionally substituted as the individual groups as described herein.

The term “arylene,” as used herein, is a divalent group that is an aryl group, in which one hydrogen atom is replaced with a valency. Arylene may be optionally substituted as described herein for aryl. Non-limiting examples of arylenes include phenylene (e.g., 1,2-phenylene, 1,3-phenylene, and 1.4-phenylene).

The term “aryloxy,” as used herein, represents a group —OR, wherein R is aryl. Aryloxy may be an optionally substituted aryloxy. An optionally substituted aryloxy is aryloxy that is optionally substituted as described herein for aryl.

The term “carbamate linker,” as used herein, refers to a group R¹—(CO)—R², wherein R¹ is a bond to an alcohol or phenolic oxygen atom, and R² is a bond to a nitrogen atom.

The term “carbohydrate,” as used herein, refers to a monosaccharide, disaccharide, or an oligosaccharide or an analog of the following structure:

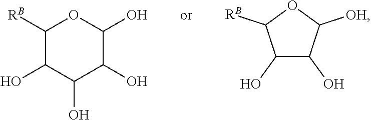

wherein R^{B} is H, optionally substituted C₁₋₆ alkyl, or —CH₂—OH.

The term “carbohydrate” may refer to a compound or to a monovalent or multivalent chemical substituent. When the term “carbohydrate” refers to a chemical substituent, the valence(s) reside on the anomeric carbon atom and/or alcohol oxygen atoms. An optionally substituted carbohydrate is a carbohydrate, in which at least one hydroxyl is substituted with an acyl (e.g., a fatty acid acyl).

The term “carbonate linker,” as used herein, refers to a group R¹—C(O)—R², wherein R¹ is a bond to a first alcohol or phenolic oxygen atom, and R² is a bond to a second alcohol or phenolic oxygen atom.

The term “carbonyl,” as used herein, refers to a divalent group —C(O)—.

The term “carboxylate,” as used herein, represents group —COOH or a salt thereof.

The term “cycloalkylene,” as used herein, represents a divalent group that is a cycloalkyl group, in which one hydrogen atom is replaced with a valency. An optionally substituted cycloalkylene is a cycloalkylene that is optionally substituted as described herein for cycloalkyl.

The term “cycloalkoxy,” as used herein, represents a group —OR, wherein R is cycloalkyl. An optionally substituted cycloalkoxy is cycloalkoxy that is optionally substituted as described herein for cycloalkyl.

The term “dialkylamino,” as used herein, refers to a group —NR₂, wherein each R is independently alkyl.

The term “ester bond,” as used herein, refers to a covalent bond between an alcohol or phenolic oxygen atom and a carbonyl group that is further bonded to a carbon atom.

The term “fatty acid,” as used herein, refers to a short-chain fatty acid, a medium chain fatty acid, a long chain fatty acid, a very long chain fatty acid, or an unsaturated analogue thereof, or a phenyl-substituted analogue thereof. Short chain fatty acids contain from 1 to 6 carbon atoms, medium chain fatty acids contain from 7 to 13 carbon atoms, and a long-chain fatty acids contain from 14 to 22 carbon atoms. A fatty acid may be saturated or unsaturated. An unsaturated fatty acid includes 1, 2, 3, 4, 5, or 6 carbon-carbon double bonds. In some embodiments, the carbon-carbon double bonds in unsaturated fatty acids have Z stereochemistry.

The term “fatty acid acyl,” as used herein, refers to a fatty acid, in which the hydroxyl group is replaced with a valency. In some embodiments, a fatty acid acyl is a short chain fatty acid acyl.

The term “fatty acid acyloxy,” as used herein, refers to group —OR, wherein R is a fatty acid acyl.

The term “fluoroalkyl,” as used herein, refers to a C₁₋₆ alkyl group that is substituted with one or more fluorine atoms; the number of fluorine atoms is up to the total number of hydrogen atoms available for replacement with fluorine atoms. A fluoroalkyl in which all hydrogen atoms were replaced with fluorine atoms is a perfluoroalkyl. Non-limiting examples of perfluoroalkyls include trifluoromethyl and pentafluoroethyl.

The term “glycoside,” as used herein, refers to a monovalent group that is a monosaccharide or sugar acid having a valency on an anomeric carbon. Non-limiting examples of monosaccharides include arabinose, xylose, fructose, galactose, glucose, ribose, tagatose, fucose, and rhamnose. Non-limiting examples of sugar acids include xylonic acid, gluconic acid, glucuronic acid, galacturonic acid, tartaric acid, saccharic acid, and mucic acid.

The term “glycosidic bond,” as used herein, refers to a covalent bond between an oxygen atom and an anomeric carbon atom in a monosaccharide or sugar acid having an anomeric carbon atom.

The term “halogen,” as used herein, represents a halogen selected from bromine, chlorine, iodine, and fluorine.

The term “heteroaryl,” as used herein, represents a monocyclic 5-, 6-, 7-, or 8-membered ring system, or a fused or bridging bicyclic, tricyclic, or tetracyclic ring system; the ring system contains one, two, three, or four heteroatoms independently selected from nitrogen, oxygen, and sulfur; and at least one of the rings is an aromatic ring. Non-limiting examples of heteroaryl groups include benzimidazolyl, benzofuryl, benzothiazolyl, benzothienyl, benzoxazolyl, furyl, imidazolyl, indolyl, isoindazolyl, isoquinolinyl, isothiazolyl, isothiazolyl, isoxazolyl, oxadiazolyl, oxazolyl, purinyl, pyrrolyl, pyridinyl, pyrazinyl, pyrimidinyl, qunazolinyl, quinolinyl, thiadiazolyl (e.g., 1,3,4-thiadiazole), thiazolyl, thienyl, triazolyl, tetrazolyl, dihydroindolyl, tetrahydroquinolyl, tetrahydroisoquinolyl, etc. The term bicyclic, tricyclic, and tetracyclic heteroaryls include at least one ring having at least one heteroatom as described above and at least one aromatic ring. For example, a ring having at least one heteroatom may be fused to one, two, or three carbocyclic rings, e.g., an aryl ring, a cyclohexane ring, a cyclohexene ring, a cyclopentane ring, a cyclopentene ring, or another monocyclic heterocyclic ring. Examples of fused heteroaryls include 1,2,3,5,8,8a-hexahydroindolizine; 2,3-dihydrobenzofuran; 2,3-dihydroindole; and 2,3-dihydrobenzothiophene. Heteroaryl may be optionally substituted with one, two, three, four, or five substituents independently selected from: alkyl; alkenyl; alkoxy; acyloxy; aryloxy; alkylsulfinyl; alkylsulfonyl; amino; arylalkoxy; cycloalkyl; cycloalkoxy; halogen; heterocyclyl; heterocyclyl alkyl; heteroaryl; heteroaryl alkyl; heterocyclyloxy; heteroaryloxy; hydroxyl; nitro; thioalkyl; thioalkenyl; thioaryl; thiol; cyano; ═O; —NR₂, wherein each R is independently hydrogen, alkyl, acyl, aryl, arylalkyl, cycloalkyl, heterocyclyl, or heteroaryl; —COOR^{A}, wherein R^{A} is hydrogen, alkyl, aryl, arylalkyl, cycloalkyl, heterocyclyl, or heteroaryl; and —CON(R^{B})₂, wherein each R^{B} is independently hydrogen, alkyl, aryl, arylalkyl, cycloalkyl, heterocyclyl, or heteroaryl. Each of the substituents may itself be unsubstituted or substituted with unsubstituted substituent(s) defined herein for each respective group.

The term “heteroarylene,” as used herein, is a divalent group that is a heteroaryl group, in which one hydrogen atom is replaced with a valency. Heteroarylene may be optionally substituted as described herein for heteroaryl.

The term “heteroaryloxy,” as used herein, refers to a structure —OR, in which R is heteroaryl. Heteroaryloxy can be optionally substituted as defined for heteroaryl.

The term “heterocyclyl,” as used herein, represents a monocyclic, bicyclic, tricyclic, or tetracyclic non-aromatic ring system having fused or bridging 4-, 5-, 6-, 7-, or 8-membered rings, unless otherwise specified, the ring system containing one, two, three, or four heteroatoms independently selected from nitrogen, oxygen, and sulfur. Non-aromatic 5-membered heterocyclyl has zero or one double bonds, non-aromatic 6- and 7-membered heterocyclyl groups have zero to two double bonds, and non-aromatic 8-membered heterocyclyl groups have zero to two double bonds and/or zero or one carbon-carbon triple bond. Heterocyclyl groups have a carbon count of 1 to 16 carbon atoms unless otherwise specified. Certain heterocyclyl groups may have a carbon count up to 9 carbon atoms. Non-aromatic heterocyclyl groups include pyrrolinyl, pyrrolidinyl, pyrazolinyl, pyrazolidinyl, imidazolinyl, imidazolidinyl, piperidinyl, homopiperidinyl, piperazinyl, pyridazinyl, oxazolidinyl, isoxazolidiniyl, morpholinyl, thiomorpholinyl, thiazolidinyl, isothiazolidinyl, thiazolidinyl, tetrahydrofuranyl, dihydrofuranyl, tetrahydrothienyl, dihydrothienyl, pyranyl, dihydropyranyl, dithiazolyl, etc. The term “heterocyclyl” also represents a heterocyclic compound having a bridged multicyclic structure in which one or more carbons and/or heteroatoms bridges two non-adjacent members of a monocyclic ring, e.g., quinuclidine, tropanes, or diaza-bicyclo[2.2.2]octane. The term “heterocyclyl” includes bicyclic, tricyclic, and tetracyclic groups in which any of the above heterocyclic rings is fused to one, two, or three carbocyclic rings, e.g., a cyclohexane ring, a cyclohexene ring, a cyclopentane ring, a cyclopentene ring, or another heterocyclic ring. Examples of fused heterocyclyls include 1,2,3,5,8,8a-hexahydroindolizine; 2,3-dihydrobenzofuran; 2,3-dihydroindole; and 2,3-dihydrobenzothiophene. The heterocyclyl group may be unsubstituted or substituted with one, two, three, four or five substituents independently selected from: alkyl; alkenyl; alkoxy; acyloxy; alkylsulfinyl; alkylsulfonyl; aryloxy; amino; arylalkoxy; cycloalkyl; cycloalkoxy; halogen; heterocyclyl; heterocyclyl alkyl; heteroaryl; heteroaryl alkyl; heterocyclyloxy; heteroaryloxy; hydroxyl; nitro; thioalkyl; thioalkenyl; thioaryl; thiol; cyano; ═O; ═S; —NR₂, wherein each R is independently hydrogen, alkyl, acyl, aryl, arylalkyl, cycloalkyl, heterocyclyl, or heteroaryl; —COOR^{A}, wherein R^{A} is hydrogen, alkyl, aryl, arylalkyl, cycloalkyl, heterocyclyl, or heteroaryl; and —CON(R^{B})₂, wherein each R^{B} is independently hydrogen, alkyl, aryl, arylalkyl, cycloalkyl, heterocyclyl, or heteroaryl.

The term “heterocyclyl alkyl,” as used herein, represents an alkyl group substituted with a heterocyclyl group. The heterocyclyl and alkyl portions of an optionally substituted heterocyclyl alkyl are optionally substituted as the described for heterocyclyl and alkyl, respectively.

The term “heterocyclylene,” as used herein, represents a heterocyclyl, in which one hydrogen atom is replaced with a valency. An optionally substituted heterocyclylene is a heterocyclylene that is optionally substituted as described herein for heterocyclyl.

The term “heterocyclyloxy,” as used herein, refers to a structure —OR, in which R is heterocyclyl. Heterocyclyloxy can be optionally substituted as described for heterocyclyl.

The terms “hydroxyl” and “hydroxy,” as used interchangeably herein, represent —OH. A hydroxyl substituted with an acyl is an acyloxy. A protected hydroxyl is a hydroxyl, in which the hydrogen atom is replaced with an O-protecting group.

The term “hydroxyalkyl,” as used herein, refers to a C1-6 alkyl group that is substituted with one or more hydroxyls, provided that each carbon atom in the hydroxyalkyl is attached either to no more than one hydroxyl. Non-limiting examples of hydroxyalkyls include hydroxymethyl, 2-hydroxyethyl, and 1-hydroxyethyl.

The term “hydroxycinnamic acid,” as used herein, refers to a cinnamic acid having one, two, or three hydroxyls attached to the phenyl ring of the hydroxycinnamic acid. A non-limiting example of a hydroxycinnamic acid is caffeic acid.

The term “oxo,” as used herein, represents a divalent oxygen atom (e.g., the structure of oxo may be shown as ═O).

The term “pharmaceutically acceptable salt,” as used herein, represents those salts which are, within the scope of sound medical judgment, suitable for use in contact with the tissues of humans and animals without undue toxicity, irritation, allergic response and the like and are commensurate with a reasonable benefit/risk ratio. Principles for preparing pharmaceutically acceptable salts are well known in the art. For example, pharmaceutically acceptable salts are described in: Berge et al., J. Pharmaceutical Sciences 1977; 66:1-19, and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting the free base group with a suitable electrophile. Representative counterions useful for pharmaceutically acceptable salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, bromide, chloride, iodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like.

The term “phenolic oxygen atom,” as used herein, refers to a divalent oxygen atom within the structure of a compound, wherein at least one valency of the phenolic oxygen atom is bonded to an sp2-hybridized carbon atom within an aromatic ring.

The term “protecting group,” as used herein, represents a group intended to protect a hydroxy, an amino, or a carbonyl from participating in one or more undesirable reactions during chemical synthesis. The term “0-protecting group,” as used herein, represents a group intended to protect a hydroxy or carbonyl group from participating in one or more undesirable reactions during chemical synthesis. The term “N-protecting group,” as used herein, represents a group intended to protect a nitrogen containing (e.g., an amino or hydrazine) group from participating in one or more undesirable reactions during chemical synthesis. Commonly used O- and N-protecting groups are disclosed in Greene, “Protective Groups in Organic Synthesis,” 3^{rd} Edition (John Wiley & Sons, New York, 1999), which is incorporated herein by reference. Exemplary 0- and N-protecting groups include alkanoyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, t-butyldimethylsilyl, tri-iso-propylsilyloxymethyl, 4,4′-dimethoxytrityl, isobutyryl, phenoxyacetyl, 4-isopropyl pehenoxyacetyl, dimethylformamidino, and 4-nitrobenzoyl.

Exemplary O-protecting groups for protecting carbonyl containing groups include, but are not limited to: acetals, acylals, 1,3-dithianes, 1,3-dioxanes, 1,3-dioxolanes, and 1,3-dithiolanes.

Other O-protecting groups include, but are not limited to: substituted alkyl, aryl, and aryl-alkyl ethers (e.g., trityl; methylthiomethyl; methoxymethyl; benzyloxymethyl; siloxymethyl; 2,2,2,-trichloroethoxymethyl; tetrahydropyranyl; tetrahydrofuranyl; ethoxyethyl; 1-[2-(trimethylsilyl)ethoxy]ethyl; 2-trimethylsilylethyl; t-butyl ether; p-chlorophenyl, p-methoxyphenyl, p-nitrophenyl, benzyl, p-methoxybenzyl, and nitrobenzyl); silyl ethers (e.g., trimethylsilyl; triethylsilyl; triisopropylsilyl; dimethylisopropylsilyl; t-butyldimethylsilyl; t-butyldiphenylsilyl; tribenzylsilyl; triphenylsilyl; and diphenymethylsilyl); carbonates (e.g., methyl, methoxymethyl, 9-fluorenylmethyl; ethyl; 2,2,2-trichloroethyl; 2-(trimethylsilyl)ethyl; vinyl, allyl, nitrophenyl; benzyl; methoxybenzyl; 3,4-dimethoxybenzyl; and nitrobenzyl).

Other N-protecting groups include, but are not limited to, chiral auxiliaries such as protected or unprotected D, L or D, L-amino acids such as alanine, leucine, phenylalanine, and the like; sulfonyl-containing groups such as benzenesulfonyl, p-toluenesulfonyl, and the like; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyl oxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, phenylthiocarbonyl, and the like, aryl-alkyl groups such as benzyl, triphenylmethyl, benzyloxymethyl, and the like and silyl groups such as trimethylsilyl, and the like. Useful N-protecting groups are formyl, acetyl, benzoyl, pivaloyl, t-butylacetyl, alanyl, phenylsulfonyl, benzyl, t-butyloxycarbonyl (Boc), and benzyloxycarbonyl (Cbz).

The term “sugar acid,” as used herein, refers to a monosaccharide, in the linear form of which, one or both terminal positions are oxidized to a carboxylic acid. There are four classes of sugar acids: aldonic acid, ulosonic acid, uronic acid, and aldaric acid. Any of the four sugar acid classes may be used in compounds disclosed herein. Non-limiting examples of sugar acids include xylonic acid, gluconic acid, glucuronic acid, galacturonic acid, tartaric acid, saccharic acid, and mucic acid.

The term “sugar acid acyl,” as used herein, refers to a monovalent group that is a sugar acid having a carboxylate, in which —OH is replaced with a valency.

The term “thioalkenyl,” as used herein, represents a group —SR, wherein R is alkenyl. An optionally substituted thioalkenyl is thioalkenyl that is optionally substituted as described herein for alkenyl.

The term “thioalkyl,” as used herein, represents a group —SR, wherein R is alkyl. An optionally substituted thioalkyl is thioalkyl that is optionally substituted as described herein for alkyl.

The term “thioaryl,” as used herein, represents a group —SR, wherein R is aryl. An optionally substituted thioaryl is thioaryl that is optionally substituted as described herein for aryl.

The compounds described herein, unless otherwise noted, encompass isotopically enriched compounds (e.g., deuterated compounds), tautomers, and all stereoisomers and conformers (e.g., enantiomers, diastereomers, E/Z isomers, atropisomers, etc.), as well as racemates thereof and mixtures of different proportions of enantiomers or diastereomers, or mixtures of any of the foregoing forms as well as salts (e.g., pharmaceutically acceptable salts).

In some embodiments, the compounds described herein may be a conjugate, e.g., compounds including a glycoside or an acylated sugar. In some embodiments, the compound is a conjugate comprising at least one glycoside or acylated sugar. In some embodiments, upon administration of the conjugate, the conjugate may be cleaved in vivo to remove the glycoside or an acylated sugar from the compound and to release the corresponding unconjugated compound. In some embodiments, conjugates may be advantageous in therapeutic applications benefitting from a particular tissue-targeted delivery of an unconjugated compound.

In some embodiments, the compounds described herein that include at least one glycoside or at least one acylated sugar are conjugates. In some embodiments, compounds having a fatty acid acyl (e.g., a short chain fatty acid acyl) attached through an ester bond are also conjugates.

Acylated sugars that may be used in the conjugates described herein include an acyl (e.g., a fatty acid acyl) and a core selected from a carbohydrate (e.g., a monosaccharide), sugar acid, and sugar alcohol. For example, an acylated sugar may be a monovalent group of formula (III):

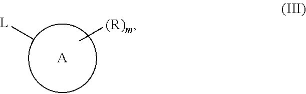

wherein

L is a bond to a pharmaceutically active agent, a carbonate linker, or a carbamate linker;

group A is a core selected from a carbohydrate (e.g., a monosaccharide), sugar acid, and sugar alcohol;

each R is independently an acyl bonded to an oxygen atom in group A; and

m is an integer from 0 to the total number of available hydroxyl groups in group A (e.g., 1, 2, 3, 4, or 5).

In some embodiments of formula (III), L may be attached to a carbon atom in group A (e.g., an anomeric carbon atom or a carbonyl carbon atom). In some embodiments, L may be attached to an oxygen atom in group A (e.g., an alcoholic oxygen atom, a phenolic oxygen atom, or a carboxylate oxygen atom).

In some embodiments of formula (III), at least one R is a fatty acid acyl.

In some embodiments of formula (III), the fatty acid(s) are short chain fatty acid acyls. In some embodiments, the short chain fatty acid acyl is a 03-6 short chain fatty acid acyl (e.g., propionyl or butyryl).

In some embodiments of formula (III), the acylated sugar is peracylated, i.e., all of the available hydroxyls in the acylated sugar are substituted with an acyl.

A monosaccharide may be, e.g., arabinose, xylose, fructose, galactose, glucose, ribose, tagatose, fucose, or rhamnose. In some embodiments, the monosaccharide is L-arabinose, D-xylose, fructose, galactose, D-glucose, D-ribose, D-tagatose, L-fucose, or L-rhamnose (e.g., the monosaccharide is D-xylose). A sugar acid may be, e.g., aldonic acid, ulosonic acid, uronic acid, or aldaric acid. A sugar acid may be, e.g., xylonic acid, gluconic acid, glucuronic acid, galacturonic acid, tartaric acid, saccharic acid, or mucic acid. A sugar alcohol may be, e.g., glycerol, erythritol, threitol, arabitol, xylitol, tibitol, mannitol, sorbitol, galactitol, fucitol, iditol, or inositol.

An acylated sugar may be covalently linked to a pharmaceutically active agent through a carbon-oxygen bond that is cleavable in vivo, a carbonate linker, or a carbamate linker. The carbon-oxygen bond may be, e.g., a glycosidic bond or ester bond. Acylated sugars having a monosaccharide or a sugar acid as a core may be covalently linked to a pharmaceutically active agent through a carbon-oxygen bond that is cleavable in vivo (e.g., a glycosidic bond or ester bond), a carbonate linker, or a carbamate linker. In the sugar acid core, one or both carboxylates may be present as O-protected versions (e.g., as alkyl esters (e.g., methyl or ethyl esters)). Acylated sugars having a sugar alcohol as a core may be covalently linked to a pharmaceutically active agent through a carbon-oxygen bond that is cleavable in vivo (e.g., an ester bond), a carbonate linker, or a carbamate linker.

Non-limiting examples of acylated sugars are:

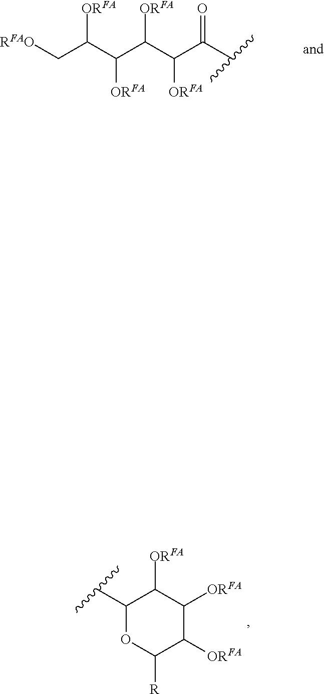

wherein

R is H, —CH₃, or —CH₂OR^{FA}; and

each R^{FA} is independently H or a fatty acid acyl (e.g., a short chain fatty acid acyl);

provided that at least one R^{FA} is a fatty acid acyl (e.g., a short chain fatty acid acyl).

In some embodiments, the tyrosine decarboxylase inhibitor is a compound of formula (I):

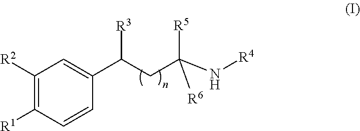

or a pharmaceutically acceptable salt thereof,
wherein

n is 0 or 1;

R¹ is H or —OR^{A}, wherein R^{A} is H, —C(O)C₁₋₆ alkyl, or an acylated sugar;

R² is H, halogen, amino, C₁₋₆ alkyl, or —OR^{A}, wherein R^{A} is H or an acylated sugar;

R³ is H, a halogen, —OH, or C₁₋₆ alkyl optionally substituted with one or more halogens;

R⁴ is H, —NH₂, —C(O)OCH₃, or an acylated sugar;

R⁵ is H, —C(O)OH, —C(O)OC₁₋₆ alkyl, —C(O)Oglycoside, —C(O)NHOH, or —C(O)O(acylated sugar); and

R⁶ is H, halogen, or optionally substituted C₁₋₆ alkyl;

provided that at least one R^{A} is present; or provided that R³ and/or R⁶ comprise a halogen.

In some embodiments, the compound of formula (I) is a compound of formula (I-a):

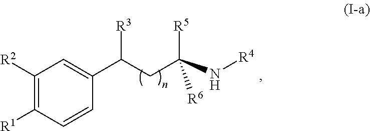

or a pharmaceutically acceptable salt thereof.

In some embodiments, the compound of formula (I) is a compound of formula (II):

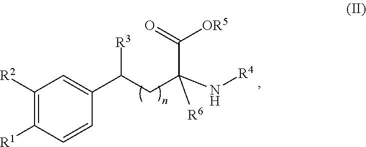

or a pharmaceutically acceptable salt thereof,
wherein

n is 0 or 1;

each of R¹ and R² is independently H or —OR^{A}, wherein each R^{A} is independently H or an acylated sugar, or R¹ is —C(O)C₁₋₆ alkyl;

R³ is H or a halogen;

R⁴ is H, —NH₂, —C(O)OCH₃, or an acylated sugar;

R⁵ is H, alkyl, glycoside, or an acylated sugar; and

R⁶ is H or optionally substituted alkyl;

provided that at least one R^{A} is present; or provided that R³ and/or R⁶ comprise a halogen.

In some embodiments, the compound is a compound of formula (II-a):

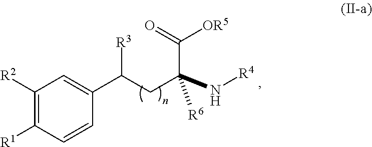

or a pharmaceutically acceptable salt thereof.

In some embodiments of formula (II-a), R is H. In some embodiments, R is methyl.

In some embodiments of formula (II-a), R¹ is H or —OH. In some embodiments, R¹ is H. In some embodiments, R¹ is —OH. In some embodiments, R¹ is —OC(O)C₁₋₆ alkyl. In some embodiments, R¹ is —OC(O)CH₃. In some embodiments, R¹ is —OC(O)CH₂CH₃. In some embodiments, R¹ is —OC(O)CH₂CH₂CH₃. In some embodiments, R¹ is —O(acylated sugar).

In some embodiments of formula (II-a), R¹ is —OH and R² is H. In some embodiments, R¹ is —OH and R² is H.

In some embodiments of formula (II-a), R¹ is —OH and R² is H. In some embodiments, R¹ is —OH R² is a halogen.

In some embodiments of formula (II-a), R² is an amino. In some embodiments, R² is C₁₋₆ alkyl. In some embodiments, R² is methyl.

In some embodiments of formula (II-a), R³ is H. In some embodiments, R³ is a halogen. In some embodiments, R³ is fluoro or chloro. In some embodiments, R³ is OH. In some embodiments, R³ is a C₁₋₆ alkyl optionally substituted with one or more halogens. In some embodiments, R³ is methylene optionally substituted with one or more halogens. In some embodiments, R³ is methyl.

In some embodiments of formula (II-a), R⁴ is H. In some embodiments, R⁴ is —NH₂.

In some embodiments of formula (II-a), R⁵ is —C(O)OH. In some embodiments, R⁵ is —C(O)Oacylated sugar. In some embodiments, R⁵ is H. In some embodiments, R⁵ is —C(O)OC₁₋₆alkyl. In some embodiments, R⁵ is —C(O)OCH₃. In some embodiments, R⁵ is C(O)Oglycoside. In some embodiments, R⁵ is C(O)NHOH.

In some embodiments of formula (II-a), R⁶ is H. In some embodiments, R⁶ is a C₁₋₆ alkyl. In some embodiments, R⁶ is a C₁₋₆ alkyl substituted with one, two, or three halogens. In some embodiments, R⁶ is a C₁₋₆ alkyl substituted with one, two, or three fluorine atoms. In some embodiments, R⁶ is a halogen. In some embodiments, R⁶ is methyl. In some embodiments, R⁶ is ethyl.

In some embodiments of formula (II-a), n is 0. In some embodiments, n is 1.

In some embodiments, the tyrosine decarboxylase inhibitor is chosen from compounds of formula (I) and pharmaceutically acceptable salts thereof, wherein

n is 0;

R¹ is —OH;

R² is halogen;

R³ is H, a halogen, or —OH, C₁₋₆ alkyl optionally substituted with one or more halogens;

R⁴ is H, —NH₂, or an acylated sugar;

R⁵ is H, —C(O)OH, —C(O)OC₁₋₆ alkyl, —C(O)Oglycoside, —C(O)NHOH, or —C(O)O(acylated sugar); and

R⁶ is H or optionally substituted C₁₋₆ alkyl.

In some embodiments, the tyrosine decarboxylase inhibitor is chosen from compounds of formula (I) and pharmaceutically acceptable salts thereof, wherein

n is 0;

R¹ is —OH;

R² is halogen;

R³ is H;

R⁴ is H;

R⁵ is —C(O)OH; and

R⁶ is optionally substituted alkyl. In some embodiments, R⁶ is methylene substituted with one or more halogens or hydroxy.

In some embodiments, the tyrosine decarboxylase inhibitor is chosen from:

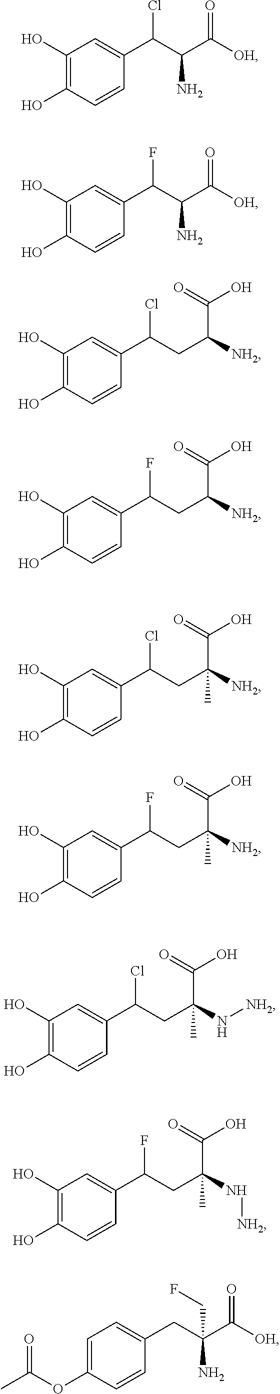

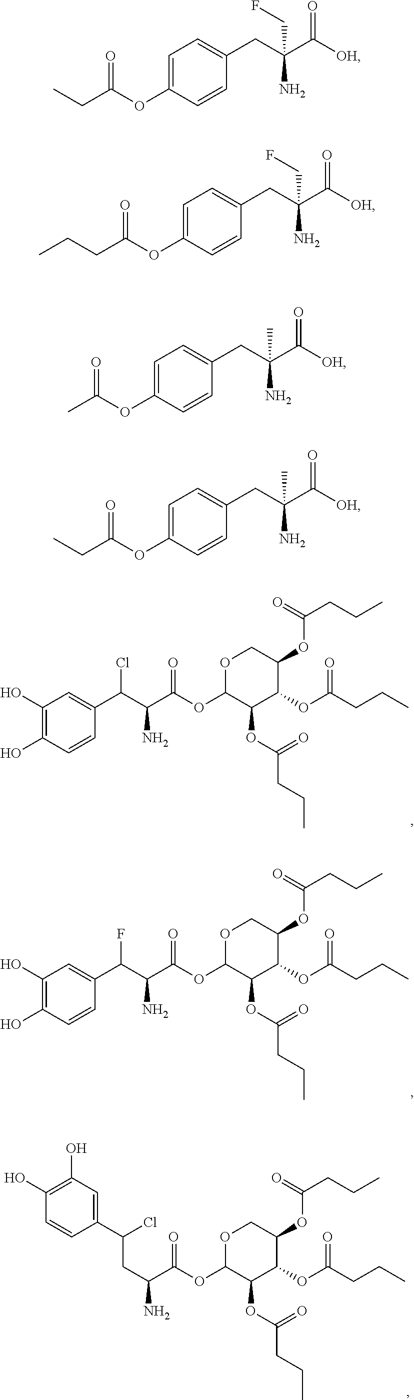

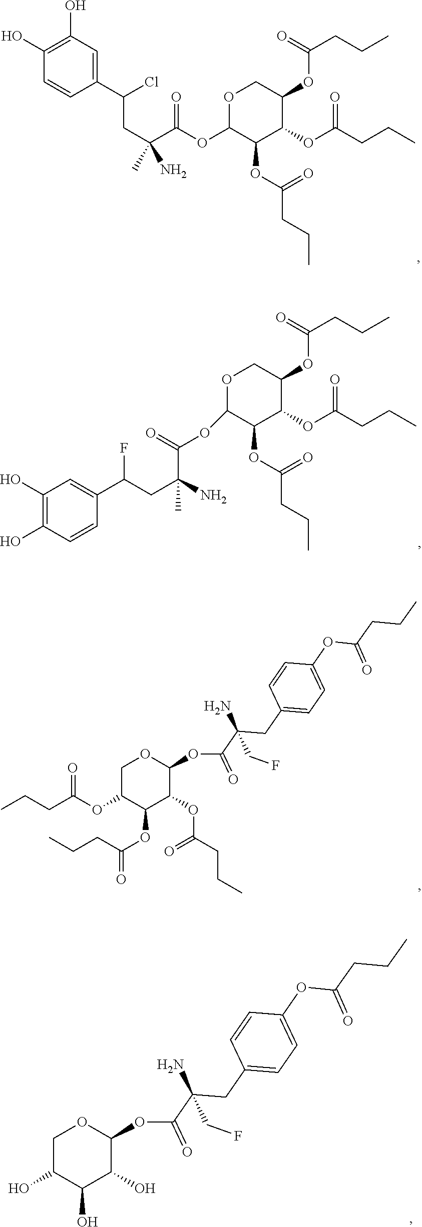

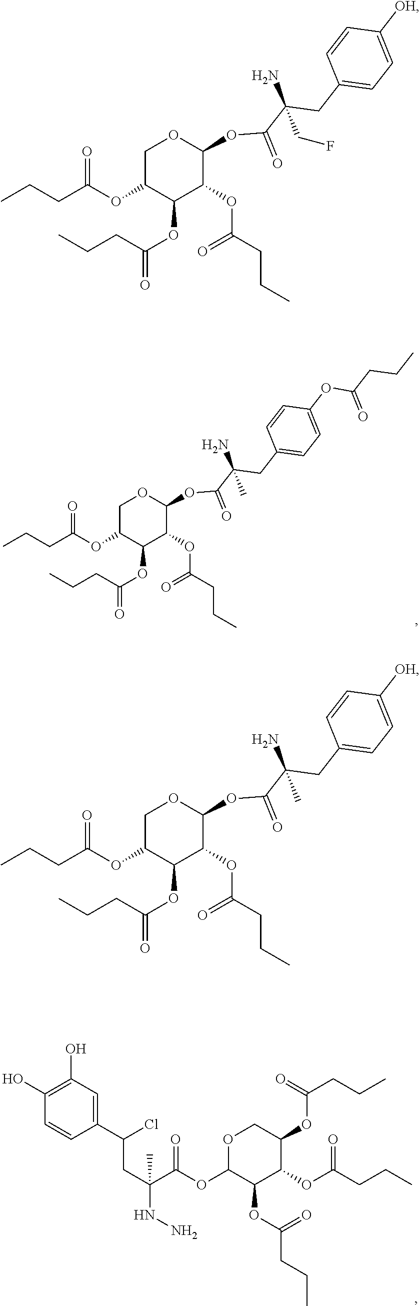

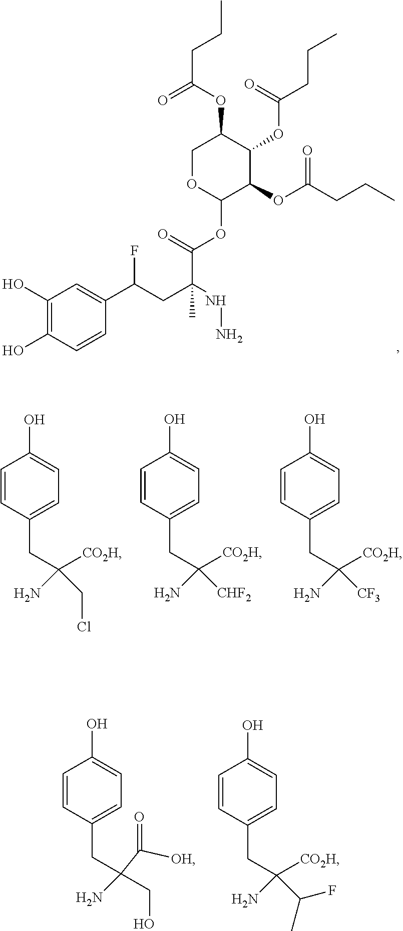 [SMILES: CCCC(=O)O[C@H]1[C@H](OC(=O)CCC)COC(OC(=O)[C@](C)(CC(F)c2ccc(O)c(O)c2)NN)[C@@H]1OC(=O)CCC ; NC(CCl)(Cc1ccc(O)cc1)C(=O)O ; NC(Cc1ccc(O)cc1)(C(=O)O)C(F)F ; NC(Cc1ccc(O)cc1)(C(=O)O)C(F)(F)F ; NC(CO)(Cc1ccc(O)cc1)C(=O)O ; CC(F)C(N)(Cc1ccc(O)cc1)C(=O)O]

and pharmaceutically acceptable salts thereof.

In some embodiments, the tyrosine decarboxylase inhibitor is chosen from:

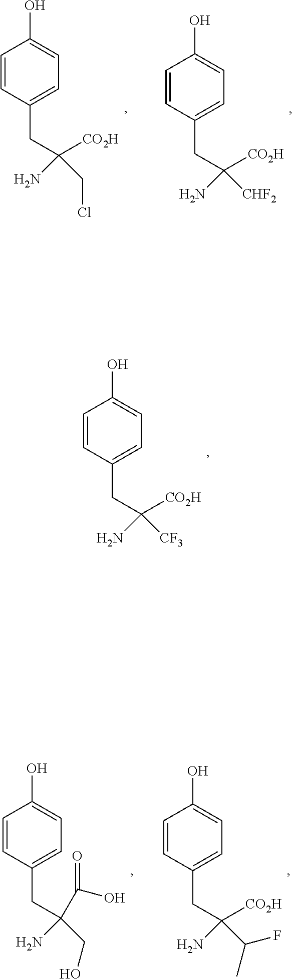 [SMILES: NC(CCl)(Cc1ccc(O)cc1)C(=O)O ; NC(Cc1ccc(O)cc1)(C(=O)O)C(F)F ; NC(Cc1ccc(O)cc1)(C(=O)O)C(F)(F)F ; NC(CO)(Cc1ccc(O)cc1)C(=O)O ; CC(F)C(N)(Cc1ccc(O)cc1)C(=O)O]

and pharmaceutically acceptable salts thereof.

In some embodiments, the tyrosine decarboxylase inhibitor is chosen from:

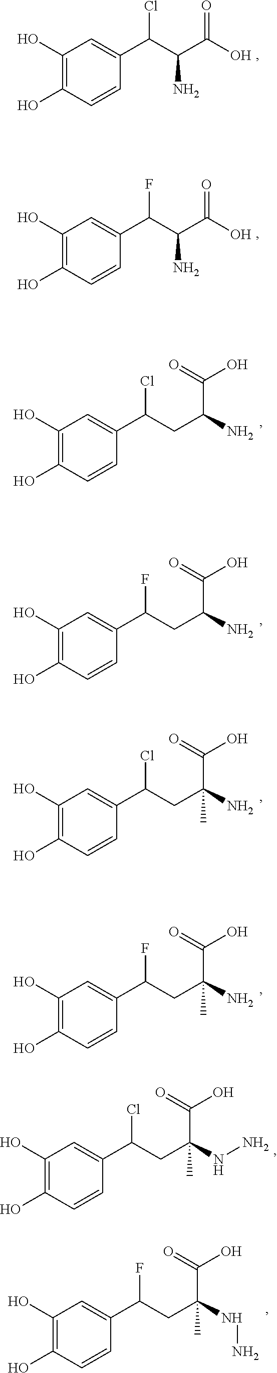

and pharmaceutically acceptable salts thereof.

In some embodiments, the tyrosine decarboxylase inhibitor is chosen from:

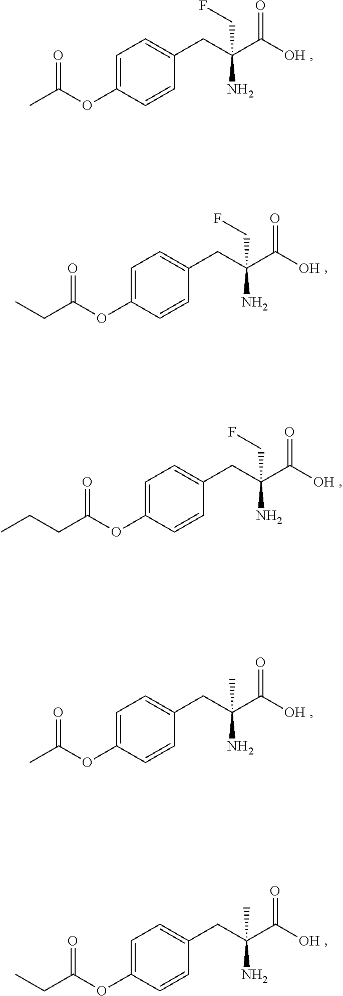

and pharmaceutically acceptable salts thereof.

In some embodiments, the tyrosine decarboxylase inhibitor is chosen from:

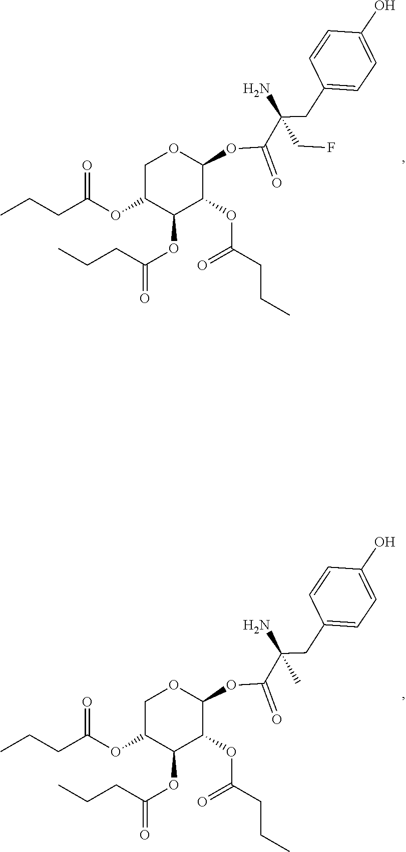

and pharmaceutically acceptable salts thereof.

In some embodiments, the tyrosine decarboxylase inhibitor is chosen from:

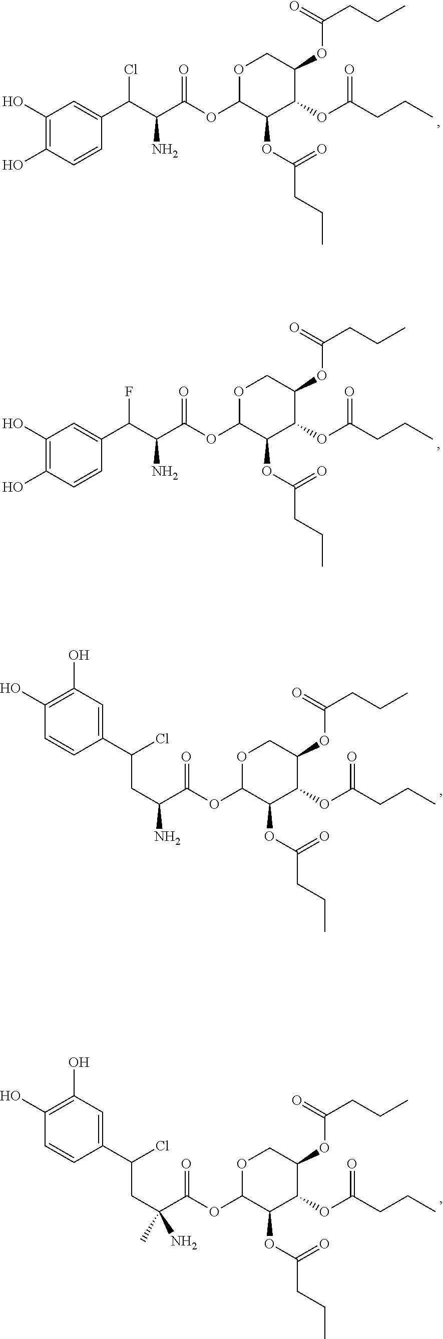 [SMILES: CCCC(=O)O[C@H]1[C@H](OC(=O)CCC)COC(OC(=O)[C@@H](N)C(Cl)c2ccc(O)c(O)c2)[C@@H]1OC(=O)CCC ; CCCC(=O)O[C@H]1[C@H](OC(=O)CCC)COC(OC(=O)[C@@H](N)C(F)c2ccc(O)c(O)c2)[C@@H]1OC(=O)CCC ; CCCC(=O)O[C@H]1[C@H](OC(=O)CCC)COC(OC(=O)[C@@H](N)CC(Cl)c2ccc(O)c(O)c2)[C@@H]1OC(=O)CCC ; CCCC(=O)O[C@H]1[C@H](OC(=O)CCC)COC(OC(=O)[C@@](C)(N)CC(Cl)c2ccc(O)c(O)c2)[C@@H]1OC(=O)CCC]

and pharmaceutically acceptable salts thereof.

In some embodiments, the tyrosine decarboxylase inhibitor is chosen from:

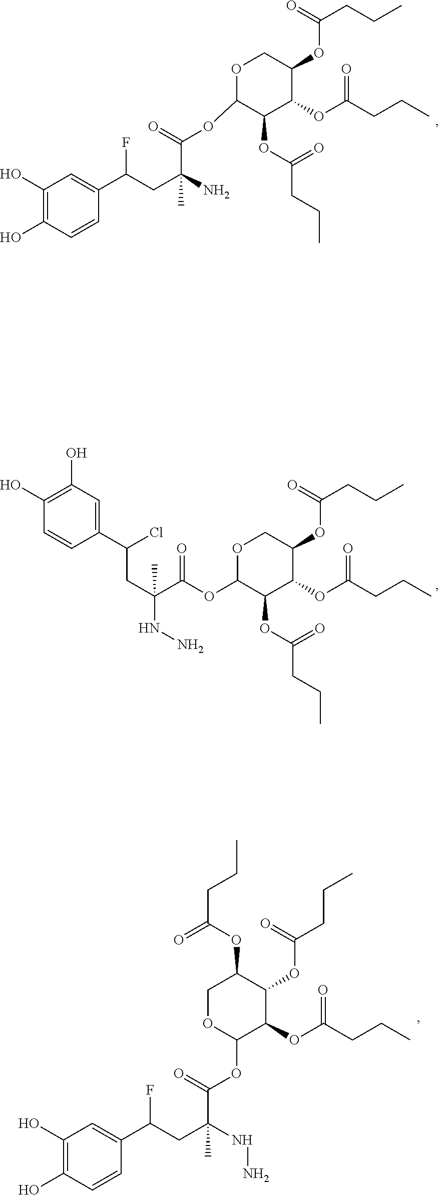 [SMILES: CCCC(=O)O[C@H]1[C@H](OC(=O)CCC)COC(OC(=O)[C@@](C)(N)CC(F)c2ccc(O)c(O)c2)[C@@H]1OC(=O)CCC ; CCCC(=O)O[C@H]1[C@H](OC(=O)CCC)COC(OC(=O)[C@](C)(CC(Cl)c2ccc(O)c(O)c2)NN)[C@@H]1OC(=O)CCC ; CCCC(=O)O[C@H]1[C@H](OC(=O)CCC)COC(OC(=O)[C@](C)(CC(F)c2ccc(O)c(O)c2)NN)[C@@H]1OC(=O)CCC]

and pharmaceutically acceptable salts thereof.

In some embodiments, the tyrosine decarboxylase inhibitor is chosen from:

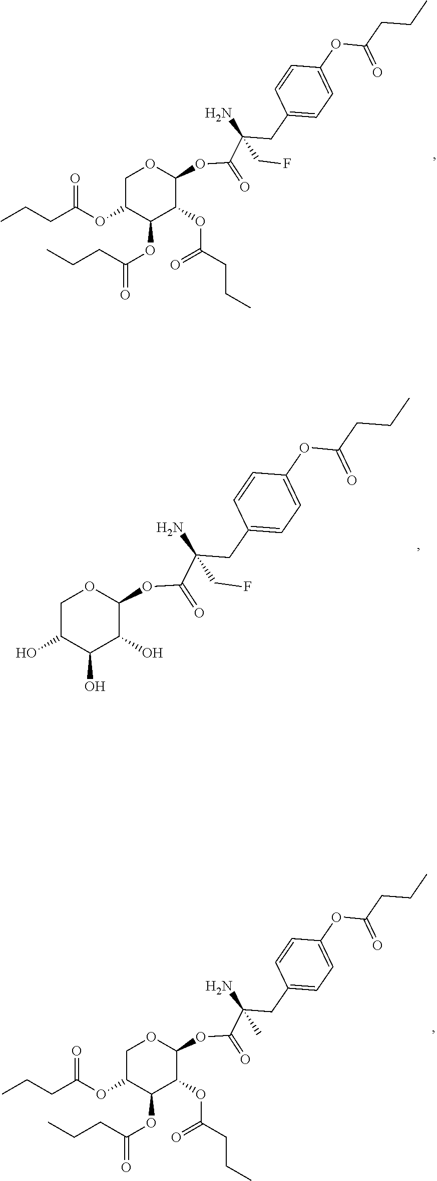

and pharmaceutically acceptable salts thereof.

In some embodiments, the tyrosine decarboxylase inhibitor is chosen from:

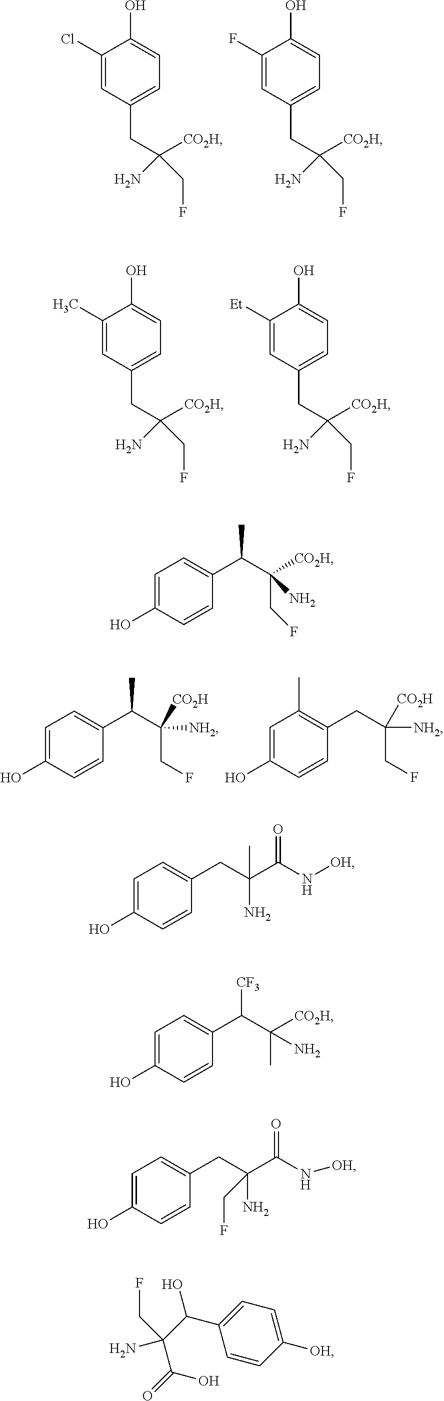

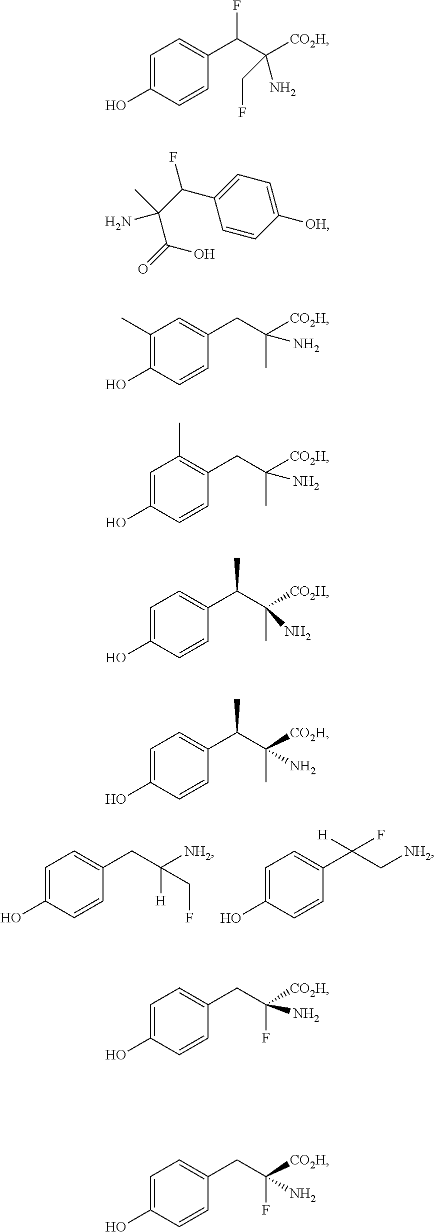

and pharmaceutically acceptable salts thereof.

In some embodiments, the tyrosine decarboxylase inhibitor is chosen from:

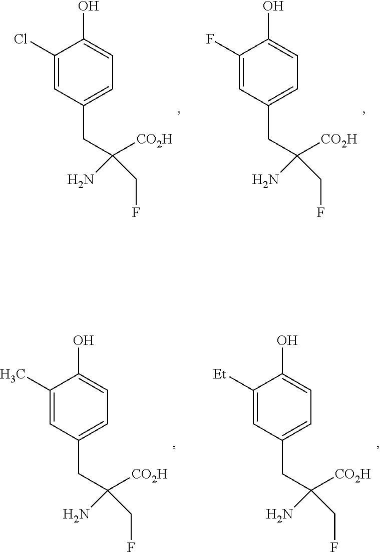 [SMILES: NC(CF)(Cc1ccc(O)c(Cl)c1)C(=O)O ; NC(CF)(Cc1ccc(O)c(F)c1)C(=O)O ; Cc1cc(CC(N)(CF)C(=O)O)ccc1O ; CCc1cc(CC(N)(CF)C(=O)O)ccc1O]

and pharmaceutically acceptable salts thereof.

In some embodiments, the tyrosine decarboxylase inhibitor is chosen from:

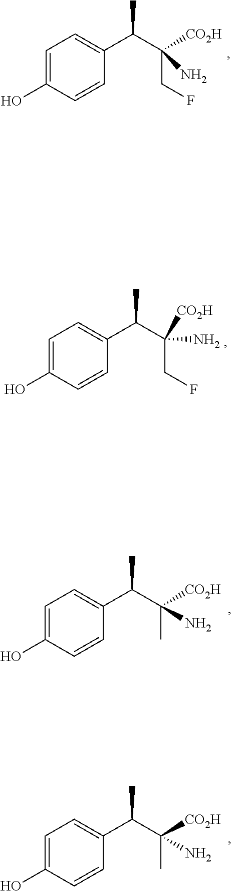 [SMILES: C[C@H](c1ccc(O)cc1)[C@@](N)(CF)C(=O)O ; C[C@H](c1ccc(O)cc1)[C@](N)(CF)C(=O)O ; C[C@H](c1ccc(O)cc1)[C@](C)(N)C(=O)O ; C[C@H](c1ccc(O)cc1)[C@@](C)(N)C(=O)O]

and pharmaceutically acceptable salts thereof.

In some embodiments, the tyrosine decarboxylase inhibitor is chosen from:

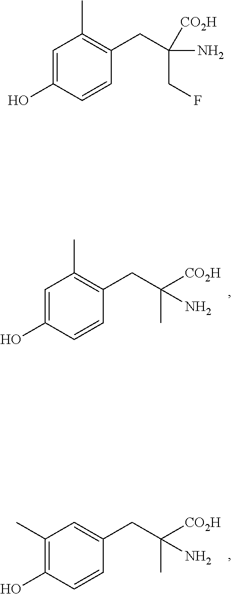 [SMILES: Cc1cc(O)ccc1CC(N)(CF)C(=O)O ; Cc1cc(O)ccc1CC(C)(N)C(=O)O ; Cc1cc(CC(C)(N)C(=O)O)ccc1O]

and pharmaceutically acceptable salts thereof.

In some embodiments, the tyrosine decarboxylase inhibitor is chosen from:

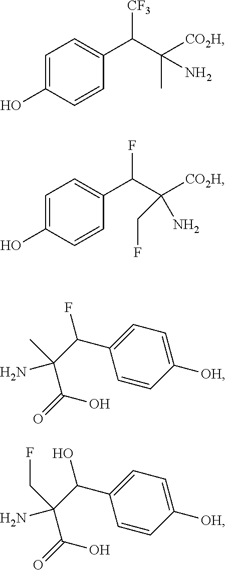

and pharmaceutically acceptable salts thereof.

In some embodiments, the tyrosine decarboxylase inhibitor is chosen from:

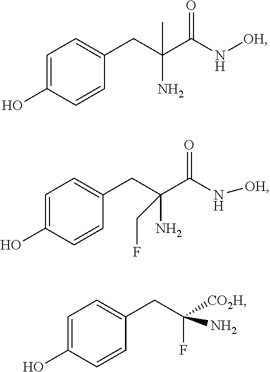 [SMILES: CC(N)(Cc1ccc(O)cc1)C(=O)NO ; NC(CF)(Cc1ccc(O)cc1)C(=O)NO ; N[C@@](F)(Cc1ccc(O)cc1)C(=O)O]

and pharmaceutically acceptable salts thereof.

In some embodiments, the tyrosine decarboxylase inhibitor is chosen from:

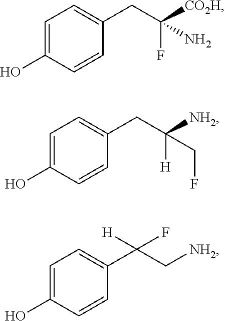

and pharmaceutically acceptable salts thereof.

### EXAMPLES

The following examples provide illustrative embodiments of the disclosure. One of ordinary skill in the art will recognize the numerous modifications and variations that may be performed without altering the spirit or scope of the disclosure. Such modifications and variations are encompassed within the scope of the disclosure. The examples provided do not in any way limit the disclosure.

### Example 1: Preparation of Exemplary Compounds

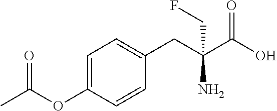

### Compound A: (S)-2-(4-acetoxybenzyl)-2-amino-3-fluoropropanoic acid

(2S)-2-amino-3-fluoro-2-[(4-hydroxyphenyl)methyl]propanoic acid (1 equiv), is treated with Na₂CO₃, acetic anhydride to afford the title compound (S)-2-(4-acetoxybenzyl) amino-3-fluoropropanoic acid.

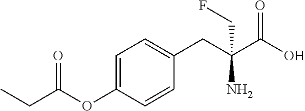

### Compound B: (S)-2-amino-3-fluoro-2-(4-(propionyloxy)benzyl)propanoic acid

(2S)-2-amino-3-fluoro-2-[(4-hydroxyphenyl)methyl]propanoic acid (1 equiv), is treated with Na₂CO₃, propeionic anhydride to afford the title compound (S)-2-amino-3-fluoro-2-(4-(propionyloxy)benzyl)propanoic acid.

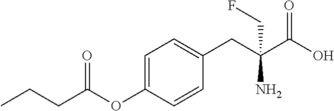

### Compound C: (S)-2-amino-2-(4-(butyryloxy)benzyl)-3-fluoropropanoic acid

(2S)-2-amino-3-fluoro-2-[(4-hydroxyphenyl)methyl]propanoic acid (1 equiv), is treated with Na₂CO₃, butryric anhydride to afford the title compound (S)-2-amino-2-(4-(butyryloxy)benzyl)-3-fluoropropanoic acid.

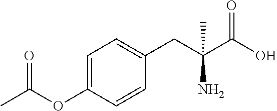

### Compound D: (S)-3-(4-acetoxyphenyl)-2-amino-2-methylpropanoic acid

((2R)-2-amino-3-(4-hydroxyphenyl)-2-methylpropanoic acid (1 equiv), is treated with Na₂CO₃, acetic anhydride to afford the title compound (S)-3-(4-acetoxyphenyl)-2-amino-2-methylpropanoic acid.

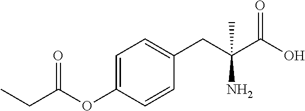

### Compound E: (S)-2-amino-2-methyl-3-(4-(propionyloxy)phenyl)propanoic acid

(((2R)-2-amino-3-(4-hydroxyphenyl)-2-methylpropanoic acid (1 equiv), is treated with Na₂CO₃, propionic anhydride to afford the title compound (S)-2-amino-2-methyl-3-(4-(propionyloxy)phenyl)propanoic acid.

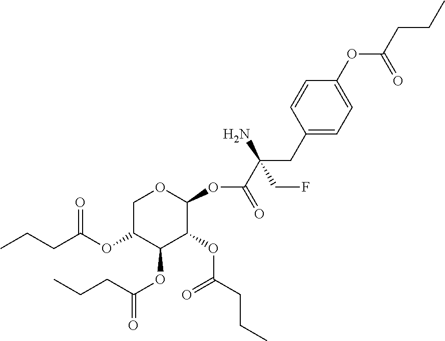

### Compound F: (2S,3R,4S,5R)-2-(((S)-2-amino-2-(4-(butyryloxy)benzyl) fluoropropanoyl)oxy)tetrahydro-2H-pyran-3,4,5-triyltributyrate

(2S)-2-amino-3-fluoro-2-[(4-hydroxyphenyl)methyl]propanoic acid (1 equiv), is treated with 1 equiv of Na₂CO₃ and butryric anhydride and the corresponding butyric acid is DCC coupled to (2S,3S,4R,5S)-2-hydroxytetrahydro-2H-pyran-3,4,5-triyl tributyrate (which can be synthesized from (2S,3S,4R,5S)-tetrahydro-2H-pyran-2,3,4,5-tetraol) to afford the title compound (2S,3R,4S,5R)-2-(((S)-2-amino-2-(4-(butyryloxy)benzyl)-3-fluoropropanoyl)oxy)tetrahydro-2H-pyran-3,4,5-triyl tributyrate.

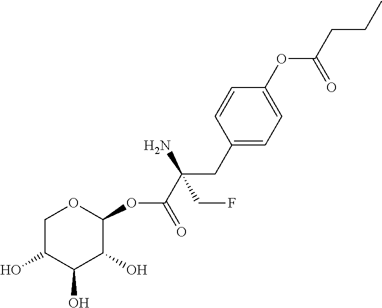

### Compound G: 4-((S)-2-amino-2-(fluoromethyl)-3-oxo-3-(((2S,3R,4S,5R)-3,4,5-trihydroxytetrahydro-2H-pyran-2-yl)oxy)propyl)phenyl butyrate

(2S)-2-amino-3-fluoro-2-[(4-hydroxyphenyl)methyl]propanoic acid (1 equiv) is treated with 1 equiv of Na₂CO₃ and butryric anhydride, and the corresponding butyric acid is DCC coupled to (2S,3S,4R,5S)-2-hydroxytetrahydro-2H-pyran-3,4,5-triyl acetate (which can be synthesized from (2S,3S,4R,5S)-tetrahydro-2H-pyran-2,3,4,5-tetraol). This material is then treated with dilute lithium hydroxide in water to afford the title compound.

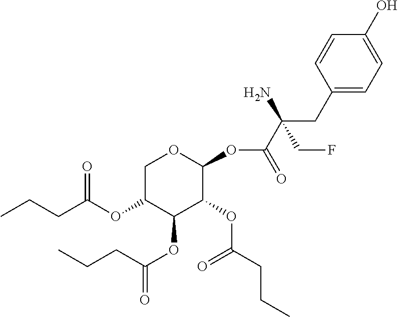

### Compound H: (2S,3R,4S,5R)-2-(((S)-2-amino-3-fluoro-2-(4-hydroxybenzyl)propanoyl)oxy)tetrahydro-2H-pyran-3,4,5-triyltributyrate

(2S)-2-amino-3-fluoro-2-[(4-hydroxyphenyl)methyl]propanoic acid (1 equiv), is treated with 1 equiv of BnBr, K₂CO₃ in THF and the corresponding benzyl acid is DCC coupled to (2S,3S,4R,5S)-2-hydroxytetrahydro-2H-pyran-3,4,5-triyl tributyrate (which can be synthesized from (2S,3S,4R,5S)-tetrahydro-2H-pyran-2,3,4,5-tetraol) and hydrogenated with Pd(OH)₂/H₂, to afford the title compound (2S,3R,4S,5R)-2-(((S)-2-amino-3-fluoro-2-(4-hydroxybenzyl)propanoyl)oxy)tetrahydro-2H-pyran-3,4,5-triyl tributyrate.

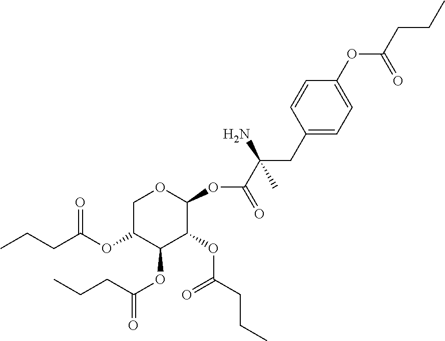

### Compound I: (2S,3R,4S,5R)-2-(((S)-2-amino-3-(4-(butyryloxy)phenyl)-2-methylpropanoyl)oxy)tetrahydro-2H-pyran-3,4,5-triyl tributyrate

((2R)-2-amino-3-(4-hydroxyphenyl)-2-methylpropanoic acid (1 equiv), is treated with 1 equiv of Na₂CO₃ and butyric anhydride and the corresponding carboxylic acid is DCC coupled to (2S,3S,4R,5S)-2-hydroxytetrahydro-2H-pyran-3,4,5-triyl tributyrate (which can be synthesized from (2S,3S,4R,5S)-tetrahydro-2H-pyran-2,3,4,5-tetraol) to afford the title compound (2S,3R,4S,5R)-2-(((S)-2-amino-3-(4-(butyryloxy)phenyl)-2-methylpropanoyl)oxy)tetrahydro-2H-pyran-3,4,5-triyl tributyrate.

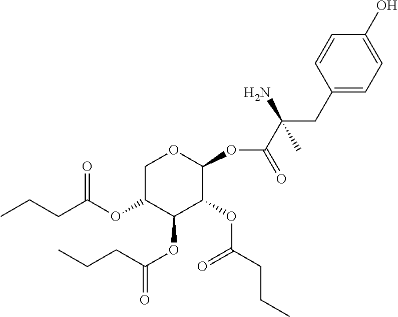

### Compound J: (2S,3R,4S,5R)-2-(((S)-2-amino-3-(4-hydroxyphenyl)-2-methylpropanoyl)oxy)tetrahydro-2H-pyran-3,4,5-triyltributyrate

((2R)-2-amino-3-(4-hydroxyphenyl)-2-methylpropanoic acid (1 equiv), is treated with 1 equiv of BnBr, K₂CO₃ in THF and the corresponding benzyl acid is DCC coupled to (2S,3S,4R,5S)-2-hydroxytetrahydro-2H-pyran-3,4,5-triyl tributyrate (which can be synthesized from (2S,3S,4R,5S)-tetrahydro-2H-pyran-2,3,4,5-tetraol) and hydrogenated with Pd(OH)₂/H₂, to afford the title compound (2S,3R,4S,5R)-2-(((S)-2-amino-3-(4-hydroxyphenyl)-2-methylpropanoyl)oxy)tetrahydro-2H-pyran-3,4,5-triyl tributyrate.

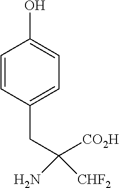 [SMILES: NC(Cc1ccc(O)cc1)(C(=O)O)C(F)F]

### Compound K: 2-amino-3,3-difluoro-2-(4-hydroxybenzyl)propanoic acid

Step 1:

To a solution of LDA (2 M, 60.18 mL, 2 eq, THF) in THF (50 mL) was added 2-(4-methoxyphenyl)acetic acid (10 g, 60.18 mmol, 1 eq) in THF (50 mL) at −70° C. and the mixture was stirred at 0° C. for 3 h. Then the mixture was cooled to −70° C. and ethyl 2,2-difluoroacetate (8.21 g, 66.20 mmol, 1.1 eq) in THF (50 mL) was added to the mixture at −70° C. and stirred at −70° C. for 2 h. The reaction mixture was quenched by addition 1 N HCl 150 mL at 0° C., and then extracted with EtOAc 300 mL (100 mL*3). The combined organic layers were washed with sat. NaHCO₃ 150 mL (50 mL*3) and brine 100 mL (50 mL*2), dried over Na₂SO₄, filtered and the filtrate was concentrated under reduced pressure to give a residue. The residue was purified by column chromatography (SiO₂, Petroleum ether/Ethyl acetate=I/O to 0/1) to give 1,1-difluoro-3-(4-methoxyphenyl)propan-2-one (2.3 g, 11.49 mmol, 19.09% yield) as yellow liquid.

Step 2:

A mixture of 1,1-difluoro-3-(4-methoxyphenyl)propan-2-one (2.3 g, 11.49 mmol, 1 eq) and (NH₄)₂CO₃ (5.19 g, 54.00 mmol, 5.77 mL, 4.7 eq) in EtOH (12 mL) and H₂O (8 mL) was stirred at 55° C., degassed and purged with N₂ 3 times, and then NaCN (608.12 mg, 12.41 mmol, 1.08 eq) was added to the mixture and stirred at 55° C. for 21 h under N₂ atmosphere. Then the mixture was stirred at 90° C. for 0.5 h. The reaction mixture was diluted with H₂O 20 mL and extracted with EtOAc 120 mL (20 mL*6). The combined organic layers were dried over Na₂SO₄, filtered and the filtrate was concentrated under reduced pressure to give a residue. The residue was purified by column chromatography (SiO₂, Petroleum ether/Ethyl acetate=I/O to 0/1) to give 5-(difluoromethyl)-5-[(4-methoxyphenyl)methyl]imidazolidine-2,4-dione (1.9 g, 5.98 mmol, 52.02% yield, 85% purity) as a yellow solid.

Step 3:

A mixture of 5-(difluoromethyl)-5-[(4-methoxyphenyl)methyl]imidazolidine-2,4-dione (1.8 g, 6.66 mmol, 1 eq) in aq. HBr (18 mL, 48%) was degassed and purged with N₂ 3 times, and then the mixture was stirred at 110° C. for 5 h under N₂ atmosphere. The reaction mixture was washed with EtOAc 30 mL (10 mL*3). The aqueous phase was concentrated under reduced pressure to give a residue. The residue was used sat. NaHCO₃ to adjust pH to 7-8, then 6 M HCl was added to the mixture and the pH was adjusted to 3-4. The residue was purified by prep-HPLC (column: Welch Xtimate C18 150*25 mm*5 μm; mobile phase: [water (0.04% HCl)-ACN]; B %: 1%-5%, 10 min) to give 2-amino-3,3-difluoro-2-[(4-hydroxyphenyl)methyl]propanoic acid (56 mg, 202.95 μmol, 3.05% yield, 97% purity, HCl) as a white solid. LC-MS m/z=232.1. ¹H NMR (400 MHz, DMSO-d6) δ 9.26 (s, 1H), 7.37 (br s, 1H), 7.06 (d, J=9.6 Hz, H), 6.63 (d, J=9.0 Hz, 2H), 6.12 (t, J=32.8 Hz, 1H), 3.03 (d, J=13.6 Hz, 1H), 2.67 (d, J=13.6 Hz, 1H).

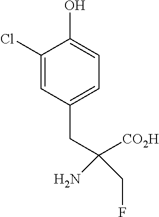 [SMILES: NC(CF)(Cc1ccc(O)c(Cl)c1)C(=O)O]

### Compound L: 2-amino-2-(3-chloro-4-hydroxybenzyl)-3-fluoropropanoic acid

Step 1:

To a solution of 4-(bromomethyl)-2-chloro-1-methoxy-benzene (3 g, 12.74 mmol, 1 eq) and 2-(benzhydrylideneamino)acetonitrile (1.84 g, 8.34 mmol, 6.55e-1 eq) in DCM (30 mL) was added benzyl(trimethyl)ammonium chloride (189.24 mg, 1.02 mmol, 176.86 μL, 0.08 eq), then aq. NaOH (10 M, 1.91 mL, 1.5 eq) was added dropwise at 0° C. The mixture was warmed to 25° C. and stirred for 12 h. TLC indicated Reactant was consumed completely and two new spots formed. The reaction mixture was concentrated under reduced pressure to remove DCM (30 mL). The residue was purified by column chromatography (SiO₂, Petroleum ether/Ethyl acetate=20/1 to 10/1). Compound 2-(benzhydrylideneamino)-3-(3-chloro-4-methoxy-phenyl)propanenitrile (2.4 g, 6.40 mmol, 50.26% yield) was obtained as a yellow oil.

Step 2:

To a solution of 2-(benzhydrylideneamino)-3-(3-chloro-4-methoxy-phenyl)propanenitrile (2.4 g, 6.40 mmol, 1 eq) in THF (25 mL) was added fluoro(iodo)methane (5.12 g, 32.01 mmol, 5 eq) and KOtBu (3.59 g, 32.01 mmol, 5 eq). The mixture was stirred at 25° C. for 1 h. LC-MS showed 2-(benzhydrylideneamino)-3-(3-chloro-4-methoxy-phenyl)propanenitrile was consumed completely and the desired MS was detected. The reaction mixture was filtered and filtrate concentrated under reduced pressure to give a residue. The residue was purified by prep-TLC (SiO₂, Petroleum Ether: Ethyl Acetate=3:1). Compound 2-(benzhydrylideneamino)-2-[(3-chloro-4-methoxy-phenyl) methyl]-3-fluoro-propanenitrile (800 mg, 1.97 mmol, 30.71% yield) was obtained as yellow oil.

Step 3:

The mixture of 2-(benzhydrylideneamino)-2-[(3-chloro-4-methoxy-phenyl)methyl]-3-fluoro-propanenitrile (800 mg, 1.97 mmol, 1 eq) in aq. HBr (331.43 mg, 1.97 mmol, 222.44 μL, 48% purity, 1 eq) was stirred at 110° C. for 12 h. LC-MS showed 2-(benzhydrylideneamino)-2-[(3-chloro-4-methoxy-phenyl)methyl]-3-fluoro-propanenitrile was consumed completely. The residue was diluted with H₂O (10 mL) and extracted with EtOAc 15 mL (5 mL*3). The H₂O phase was freeze-dried. The residue was purified by prep-HPLC (column: Phenomenex luna C18 250*50 mm*10 μm; mobile phase: [water (0.1% TFA)-ACN]; B %: 1%-20%, 10 min) to give the crude product. The crude product in H₂O (3 mL) was adjusted pH to 7-8 with sat. NaHCO₃aq. then adjusted the pH to 3-4 with 6 M HCl. The aqueous phase was purified by purified by prep-HPLC (column: Phenomenex luna C18 250*50 mm*10 μm; mobile phase: [water (0.05% HCl)-ACN]; B %: 1%-20%, 10 min). Compound 2-amino-2-[(3-chloro-4-hydroxy-phenyl)methyl]-3-fluoro-propanoic acid (150 mg, 527.96 mol, 54.48% yield, HCl) was obtained as a white solid. LC-MS m/z=248.0. ¹H NMR (400 MHz, DMSO-d6) δ 10.29 (br s, 1H), 7.21 (d, J=2.0 Hz, 1H), 7.00 (dd, J=8.4, 2.0 Hz, 1H), 6.92 (d, J=8.4 Hz, 1H), 4.80 (dd, J=46.8 9.8 Hz, 1H), 4.70 (dd, J=46.8, 9.8 Hz, 1H), 2.99 (d, J=14.4 Hz, 1H), 2.94 (d, J=14.4 Hz, 1H).

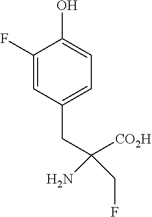 [SMILES: NC(CF)(Cc1ccc(O)c(F)c1)C(=O)O]

### Compound M: 2-amino-3-fluoro-2-(3-fluoro-4-hydroxybenzyl)propanoic acid

Step 1:

To a solution of 4-(bromomethyl)-2-fluoro-1-methoxy-benzene (3 g, 13.70 mmol, 1.51 eq) and 2-(benzhydrylideneamino)acetonitrile (2 g, 9.08 mmol, 1 eq) in DCM (30 mL) was added benzyl(trimethyl)ammonium chloride (134.89 mg, 726.39 μmol, 126.06 μL, 0.08 eq), then aq. NaOH (10 M, 1.36 mL, 1.5 eq) was added dropwise at 0° C. The mixture was warmed to 50° C. and stirred for 12 h. TLC indicated 4-(bromomethyl)-2-fluoro-1-methoxy-benzene was not consumed completely and two new spots formed. The reaction mixture was concentrated under reduced pressure to remove DCM (30 mL). The residue was purified by column chromatography (SiO₂, Petroleum ether/Ethyl acetate=10/1 to 5/1). Compound 2-(benzhydrylideneamino)-3-(3-fluoro-4-methoxy-phenyl)propanenitrile (2.6 g, 7.25 mmol, 79.89% yield) was obtained as yellow oil.

Step 2:

To a solution of 2-(benzhydrylideneamino)-3-(3-fluoro-4-methoxy-phenyl)propanenitrile (2.6 g, 7.25 mmol, 1 eq) and fluoro(iodo)methane (5.80 g, 36.27 mmol, 5 eq) in THF (30 mL) was added KOtBu (4.07 g, 36.27 mmol, 5 eq, solid). The mixture was stirred at 25° C. for 1.5 h. LC-MS showed 2-(benzhydrylideneamino)-3-(3-fluoro-4-methoxy-phenyl)propanenitrile was consumed completely and desired MS was detected. The reaction mixture was filtered and filtrate concentrated under reduced pressure to give a residue. The residue was purified by column chromatography (SiO₂, Petroleum ether/Ethyl acetate=15/1 to 5/1). Compound 2-(benzhydrylideneamino)-3-fluoro-2-[(3-fluoro-4-methoxy-phenyl)methyl]propanenitrile (1 g, 2.56 mmol, 35.31% yield) was obtained as a yellow oil.

Step 3:

The mixture of 2-(benzhydrylideneamino)-3-fluoro-2-[(3-fluoro-4-methoxy-phenyl) methyl]propanenitrile (600 mg, 1.54 mmol, 1 eq) in aq. HBr (4.47 g, 26.52 mmol, 3 mL, 48% purity, 17.26 eq) was stirred at 110° C. for 12 h. LC-MS showed 2-(benzhydrylideneamino)-3-fluoro-2-[(3-fluoro-4-methoxy-phenyl)methyl]propanenitrile was consumed completely and one main peak with desired MS was detected. The reaction mixture was concentrated under reduced pressure to remove HBr (3 mL). Then pH was adjusted to 7-8 by saturated NaHCO₃ aqueous and then pH was adjusted to 7-8 with 6 N HCl. The aqueous phase was purified by prep-HPLC (column: Phenomenex luna C18 250*50 mm*10 μm; mobile phase: [water (0.05% HCl)-ACN]; B %: 1%-10%, 10 min). Compound 2-amino-3-fluoro-2-[(3-fluoro-4-hydroxy-phenyl)methyl]propanoic acid (77 mg, 277.61 μmol, 18.06% yield, 96.5% purity, HCl) was obtained as a white solid. LC-MS m/z=232.0. ¹H NMR (400 MHz, DMSO-d6) δ 10.01 (br s, 1H), 8.85 (br s, 3H), 7.04 (d, 12.4 Hz, 1H), 6.95 (t, J=8.4 Hz, 1H), 6.86 (d, J=8.4 Hz), 4.89 (dd, J=46.6, 10 Hz, 1H), 4.71 (dd, J=46.6, 10 Hz, 1H, 3.12-3.04 (m, 2H).

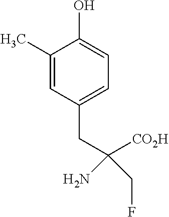 [SMILES: Cc1cc(CC(N)(CF)C(=O)O)ccc1O]

### Compound N: 2-amino-3-fluoro-2-(4-hydroxy-3-methylbenzyl)propanoic acid

Step 1:

To a solution of 4-(chloromethyl)-1-methoxy-2-methyl-benzene (3 g, 17.58 mmol, 1 eq) in acetone (30 mL) was added NaI (5.27 g, 35.16 mmol, 2 eq) at 25° C. Then the mixture was stirred at 25° C. for 10 h. The reaction mixture was filtered and the filtrate was concentrated under reduced pressure to give a residue. The residue was diluted with H₂O 10 mL and extracted with EtOAc 30 mL (10 mL*3). The combined organic layers were washed with brine 20 mL (10 mL*2) and aq. sodium thiosulfate 20 mL (10 mL*2), dried over Na₂SO₄, filtered and the filtrate was concentrated under reduced pressure to give a residue. The residue was purified by column chromatography (SiO₂, Petroleum ether/Ethyl acetate=I/O to 1/1) to give 4-(iodomethyl)-1-methoxy-2-methyl-benzene (4 g, 15.26 mmol, 86.81% yield) as a yellow liquid.

Step 2:

To a solution of 4-(iodomethyl)-1-methoxy-2-methyl-benzene (4 g, 15.26 mmol, 1.2 eq), 2-(benzhydrylideneamino)acetonitrile (2.80 g, 12.72 mmol, 1 eq) and N,N,N-trimethyl-1-phenylmethanaminium chloride (236.17 mg, 1.27 mmol, 220.72 μL, 0.1 eq) in DCM (40 mL) was added aq. NaOH (10 M, 2.29 mL, 1.8 eq) at 0° C. The mixture was stirred at 25° C. for 10 h and stirred at 50° C. for 24 h. The reaction mixture was filtered and the filtrate was concentrated under reduced pressure to give a residue. The residue was diluted with H₂O 15 mL and extracted with EtOAc 60 mL (20 mL*3). The combined organic layers were dried over Na₂SO₄, filtered and the filtrate was concentrated under reduced pressure to give a residue. The residue was purified by prep-HPLC (column: Agela DuraShell C18 250*80 mm*10 μm; mobile phase: [water (0.04% NH₃H₂O+10 mM NH₄HCO₃)-ACN]; B %: 55%-85%, 20 min) to give 2-(benzhydrylideneamino)-3-(4-methoxy-3-methyl-phenyl)propanenitrile (1.9 g, 5.36 mmol, 42.15% yield) as yellow oil.

Step 3:

To a solution of 2-(benzhydrylideneamino)-3-(4-methoxy-3-methyl-phenyl)propanenitrile (0.5 g, 1.41 mmol, 1 eq) in THF (10 mL) was added t-BuOK (791.45 mg, 7.05 mmol, 5 eq) and fluoro(iodo)methane (2.26 g, 14.11 mmol, 10 eq). Then the mixture was stirred at 25° C. for 1 h. The reaction mixture was filtered and the filtrate was concentrated under reduced pressure to give a residue. The residue was purified by column chromatography (SiO₂, Petroleum ether/Ethyl acetate=I/O to 5/1) to give 2-(benzhydrylideneamino)-2-(fluoromethyl)-3-(4-methoxy-3-methyl-phenyl)propanenitrile (0.45 g, 1.16 mmol, 82.54% yield) as yellow oil.

Step 4:

A mixture of 2-(benzhydrylideneamino)-2-(fluoromethyl)-3-(4-methoxy-3-methyl-phenyl) propanenitrile (0.44 g, 1.14 mmol, 1 eq) in aq. HBr (8 mL, 48%) was degassed and purged with N₂ 3 times, and then the mixture was stirred at 110° C. for 10 h under N₂ atmosphere. The reaction mixture was washed with EtOAc 30 mL (10 mL*3). The aqueous phase was concentrated under reduced pressure to give a residue. The residue was purified by prep-HPLC (column: Luna Omega 5u Polar C18 100 Å; mobile phase: [water (0.04% HCl)-ACN]; B %: 1%-10%, 7 min) to give the product. The product in H₂O (2 mL) was adjusted pH to 7-8 with sat. NaHCO₃aq. then adjusted the pH to 3-4 with 6 M HCl. The aqueous phase was purified by prep-HPLC (column: Luna Omega 5u Polar C18 100 Å; mobile phase: [water (0.04% HCl)-ACN]; B %: 1%-15%, 7 min) to give 2-amino-2-(fluoromethyl)-3-(4-hydroxy-3-methyl-phenyl)propanoic acid (54 mg, 204.78 μmol, 20.87% yield, 100% purity, HCl) as a white solid. LC-MS m/z=228.1. ¹H NMR (400 MHz, CD₃OD) δ 6.96 (s, 1H), 6.89 (d, J=8.0 Hz, 1H), 6.71 (d, J=8.0 Hz, 1H), 4.89 (dd, J=47.0, 10.0 Hz, 1H), 4.65 (dd, J=47.0, 10.0 Hz, 1H), 3.17 (d, J=7.2 Hz, 1H), (2.96, J=14.2 Hz, 1H), 2.17 (s, 3H).

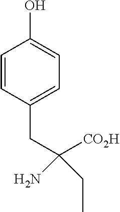

### Compound M: 2-amino-2-(4-hydroxybenzyl)butanoic acid

Step 1:

To a mixture of tert-butyl 2-amino-3-(4-hydroxyphenyl)propanoate (30 g, 126.43 mmol, 1 eq) and diphenylmethanone (23.04 g, 126.43 mmol, 1 eq) in Toluene (300 mL) was added TsOH (2.18 g, 12.64 mmol, 0.1 eq). The mixture was stirred at 120° C. for 48 h and the water removed by Dean-Stark trap. TLC (PE:EtOAc=5:1) indicated a little starting material remained, and one major new spot was detected. The reaction mixture was concentrated to give a residue. The residue was purified by column chromatography (SiO₂, Petroleum ether/Ethyl acetate=10/1 to 5/1). Compound tert-butyl 2-(benzhydrylideneamino)-3-(4-hydroxyphenyl) propanoate (8.5 g, 21.17 mmol, 16.75% yield) was obtained as a yellow oil.

Step 2:

To the solution of tert-butyl (2S)-2-(benzhydrylideneamino)-3-(4-hydroxyphenyl)propanoate (12.5 g, 31.13 mmol, 1 eq) in THF (125 mL) was added NaH (1.62 g, 40.47 mmol, 60% purity, 1.3 eq) at 0° C. The mixture was stirred at 0° C. for 0.5 h. Then MOMCI (3.26 g, 40.47 mmol, 3.07 mL, 1.3 eq) was added drop-wise to the mixture at 0° C. The mixture was allowed to warm to 25° C. and stirred at 25° C. for 2.5 h. TLC (PE:EtOAc=5:1) indicated the starting material was consumed completely and one new spot formed. The mixture was poured into sat. NaHCO₃ (100 mL) at 0-5° C. The aqueous phase was extracted with EtOAc (100 mL*3). The combined organic layers were dried over Na₂SO₄, filtered and concentrated in vacuum. Compound tert-butyl (2S)-2-(benzhydrylideneamino)-3-[4-(methoxymethoxy)phenyl]propanoate (10 g, 22.44 mmol, 72.09% yield) was obtained as a yellow oil.

Step 3:

To a solution of tert-butyl (2S)-2-(benzhydrylideneamino)-3-[4-(methoxymethoxy)phenyl] propanoate (2.00 g, 4.49 mmol, 1 eq) in THF (40 mL) and HMPA (7.45 g, 41.57 mmol, 7.30 mL, 9.26 eq) was added dropwise LDA (2 M, 15.71 mL, 7 eq) at −70° C. under N₂. The mixture was stirred at −70° C. for 0.5 h. Then CH₃CH₂I (7.00 g, 44.89 mmol, 3.59 mL, 10 eq) was added drop-wise to the above mixture at −70° C. The reaction mixture was allowed to warm to 25° C. and stirred at 25° C. for 1.5 h. TLC (PE:EtOAc=5:1) indicated the starting material was consumed completely and one new spot formed. The reaction was clean according to TLC. The reaction mixture was quenched by sat. NaHCO₃ 100 mL at 0° C. The organic phase was separated, washed with EtOAc (50 mL*3), dried over NaSO₄, filtered and concentrated under reduced pressure to give a residue. The residue was purified by column chromatography (SiO₂, Petroleum ether/Ethyl acetate=20/1 to 8/1). Compound tert-butyl 2-(benzhydrylideneamino)-2-[[4-(methoxymethoxy) phenyl]methyl] butanoate (1 g, 2.11 mmol, 47.04% yield) was obtained as a yellow oil.

Step 4:

To a solution of tert-butyl 2-(benzhydrylideneamino)-2-[[4-(methoxymethoxy)phenyl]methyl] butanoate (1.03 g, 2.17 mmol, 1 eq) in THF (30 mL) was added aq. citric acid (21.81 g, 5.68 mmol, 21.83 mL, 5% purity, 2.61 eq) and the reaction was stirred at 25° C. for 6 h. LC-MS (ET28600-23-P1A, RT=2.371 min) showed the starting material was consumed completely. The mixture was diluted with EtOAc (30 mL) and the mixture was extracted with EtOAc (60 mL*3). The combined organic phases were dried with anhydrous Na₂SO₄, filtered and concentrated in vacuum. The residue was purified by prep-HPLC (HPLC: ET28600-23-P1A, RT=2.522 min, 89.8% purity; Kromasil C18 (250*50 mm*10 μm); mobile phase: [water (10 mM NH₄HCO₃)-ACN]; B %: 30%-60%, 10 min) to give desired compound. Compound tert-butyl 2-amino-2-[[4-(methoxymethoxy)phenyl]methyl]butanoate (0.25 g, 808.02 μmol, 37.15% yield) was obtained as a yellow oil. The product was detected by ¹H NMR (ET28600-23-P1A, MeOD).

Step 5:

To the tert-butyl 2-amino-2-[[4-(methoxymethoxy) phenyl]methyl]butanoate (0.25 g, 808.02 μmol, 1 eq) in dioxane (3 mL) was added aq. HCl (2.5 M, 6.46 mL, 20 eq). The mixture was stirred at 60° C. for 3 h. LC-MS (ET28600-33-P1B, product: M+1=210, RT=0.877 min) showed the starting material was consumed completely. The reaction mixture on notebook page ET28600-24-P1 was combined to ET28600-33-P1 for work up. The reaction mixture was partitioned between water (20 mL) and EtOAc (30 mL). The organic phase was separated, washed with sat. NaHCO₃ (5 mL*3), dried over though Na₂SO₄, filtered and concentrated under reduced pressure to give a residue. The residue was purified by prep-HPLC (HPLC: ET28600-33-P1A, RT=1.772 min; column: Waters Atlantis T3 150*30 mm*5 μm; mobile phase: [water (0.05% HCl)-ACN]; B %: 1%-30%, 12 min) to give desired compound as a white solid. Total 106 mg of 2-amino-2-[(4-hydroxyphenyl) methyl] butanoic acid (HCl salt) was obtained (ET28600-33-P1&ET28600-24-P1, combined together) as a white solid. LC-MS m/z=210.1. ¹H NMR (400 MHz, CD₃OD) δ 7.08 (d, J=8.4 Hz, 1H), 6.78 (d, J=8.4 Hz, 1H), 3.22 (d, J=14.4 Hz, 1H) 2.99 (d, J=14.4 Hz, 1H), 2.12-2.07 (m, 1H), 1.90-1.84 (m, 1H), 1.04 (t, J=7.2 Hz, 1H).

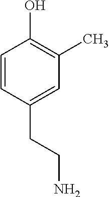

### Compound P: 4-(2-aminoethyl)-2-methylphenol

Step 1:

To a solution of 4-hydroxy-3-methyl-benzaldehyde (1 g, 7.34 mmol, 1 eq) in CH₃NO₂ (10 mL) was added NH₄OAc (113.23 mg, 1.47 mmol, 0.2 eq). The mixture was stirred at 110° C. for 2 h. LC-MS showed 4-hydroxy-3-methyl-benzaldehyde was consumed completely and one main peak with desired m/z was detected. The reaction mixture was concentrated under reduced pressure. The residue was purified by column chromatography (SiO₂, Petroleum ether/Ethyl acetate=80/1 to 0/1). Compound 2-methyl-4-[(E) nitrovinyl]phenol (700 mg, 3.91 mmol, 53.19% yield) was obtained as yellow solid.

Step 2:

To a mixture of LiAlH₄ (105.92 mg, 2.79 mmol, 10 eq) in THF (10 mL) was added 2-methyl-4-[(E)-2-nitrovinyl]phenol (50 mg, 279.06 umol, 1 eq) in THF (5 mL) at 0° C. under N₂. The mixture was stirred at 0° C. for 2 h, and then the mixture was stirred at 70° C. for 12 h. LC-MS showed 2-methyl-4-[(E)-2-nitrovinyl]phenol was consumed completely. The suspension was cooled to 0° C. and the excess of LiAlH₄ was quenched with 6 M aqueous sodium hydroxide (1 mL). The precipitate was filtered off and the filter cake was washed with EtOAc (5 mL). The combined organic layers were washed with brine and dried Na₂SO₄. The residue was purified by prep-HPLC (column: Nano-micro Kromasil C18 80*25 mm 3 μm; mobile phase: [water (0.04% HCl)-ACN]; B %: 5%-25%, 7 min). Compound 4-(2-aminoethyl)-2-methyl-phenol (3 mg, 15.83 μmol, 2.39% yield, 99% purity, HCl) was obtained as a white solid. LC-MS m/z=152.0. ¹H NMR (400 MHz, CD₃OD) δ 6.97 (s, 1H), 6.90 (d, J=8.2 Hz, 1H), 6.71 (d, J=8.2 Hz, 1H), 3.10 (t, J=7.6 Hz, 2H), 2.81 (t, J=7.6 Hz, 2H), 2.18 (s, 3H).

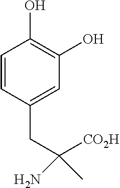

### Compound Q: 2-amino-3-(3,4-dihydroxyphenyl)-2-methylpropanoic acid

Step 1:

To a mixture of tert-butyl 2-(benzhydrylideneamino)acetate (2 g, 6.77 mmol, 1 eq) in DMF (20 mL) was added NaH (324.98 mg, 8.13 mmol, 60% purity, 1.2 eq) in one portion at 0° C. The mixture was stirred at 0° C. for 0.5 h. Then to the mixture was added 4-(bromomethyl)-1,2-dimethoxy-benzene (1.88 g, 8.13 mmol, 1.2 eq). The mixture was stirred at 25° C. for 2 h. LC-MS showed Reactant was consumed completely and one main peak with desired m/z (M+1=446.2, RT=2.434 min) was detected. The mixture was poured to sat. NaHCO₃ (40 mL) at 0-5° C. The mixture was extracted with ethyl acetate (20 mL*3). The combined organic phase was washed with brine (15 mL*4), dried with anhydrous Na₂SO₄, filtered and concentrated in vacuum. The residue was purified by column chromatography (SiO₂, Petroleum ether/Ethyl acetate=20/1 to 3/1). Compound tert-butyl 2-(benzhydrylideneamino)-3-(3,4-dimethoxyphenyl)propanoate (1.9 g, 4.26 mmol, 62.98% yield) was obtained as a yellow oil.

Step 2:

To the mixture LDA (2 M, 3.93 mL, 7 eq) in THF (6 mL) was added the solution tert-butyl 2-(benzhydrylideneamino)-3-(3,4-dimethoxyphenyl)propanoate (0.5 g, 1.12 mmol, 1 eq) in HMPA (1.86 g, 10.39 mmol, 1.83 mL, 9.26 eq) and THF (3 mL) at −70° C. under N₂. The mixture was stirred at −70° C. for 0.5 h. Then to the mixture was added Mel (1.59 g, 11.22 mmol, 698.62 μL, 10 eq) drop-wise at −70° C. The mixture was allowed to warm to 25° C. and stirred at 25° C. for 1 h. LC-MS indicated Reactant was consumed completely. The mixture was poured into sat. NaHCO₃ (15 mL) and extracted with ethyl acetate (15 mL*3), dried with anhydrous Na₂SO₄, filtered and concentrated in vacuum. The residue was purified by column chromatography (SiO₂, Petroleum ether/Ethyl acetate=20/1 to 8/1). Compound tert-butyl 2-(benzhydrylideneamino)-3-(3,4-dimethoxyphenyl)-2-methyl-propanoate (0.32 g, 696.30 μmol, 62.05% yield) was obtained as a yellow oil.

Step 3:

The mixture of tert-butyl 2-(benzhydrylideneamino)-3-(3,4-dimethoxyphenyl)-2-methyl-propanoate (0.27 g, 587.50 μmol, 1 eq) in aq. HBr (8.32 g, 41.12 mmol, 5.58 mL, 40% purity, 70 eq) was stirred at 100° C. for 4 h. TLC (Petroleum ether: Ethyl acetate=10:1) indicated rt-butyl 2-(benzhydrylideneamino)-3-(3,4-dimethoxyphenyl)-2-methyl-propanoate was consumed completely. The reaction mixture was extracted with EtOAc (15 mL*3). The aqueous layer was concentrated under reduced pressure to remove the organic. The crude was purified by prep-HPLC (column: Nano-micro Kromasil C18 80*25 mm 3 μm; mobile phase: [water (0.04% HCl)-ACN]; B %: 1%-8%, 7 min). The crude product was further purified by prep-HPLC (column: Welch Xtimate C18 150*25 mm*5 μm; mobile phase: [water (0.04% HCl)-ACN]; B %: 1%-10%, 10 min). 2-amino-3-(3,4-dihydroxyphenyl)-2-methyl-propanoic acid (15 mg, 70.31 umol, 12% yield, 99% purity) was obtained as white solid as HCl salt. LC-MS m/z=212.1. ¹H NMR (400 MHz, CD₃OD) δ 6.76 (d, J=8.4 Hz, 1H), 6.71 (s, 1H), 6.59 (d, J=8.4 Hz, 1H), 3.19 (d, J=14.2 Hz, 1H), 2.93 (d, J=14.2 Hz, 1H), 1.61 (s, 3H).

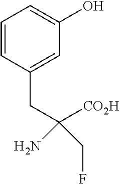 [SMILES: NC(CF)(Cc1cccc(O)c1)C(=O)O]

### Compound R: 2-amino-3-fluoro-2-(3-hydroxybenzyl)propanoic acid

Step 1:

To the solution of 2-amino-3-(3-hydroxyphenyl)propanoic acid (10 g, 55.19 mmol, 1 eq) in tert-butyl acetate (86.60 g, 745.54 mmol, 100.00 mL, 13.51 eq) was added perchloric acid (12.67 g, 88.31 mmol, 7.63 mL, 70% purity, 1.6 eq) drop-wise at 0° C. The mixture was stirred at 25° C. for 10 h. TLC (Dichloromethane:Methanol=10:1, R_{f}=0.30) showed ˜20% of R2-amino-3-(3-hydroxyphenyl)propanoic acid remained. One new spot was shown on TLC. EtOAc (50 mL) was added to the mixture, then the mixture was washed with H₂O (50 mL). Then the organic phase was extracted with 1N HCl (10 mL). The combined aqueous phase was adjusted to pH=9 by 10% K₂CO₃ solution. Then the aqueous phase was extracted with DCM (30 mL*3). The combined organic phase was washed with brine (20 mL), dried with anhydrous Na₂SO₄, filtered and concentrated in vacuum. Tert-butyl 2-amino-3-(3-hydroxyphenyl)propanoate (4.35 g, 18.33 mmol, 33.21% yield) was obtained as off-white solid.

Step 2:

To the solution of tert-butyl 2-amino-3-(3-hydroxyphenyl)propanoate (4.35 g, 18.33 mmol, 1 eq) in toluene (90 mL) was added 4A molecular sieve (4.35 g) and TsOH (157.84 mg, 916.58 μmol, 0.05 eq). The mixture was stirred at 25° C. for 30 min under N₂. To the mixture was added diphenylmethanone (3.67 g, 20.16 mmol, 1.1 eq). The mixture was stirred at 110° C. for 9.5 h. TLC (Petroleum ether: Ethyl acetate=5:1, R_{f}=0.50) indicated tert-butyl 2-amino-3-(3-hydroxyphenyl)propanoate was consumed completely. The reaction mixture was cooled to 25° C. Then the mixture was filtered. The filter cake was washed with EtOAc (50 mL*2). The combined organic phase was concentrated in vacuum. The residue was purified by flash silica gel chromatography (ISCO®; 100 g SepaFlash® Silica Flash Column, Eluent of 0-15% Ethyl acetate/Petroleum ethergradient @ 80 mL/min). Tert-butyl 2-(benzhydrylideneamino)-3-(3-hydroxyphenyl)propanoate (2.4 g, 5.98 mmol, 32.61% yield) was obtained as yellow solid.

Step 3:

To the mixture of tert-butyl 2-(benzhydrylideneamino)-3-(3-hydroxyphenyl)propanoate (2.4 g, 5.98 mmol, 1 eq) in DMF (25 mL) was added NaH (286.90 mg, 7.17 mmol, 60% purity, 1.2 eq) at 0° C. The mixture was stirred at 15° C. for 30 min. To the mixture was added MOMCI (673.79 mg, 8.37 mmol, 635.65 μL, 1.4 eq) drop-wise at 0° C. The mixture was stirred at 25° C. for 2 h. TLC (Petroleum ether: Ethyl acetate=5:1, R_{f}=0.65) indicated tert-butyl 2-(benzhydrylideneamino)-3-(3-hydroxyphenyl)propanoate was consumed completely. The mixture was added slowly to saturated aq. NaHCO₃ (75 mL). The aqueous phase was extracted with MTBE (30 mL*3). The combined organic phase was washed with brine (15 mL*3), dried with anhydrous Na₂SO₄, filtered and concentrated in vacuum. tert-butyl 2-(benzhydrylideneamino)-3-[3-(methoxymethoxy)phenyl]propanoate (2.28 g, 5.12 mmol, 85.61% yield) was obtained as yellow oil.

Step 4:

To the solution of THF (30 mL) was added LDA (2 M, 17.91 mL, 7 eq) under N₂ then cooled to −70° C. To the mixture was added tert-butyl 2-(benzhydrylideneamino)-3-[3-(methoxymethoxy) phenyl]propanoate (2.28 g, 5.12 mmol, 1 eq) in HMPA (8.49 g, 47.39 mmol, 8.33 mL, 9.26 eq) and THF (20 mL) drop-wise at −70° C. The mixture was stirred at −70° C. for 0.5 h. Then fluoro(iodo)methane (8.18 g, 51.17 mmol, 10 eq) was added drop-wise at −70° C. The mixture was stirred at 25° C. for 1 h. TLC (Petroleum ether: Ethyl acetate=10:1, R_{f}=0.66) showed the starting material was consumed completely. The mixture was poured into aq. NaHCO₃ (50 mL) slowly at 0-5° C. The mixture was extracted with ethyl acetate (10 mL*3). The combined organic phase was washed with brine (5 mL*2), dried with anhydrous Na₂SO₄, filtered and concentrated in vacuum. The residue was purified by flash silica gel chromatography (ISCO®; 10 g SepaFlash® Silica Flash Column, Eluent of 0-5% Ethyl acetate/Petroleum ethergradient @ 40 mL/min). tert-butyl 2-(benzhydrylideneamino) (fluoromethyl)-3-[3-(methoxymethoxy)phenyl]propanoate (1.38 g, 2.89 mmol, 56.47% yield) was obtained as yellow oil.

Step 5:

To the solution of tert-butyl 2-(benzhydrylideneamino)-2-(fluoromethyl)-3-[3-(methoxymethoxy) phenyl]propanoate (1.38 g, 2.89 mmol, 1 eq) in THF (35 mL) was added citric acid (28.98 g, 7.54 mmol, 29.01 mL, 5% purity, 2.61 eq). The mixture was stirred at 25° C. for 6 h. TLC (Petroleum ether: Ethyl acetate=10:1, R_{f}=0.13) indicated tert-butyl 2-(benzhydrylideneamino)-2-(fluoromethyl)-3-[3-(methoxymethoxy)phenyl]propanoate was consumed completely. One new spot with large polarity was detected. The mixture was concentrated in reduced pressure to remove THF. The aqueous phase was extracted with ethyl acetate (10 mL*3). The combined organic phase was washed with brine (10 mL), dried with anhydrous Na₂SO₄, filtered and concentrated in vacuum. The residue was purified by column chromatography (SiO₂, Petroleum ether/Ethyl acetate=10/1 to 0/1). tert-butyl 2-amino-2-(fluoromethyl)-3-[3-(methoxymethoxy)phenyl]propanoate (460 mg, 1.35 mmol, 46.74% yield, 92% purity) was obtained as white solid.

Step 6:

To the mixture of tert-butyl 2-amino-2-(fluoromethyl)-3-[3-(methoxymethoxy)phenyl]propanoate (240 mg, 765.88 umol, 1 eq) in THF (20 mL) was added NaHCO₃ (64.34 mg, 765.88 umol, 29.79 μL, 1 eq) in H₂O (10 mL). The mixture was cooled to 0° C. To the mixture was added CbzCl (156.78 mg, 919.06 μmol, 130.65 μL, 1.2 eq) slowly at 0° C. The mixture was stirred at 25° C. for 2 h. TLC (Petroleum ether: Ethyl acetate=5:1, Rf=0.60) indicated Reactant was consumed completely. Two same scale batches were combined together for work-up and purification. The mixture was extracted with ethyl acetate (10 mL*4). The combined organic phase was washed with brine (10 mL), dried with anhydrous Na₂SO₄, filtered and concentrated in vacuum. The residue was purified by prep-TLC (SiO₂, Petroleum ether: Ethyl acetate=5:1). tert-butyl 2-(benzyloxycarbonylamino)-2-(fluoromethyl)- 3-[3-(methoxymethoxy)phenyl]propanoate (400 mg, 893.86 μmol, 58.36% yield) was obtained as light yellow oil.

Step 7:

To the mixture of tert-butyl 2-(benzyloxycarbonylamino)-2-(fluoromethyl)-3-[3-(methoxymethoxy)phenyl]propanoate (300 mg, 670.40 μmol, 1 eq) in THF (10 mL) was added aq. HCl (2.5 M, 5.36 mL, 20 eq). The mixture was stirred at 60° C. for 3 h. LC-MS showed tert-butyl 2-(benzyloxycarbonylamino)-2-(fluoromethyl)-3-[3-(methoxymethoxy) phenyl]propanoate was consumed completely. The mixture was concentrated in reduced pressure to remove THF. The aqueous phase was extracted with ethyl acetate (5 mL*3). The combined organic phase was washed with brine (3 mL), dried with anhydrous Na₂SO₄, filtered and concentrated in vacuum. The product was purified by prep-HPLC (column: Welch Xtimate C18 150*30 mm*5 μm; mobile phase: [water (10 mM NH₄HCO₃)-ACN]; B %: 45%-75%, 3 min). tert-butyl 2-(benzyloxycarbonylamino)-2-(fluoromethyl)-3-(3-hydroxyphenyl)propanoate (157 mg, 389.15 μmol, 58.05% yield) was obtained as colorless oil.

Step 8:

To the mixture of tert-butyl 2-(benzyloxycarbonylamino)-2-(fluoromethyl)-3-(3-hydroxyphenyl) propanoate (100 mg, 247.87 umol, 1 eq) in ACN (20 mL) was added TMSI (148.79 mg, 743.60 μmol, 101.22 μL, 3 eq). The mixture was stirred at 25° C. for 2 h. TLC (Petroleum ether: Ethyl acetate=5:1, R_{f}=0.02) indicated the starting material was consumed completely. The mixture was concentrated in reduced pressure. The crude was purified by prep-HPLC (column: Welch Xtimate C18 150*25 mm*5 μm; mobile phase: [water (0.04% HCl)-ACN]; B %: 1%-3%, 10 min). 2-amino-2-(fluoromethyl)-3-(3-hydroxyphenyl)propanoic acid (27 mg, 96.36 μmol, 38.87% yield, 89.1% purity, HCl) was obtained as yellow solid. LC-MS m/z=214.1. ¹H NMR (400 MHz, CD₃OD) δ 7.19 (app t, J=8.2 Hz, 1H), 6.78 (d, J=8.2 Hz, 1H), 6.71 (d, J=8.2 Hz, 1H), 6.69 (s 1H), 4.96 (dd, J=46, 10.4 Hz, 1H), 4.69 (dd, J=46, 10.4 Hz, 1H), 3.29 (d, J=14.4 Hz, 1H), 3.05 (d, J=14.4 Hz, 1H).

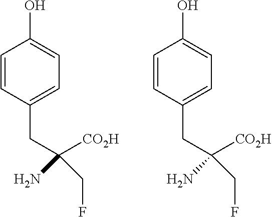 [SMILES: N[C@@](CF)(Cc1ccc(O)cc1)C(=O)O ; N[C@](CF)(Cc1ccc(O)cc1)C(=O)O]

### Compounds S and T: (S)-2-amino-3-fluoro-2-(4-hydroxybenzyl)propanoic acid and (R)-2-amino-3-fluoro-2-(4-hydroxybenzyl)propanoic acid

Step 1:

Two reactions were carried out in parallel and combined together for work-up. To a solution of tert-butyl L-tyrosinate (60 g, 253 mmol, 1.00 eq) in dry DCM (300 mL) were added TsOH (6.53 g, 37.9 mmol, 0.15 eq) and MgSO₄ (60.9 g, 505 mmol, 2.00 eq) in one portion. After the addition, the suspension was stirred at 25° C. for 0.5 h. Benzaldehyde (29.5 g, 278 mmol, 28.1 mL, 1.10 eq) was added to the solution in one portion. After addition, the suspension was stirred at 25° C. for 12 h. H NMR (ET27430-13-P1A1) showed the starting material was consumed completely. Two reactions were combined together for work-up. The suspension was filtered, and the filter cake was washed by DCM (200 mL×2). The filtrate was washed by cold aqueous solution of NaHCO₃ (Sat. 800 mL). The organic phase was dried over Na₂SO₄, and concentrated under vacuum to give a solid. The solid was dried in the air for 12 h to oxidize PhCHO into PhCOOH. The solid was triturated with MTBE/Petroleum ether (2/1) at 25° C. for 30 min. The suspension was filtered, and the filter cake was dissolved in DCM (300 mL). The organic layer was washed with cold aqueous solution of NaHCO₃ (Sat. 200 mL). The organic phase was dried over Na₂SO₄, filtered and concentrated under vacuum to give tert-butyl (S,E)-2-(benzylideneamino)-3-(4-hydroxyphenyl)propanoate (110 g, 338 mmol, 66.9% yield) as an off-white solid.

Step 2:

Four reactions were carried out in parallel and combined together for work-up. To a solution of tert-butyl (S,E)-2-(benzylideneamino)-3-(4-hydroxyphenyl)propanoate (20 g, 61.5 mmol, 1.00 eq) in DMF (200 mL) was added NaH (2.70 g, 67.6 mmol, 60% purity, 1.10 eq) at 0° C. in portions. After the addition, the resulting suspension was stirred at 0° C. for 1.5 h. MOMCI (4.95 g, 61.5 mmol, 4.67 mL, 1.00 eq) was added to the suspension in one portion, and the suspension was stirred at 0° C. for 1 h. LC-MS (ET27430-22-P1A2) showed the starting material was consumed completely and one main peak with desired mass was detected. Four reactions were combined together for work-up. The suspension was slowly poured into cold saturated aqueous solution of NaHCO₃ (1200 mL) and extracted with MTBE (300 mL×4). The combined organic layer was washed with cold brine (600 mL×2), dried over Na₂SO₄, filtered and concentrated under reduced pressure to give tert-butyl (S,E)-2-(benzylideneamino)-3-(4-(methoxymethoxy)phenyl)propanoate (90.3 g, crude) as a yellow oil which was used in the next step directly.

Step 3:

Four reactions were carried out in parallel and combined together for work-up. LDA (2 M, 29.8 mL, 2.20 eq) was added to THF (50 mL) at −70° C. drop-wise. After the addition, HMPA (12.1 g, 67.7 mmol, 11.9 mL, 2.50 eq) was added to the solution in one portion, and followed by a solution of tert-butyl (S,E)-2-(benzylideneamino)-3-(4-(methoxymethoxy)phenyl)propanoate (10.0 g, 27.1 mmol, 1.00 eq) in THF (20 mL) drop-wise at −70° C. The solution was stirred at −70° C. for 1 h. Fluoroiodomethane (10.8 g, 67.7 mmol, 2.50 eq) was added to the solution drop-wise at −70° C. After the addition, the solution was stirred at −70° C. for 1 h. LC-MS (ET27430-25-P1A2) showed the starting material was consumed completely and one main peak with desired mass was detected. Four reactions were combined together for work-up. The solution was slowly poured into cold aqueous solution of NaHCO₃(Sat. 600 mL) and extracted with MTBE (300 mL×4). The combined organic layer was dried over Na₂SO₄, filtered and concentrated under vacuum to give tert-butyl (E)-2-(benzylideneamino)-3-fluoro-2-(4-(methoxymethoxy)benzyl)propanoate (50 g, crude) as a brown oil which was used in the next step directly.

Step 4:

To a solution of tert-butyl (E)-2-(benzylideneamino)-3-fluoro-2-(4-(methoxymethoxy)benzyl)propanoate (60 g, 149 mmol, 1.00 eq) in THF (150 mL) was added citric acid monohydrate (1.08 kg, 257 mmol, 1200 mL, 5% purity, 1.72 eq). The solution was stirred at 20° C. for 5 h. TLC (Petroleum ether: Ethyl acetate=10:1, R_{f} of material=0.5) showed the starting material was consumed completely, and one major new spot with higher polarity was detected. LC-MS (ET27430-27-P1A) showed the reaction was completed. The solution was extracted with MTBE: Petroleum ether=1:1 (500 mL×2). The organic layer was washed by water (500 mL), and the organic layer was discarded. The combined aqueous layer was poured into aqueous solution of NaHCO₃ (Sat. 600 mL). The aqueous phase was extracted with MTBE (600 mL×3). The combined organic layers were dried over Na₂SO₄, filtered and concentrated under reduced pressure to give a residue. The residue was purified by column chromatography (SiO₂, NH₃.H₂O/Petroleum ether/Dichloromethane=0/4/1, 0/2/1, 0.005/2/1, 0.005/1/1, 0.005/0/1) to give tert-butyl 2-amino-3-fluoro-2-(4-(methoxymethoxy)benzyl)propanoate (18 g, 57.44 mmol, 38.43% yield) as a brown oil. The racemic mixture (18 g, 57.44 mmol) was purified by SFC (column: DAICEL CHIRALPAK AD-H (250 mm*30 mm, 5 μm); mobile phase: [0.1% NH₃.H₂O ETOH]; B %: 15%-15%, 2.3 min). (S)tert-butyl 2-amino-3-fluoro-2-(4-(methoxymethoxy)benzyl)propanoate (7.4 g, 23.6 mmol, 92.5% yield) was obtained as a brown oil and (R)-tert-butyl 2-amino-3-fluoro-2-(4-(methoxymethoxy)benzyl)propanoate (7.1 g, 22.7 mmol, 88.8% yield) was obtained as a brown oil.

Step 5:

To a solution of (S)tert-butyl 2-amino-3-fluoro-2-(4-(methoxymethoxy)benzyl)propanoate (6.52 g, 20.8 mmol, 1.00 eq) in THF (30 mL) was added aqueous solution of HCl (2.5 M, 83.2 mL, 10.0 eq). The solution was stirred at 60° C. for 3 h. LC-MS (ET27430-31-P1A2) showed the reaction was completed. The reaction solution was lyophilized to give a crude product. The crude product was purified by pre-HPLC (column: Phenomenex luna C18 250*80 mm*10 μm; mobile phase: [water (0.05% HCl)-ACN]; B %: 0%-9%, 20 min) to give (S)-2-amino-3-fluoro-2-(4-hydroxybenzyl)propanoic acid (2.3 g, 10.8 mmol, 51.9% yield) as a white solid. LC-MS m/z=214. ¹H NMR (400 MHz, DMSO-d6) δ 8.68 (br s, 1H), 7.01 (d, J=8.4 Hz, 1H), 6.72 (d, J=8.4 Hz, 1H), 4.84 (dd, J=45.2, 10.0 Hz, 1H), 4.63 (d, J=45.2, 10.0 Hz, 1H), 3.04 (d, J=14.0 Hz, 1H), 2.96 (d, J=14.0 Hz, 1H).

To a solution of (R)-tert-butyl 2-amino-3-fluoro-2-(4-(methoxymethoxy)benzyl)-propanoate (6.77 g, 21.6 mmol, 1.00 eq) in THF (30 mL) was added aqueous solution of HCl (2.5 M, 86.4 mL, 10.0 eq). The solution was stirred at 60° C. for 3 h. LC-MS showed the reaction was completed. The reaction solution was lyophilized to give a crude product. The crude product was purified by pre-HPLC (column: Phenomenex luna C18 250*80 mm*10 μm; mobile phase: [water (0.05% HCl)-ACN]; B %: 0%-9%, 20 min) to give (R)-2-amino-3-fluoro-2-(4-hydroxybenzyl)propanoic acid (2.3 g, 10.8 mmol, 49.9% yield) as a white solid. ¹H NMR (400 MHz, DMSO-d6) δ 8.68 (br s, 1H), 7.01 (d, J=8.4 Hz, 1H), 6.72 (d, J=8.4 Hz, 1H), 4.84 (dd, J=45.2, 10.0 Hz, 1H), 4.63 (d, J=45.2, 10.0 Hz, 1H), 3.04 (d, J=14.0 Hz, 1H), 2.96 (d, J=14.0 Hz, 1H).

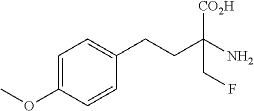 [SMILES: COc1ccc(CCC(N)(CF)C(=O)O)cc1]

### Compound U: 2-amino-2-(fluoromethyl)-4-(4-methoxyphenyl)butanoic acid

To a solution of 3-(4-methoxyphenyl)propanal in a 250-mL round-bottomed flask was charged with NH₄Cl (1.2 eq), ammonia (3 eq), ethanol (0.2 M), and water (0.2 M). The mixture was dissolved into a clear solution. NaCN (1.5 eq) was added to the mixture. The flask was sealed quickly with a rubber stopper. The mixture was stirred for 3 days and extracted with CH₂Cl₂ (100 mL). The combined organic layer was washed with water to remove the remaining NaCN. The mixture was dried with anhydrous sodium sulfate. The mixture was concentrated under reduced pressure to afford the product. The residue was purified by column chromatography to give 2-amino-4-(4-methoxyphenyl)butanenitrile in 70-80%. Yield. A mixture of the 2-amino-4-(4-methoxyphenyl)butanenitrile, Et₃N and benzophenone(1:1.3:8 molar ratio, respectively) and DMF (7 mL/g ketone) was loaded in a round-bottomed, two-necked flask fitted with a refluxing condenser. A toluene (1 M) solution of TiCl₄, (0.9 molar with respect to the substrate) was carefully added dropwise to the solution. After the addition was completed, the mixture was refluxed (35-40° C.) for 1 h and then allowed to stand 6 h at room temperature. The suspension was concentrated and extracted by diethyl ether and purified by column chromatography to give 2-((diphenylmethylene)amino)-4-(4-methoxyphenyl)butanenitrile in 23-30%. Yield. 2-amino-4-(4-methoxyphenyl)butanenitrile was treated with 11 eq of HBr (48 wt. % in H₂O) and the solution was heated to 60° C. for five days. Progress of the reaction was monitored by LC-MS. The crude compound was purified by reverse phase column chromatography to give 2-amino-2-(fluoromethyl)-4-(4-hydroxyphenyl)butanoic acid 2-amino-2-(fluoromethyl)-4-(4-hydroxyphenyl)butanoic acid in 20% yield. ¹H NMR (500 MHz, Deuterium Oxide) δ 7.34-7.26 (m, 2H), 7.02-6.96 (m, 2H), 5.11-4.76 (m, 2H), 2.89-2.78 (m, 1H), 2.70 (td, J=12.9, 5.0 Hz, 1H), 2.34-2.12 (m, 1H). LC-MS: (M+1) 228.3, (M-1) 226.2.

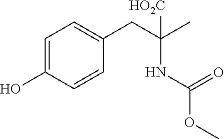 [SMILES: COC(=O)NC(C)(Cc1ccc(O)cc1)C(=O)O]

### Compound V: 3-(4-hydroxyphenyl)-2-((methoxycarbonyl)amino)-2-methylpropanoic acid

Methyl chloroformate (1 eq) was added to a solution of 2-amino-3-(4-hydroxyphenyl)-2-methylpropanoic acid and NaHCO₃ (20 eq) in a mixture of H₂O/THF (2 M). The mixture was stirred at room temperature overnight and then diluted with H₂O. The mixture was washed with Et₂O. The aqueous layer was acidified to pH ˜2-3, evaporated to dryness, and purified by reverse phase column chromatography to give 3-(4-hydroxyphenyl) ((methoxycarbonyl)amino)-2-methylpropanoic acid with 70% yield. ¹H NMR (400 MHz, Methanol-d₄) δ 7.02-6.89 (m, 2H), 6.77-6.59 (m, 2H), 3.64 (s, 3H), 3.21-2.96 (m, 2H), 1.41 (s, 3H). LC-MS: (M+1): 254.2, (M-1): 252.2.

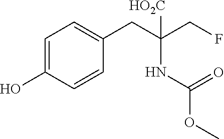 [SMILES: COC(=O)NC(CF)(Cc1ccc(O)cc1)C(=O)O]

### Compound W: 3-fluoro-2-(4-hydroxybenzyl)-2-((methoxycarbonyl)amino)-propanoic acid

Methyl chloroformate (1 eq) was added to a solution of (S)-2-amino-3-fluoro-2-(4-hydroxybenzyl)propanoic acid and NaHCO₃ (20 eq) in a mixture of H₂O/THF (2 M). The mixture was stirred at room temperature overnight and diluted with H₂O. The mixture was washed with Et₂O. The aqueous layer was acidified to pH ˜2-3, evaporated to dryness, and purified by reverse phase column chromatography to give 3-fluoro-2-(4-hydroxybenzyl)-2-((methoxycarbonyl)amino)propanoic acid with 73% yield. ¹H NMR (400 MHz, Methanol-d₄) δ 6.95 (d, J=8.5 Hz, 2H), 6.79-6.59 (m, 2H), 4.70 (dt, J=47.2, 8.8 Hz, 2H), 3.66 (s, 3H), 3.15-2.93 (m, 2H). LC-MS: (M+1): 272.2, (M-1): 270.1.

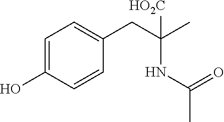 [SMILES: CC(=O)NC(C)(Cc1ccc(O)cc1)C(=O)O]

### Compound X: 2-acetamido-3-(4-hydroxyphenyl)-2-methylpropanoic acid

To a solution of 2-amino-3-(4-hydroxyphenyl)-2-methylpropanoic acid and Hunig's base (5 eq) in THF (0.2 M) was added acetic anhydride (1.5 eq). The mixture was stirred at room temperature overnight and diluted with H₂O. The mixture was washed with Et₂O. The aqueous layer was acidified to pH ˜2-3, evaporated to dryness, and purified by reverse phase column chromatography to give 2-acetamido-3-(4-hydroxyphenyl)-2-methylpropanoic acid with 75% yield. ¹H NMR (400 MHz, Methanol-d₄) δ 6.93 (d, J=8.4 Hz, 2H), 6.68 (d, J=8.5 Hz, 2H), 3.26 (d, J=13.6 Hz, 2H), 3.03 (d, J=13.6 Hz, 1H), 1.92 (s, 3H), 1.37 (s, 3H). LC-MS: (M+1): 238.2, (M-1): 236.2.

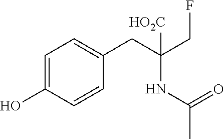 [SMILES: CC(=O)NC(CF)(Cc1ccc(O)cc1)C(=O)O]

### Compound Y: 3-fluoro-2-(4-hydroxybenzyl)-2-((methoxycarbonyl)amino)propanoic acid

To a solution of (S)-2-amino-3-fluoro-2-(4-hydroxybenzyl)propanoic acid and Hunig's base (5 eq) in THF (0.2 M) was added acetic anhydride (1.5 eq). The mixture was stirred at room temperature overnight and diluted with H₂O. The mixture was washed with Et₂O. The aqueous layer was acidified to pH ˜2-3, evaporated to dryness, and purified by reverse phase column chromatography to give 2-acetamido-3-fluoro-2-(4-hydroxybenzyl)propanoic acid with 66% yield. ¹H NMR (400 MHz, Methanol-d₄) δ 7.06-6.90 (m, 2H), 6.75-6.62 (m, 2H), 4.79-4.70 (m, 1H), 4.68-4.57 (m, 2H), 3.08 (s, 2H), 1.98 (s, 3H). LC-MS: (M+Na): 278.2, (M-1): 254.2.

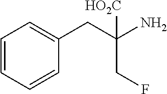 [SMILES: NC(CF)(Cc1ccccc1)C(=O)O]

### Compound Z: 2-amino-2-benzyl-3-fluoropropanoic acid

2-fluoroacetonitrile (1 eq) was dissolved in anhydrous Toluene (1 M) and cooled to 0° C. Phenylmagnesium chloride (2 M, 1 eq) was slowly added to the reaction mixture under an argon atmosphere. The reaction was stirred for 2 h and quenched by adding water (5 mL) and 1 M HCl (5 mL). 50 mL of ethyl acetate was added to this solution, and the organic layer was washed with water (2×20 mL), brine (2×20 mL) and dried over anhydrous sodium sulfate. The product was isolated by filtration and the solvent removed. The product was purified by column chromatography and carried to the next step directly. Ketone was transferred to a round-bottomed flask and charged with NH₄Cl (1.2 eq), ammonia (3 eq), ethanol (0.2 M), and water (0.2 M). The mixture was dissolved into a clear solution. NaCN (1.5 eq) was added to the mixture. The flask was sealed quickly with a rubber stopper. The mixture was stirred for 3 days and extracted with CH₂Cl₂ (100 mL). The combined organic layer was washed with water to remove the remaining NaCN. The mixture was dried with anhydrous sodium sulfate and concentrated under reduced pressure to afford the corresponding amino nitrile. The residue was purified by column chromatography and carried to the next step directly. The resulting nitrile was heated under reflux with a concentrated HCl in dioxane (0.4 M), such as dilute hydrochloric acid. A carboxylic acid formed and was evaporated to dryness and purified by reverse phase column chromatography to give 2-amino-2-benzyl-3-fluoropropanoic acid with 59% yield. ¹H NMR (400 MHz, DMSO-d6) δ 6.68-6.41 (m, 5H), 4.21 (d, J=10.3 Hz, 1H), 3.94 (dd, J=47.2, 10.3 Hz, 1H), 2.58-2.44 (m, 2H), 2.36 (d, J=14.2 Hz, 2H). LC-MS: (M+): 198.2.

### Example 2: Inhibition of Tyrosine Decarboxylase In Vitro

Tyrosine decarboxylase (tdc) was obtained by following a previously published literature procedure (Rekdal et al., Science 2019; 364(6445):eaau6323). The tdc (220 nM final concentration) was thawed on ice and then mixed with pyridoxal-5-phosphate (2.2 mM final concentration) in 200 mM sodium acetate buffer, pH 5.5 optionally containing 1 mM TCEP. To this mixture was added inhibitor at a final concentration of 1000, 333, 111, 37, 12, 4.1, 1.4, or 0 μM (final volume: 100 μL; inhibitor was 100-fold concentrated in a solution of DMSO, H₂O, or DMSO:H₂O (1/1 v/v)). The protein-inhibitor mixture was incubated at room temperature for 60 min. 6 μL of this mixture was then withdrawn from each solution and mixed with 54 μL of 10 mM levodopa in 200 mM sodium acetate buffer pH 5.5. The final concentration of the reaction was 22 nM tdc, 220 μM pyridoxal-5-phosphate, 9 mM levodopa in 200 mM sodium acetate buffer pH 5.5 with 0-100 μM inhibitor. The reaction proceeded for 5 min at room temperature before being quenched by the addition of 540 μL acetonitrile containing 0.1% (v/v) formic acid supplemented with 200 nM tolbutamide as an internal standard. The reactions were centrifuged (3,000 g, 10 min), and then 100 μL of each supernatant was transferred to a fresh plate. 100 μL of acetonitrile containing 0.1% (v/v) formic acid supplemented with 200 nM tolbutamide was added. An external standard curve containing 0-150 μM dopamine was prepared in the exact same manner.

Dopamine formed in each reaction was quantified by using an Agilent 6470 triple quadrupole mass spectrometer equipped with an Acquity UPLC. Mobile phase A consisted of H₂O containing 10 mM ammonium formate, pH 3.0 and supplemented with 0.1% (v/v) formic acid. Mobile phase B consisted of acetonitrile containing 10 mM ammonium formate, pH 3.0 and supplemented with 0.1% (v/v) formic acid. 5 μL of each sample was injected onto a BEH Amide column (Waters Corporation, 2.1×50 mm, 1.7 μm). The gradient was set to: 100% mobile phase B at 0 min, decreasing linearly to 65% mobile phase B by 1.5 min, held constant at 65% mobile phase B until 2.5 min, ramped back up to 100% mobile phase B by 2.6 min, and held constant at 100% mobile phase B until 4.2 min. The flow rate was 0.6 mL/min. The dopamine was detected by using the mass spectrometer in multiple reaction monitoring (MRM) mode, quantifying the transition 154.1 to 137.0 m/z in positive mode. The fragmentor setting was 74, the collision energy was 9, and the cell accelerator voltage was 4, and the dwell time was 20. Tolbutamide was monitored using MRM and quantifying the transition of 271.1 to 91.0 m/z in positive mode. The fragmentor setting was 88, the collision energy was 37, and the cell accelerator voltage was 4, and the dwell time was 20.

The amount of dopamine was quantified by normalizing the area to the area of tolbutamide internal standard within each sample. This relative response was then compared to that of the standard curve to obtain the dopamine formed within each sample. The concentration of dopamine formed as a function of the inhibitor concentration at the preincubation stage was plotted in GraphPad Prism 8, and the IC₅₀ was calculated using the non-linear fit for the standard IC₅₀ curve equation “[inhibitor] vs response (three parameters).”

TABLE 1

| | IC₅₀ @ 60 min |
|---|---|
| Compound | (μM) |
| U | >1000 |
| K | Minor inhibition |
| M | 3.2 |
| L | 2.5 |
| N | 2.6 |
| V | >1000 |
| W | >1000 |
| Y | >1000 |
| Q | >1000 |
| P | >1000 |
| X | >1000 |
| O | >1000 |
| R | Minor inhibition |
| Z | >1000 |
| T | 82.3 |
| S | 4 |

### Example 3: Inhibition of Enterococcus faecalis Decarboxylation Activity In Vitro

A vial of 200 μL of E. faecalis v583 was removed from a −80° C. freezer and thawed in an anaerobic chamber containing an atmosphere of either 95/5 N₂/H₂ (v/v) or 90/5/5 N₂/H₂/CO₂ (v/v). 200 μL was inoculated into 10 mL of sterile, anaerobic BHI broth, pH 5 (adjusted with NaOH). The culture was grown overnight at 37° C. under anaerobic conditions.

After overnight incubation, 40 μL of the saturated starter culture was mixed with 744 μL of sterile, anaerobic BHI broth, pH 5 that had been supplemented with 1.5 mM levodopa. To this was added 16 μL of a 50-fold concentrated stock solution of inhibitor that had been dissolved in either DMSO, H₂O, or DMSO:H₂O (1:1 v/v). The final concentration of the inhibitor in each condition was 0, 0.001, 0.01, 0.1, 1, 10, or 100 μM. The contents of each incubation were mixed, and then 100 μL was transferred into a fresh 96-well plate. A standard curve of levodopa (0-1.5 mM) in BHI broth, pH 5.5 was likewise prepared on a 100 μL scale and aliquoted into the plate. The plate was sealed and incubated for 24 h at 37° C. under an atmosphere of either 95/5 N₂/H₂ (v/v) or 90/5/5 N₂/H₂/CO₂ (v/v) in an anerobic chamber.

After 24 h incubation, the seal was removed, and the contents of each plate was mixed with 400 μL acetonitrile containing 0.1% (v/v) formic acid and 200 nM tolbutamide as an internal standard. The samples were mixed and then centrifuged (4,000 g, 10 min). 200 μL of each supernatant was transferred to a separate plate.

The samples were analyzed by using an Agilent 6470 triple quadrupole mass spectrometer equipped with an Acquity UPLC. Mobile phase A consisted of H₂O containing 10 mM ammonium formate, pH 3.0 and supplemented with 0.1% (v/v) formic acid. Mobile phase B consisted of acetonitrile containing 10 mM ammonium formate, pH 3.0 and supplemented with 0.1% (v/v) formic acid. 5 μL of each sample was injected onto a BEH Amide column (Waters Corporation, 2.1×50 mm, 1.7 μm). The gradient was set to: 100% mobile phase B at 0 min, decreasing linearly to 65% mobile phase B by 1.5 min, held constant at 65% mobile phase B until 2.5 min, ramped back up to 100% mobile phase B by 2.6 min, and held constant at 100% mobile phase B until 4.2 min. The flow rate was 0.6 mL/min. The levodopa was detected by using the mass spectrometer in multiple reaction monitoring (MRM) mode, quantifying the transition 198.1 to 151.9 m/z in positive mode. The fragmentor setting was 78, the collision energy was 13, and the cell accelerator voltage was 4, and the dwell time was 20. Tolbutamide was monitored using MRM and quantifying the transition of 271.1 to 91.0 m/z in positive mode. The fragmentor setting was 88, the collision energy was 37, and the cell accelerator voltage was 4, and the dwell time was 20.

The amount of levodopa was quantified by normalizing the area to the area of tolbutamide internal standard within each sample. This relative response was then compared to that of the standard curve to obtain the residual levodopa within each sample. The concentration of levodopa remaining as a function of inhibitor concentration was then plotted in GraphPad Prism 8, and the IC₅₀ was calculated using the non-linear fit for the standard IC₅₀ curve equation “[inhibitor] vs response (three parameters).”

TABLE 2

| | IC₅₀ @ 60 min |
|---|---|
| Compound | (μM) |
| V | >1000 |
| W | >1000 |
| Y | >1000 |
| Q | >1000 |
| P | >1000 |
| X | >1000 |
| O | >1000 |
| R | >1000 |
| Z | >1000 |
| T | 18.5 |
| S | 2 |

### Example 4: Inhibition of Dopamine Production in Fecal Matter

Fecal samples are assayed for the presence of the tvdc gene by attempting to amplify the gene with primers specific for it by qPCR. Samples that give a signal below the detection limit are used in subsequent steps.

E. faecalis v583 is grown as described in Example 3.

E. faecalis v583 is added to the samples at a dilution level calculated to represent 0, 0.1, 1, 2, 5, or 10% of the total organism present. The substrate (d₄-levodopa, 1 mM final concentration) is added to the mixture. An inhibitor of TyDC is also added at this time at a final concentration of 10 μM. Optionally, the IC₅₀ of an inhibitor is determined by adding an inhibitor across a range of appropriate concentrations, for example, 0, 0.001, 0.01, 0.1, 1, and 10 μM.

After incubation for a designated period of time and at a certain temperature (for example, 8 h at 37° C.), samples are rendered compatible with LC-MS analysis and the amount of product is determined using LC-MS analysis.

### Example 5: Preparation of Low-Volume Samples for Metabolomic Analysis

Plasma samples from healthy subjects and Parkinson's disease patients were obtained from BioIVT and kept at −80° C. until ready to use. A 100-μL aliquot of each sample (total volume of 0.5-1 mL) was transferred to a labeled Eppendorf tube placed on ice. The samples were diluted with 400 μL of crashing solution in LCMS-grade methanol containing the appropriate stable isotope-labeled internal standards. A blank sample was prepared by mixing 100 μL water with 400 μL of the crashing solution. Each tube was vortexed for 20 seconds and kept on ice for 10 min. The samples were subsequently centrifuged at 14,000 rpm for 20 min at 4° C. From the supernatant of each sample, two separate 200 μL aliquots were transferred into two separate Eppendorf tube labeled “RP” (reversed-phase) and “HILIC” (hydrophilic interaction liquid ion chromatography). All sample replicates were then concentrated under nitrogen flow, using Biotage TurboVap, gently with maximum pressure of 15 psi. The dried sample replicates were subsequently reconstituted with 40 μL of the corresponding reconstitution solutions—95% acetonitrile in water for HILIC or pure water for RP, both of which contain 10 μM each of d₄-tyramine and d₃-Levodopa as internal standards. Each tube was vortexed for at least 20 sec, then centrifuged at 14,800 rpm for 20 min at 4° C. A 30-μL portion of each reconstituted sample was transferred into a 300 mL glass insert inside the HPLC vial. A 1-μL portion of each replicate sample was analyzed by RP and HILIC, respectively.

### Example 6: Incubation of Meta-Tyramine with Hepatocytes

In a 50 mL falcon tube, 40 mL of Gibco hepatocyte thawing medium was added. The remaining 10 mL of medium was used to gently transfer hepatocytes into a falcon tube using a wide-mouth pipette. The mixture was centrifuged briefly at low speed (100 g for 30 sec) to gather cells at the base of the tube. The supernatant was removed from the tube then bleached and the liquid discarded, leaving behind the cell pellet in the tube. About 4 mL of Gibco Williams E phenol-free medium was added to the tube to get a final count of 1 mil/mL, tilting gently by hand to scatter the cell pellet. Cell count was confirmed by taking a small sample (approximately 100 nL), treating with blue dye, and counting live cells. Count was repeated twice and averaged to calculate the concentration in solution. Count of 1.2 mil/mL was found, and another 800 μL of incubation medium was added to the tube to bring the count to 1 mil/mL. Four wells of a 24 well plate were plated with 1 mL per well, and the plate was gently agitated to distribute a uniform number of cells per well. The plate was pre-incubated for about 10 min at 37° C. To begin incubation, 1 μL of meta-tyramine, para-tyramine, and Midazolam stock solutions were added into separate wells, and 5 μL of midazolam was added into a fourth well. The plates were agitated to distribute compounds, then 200 μL aliquots were taken from each well and treated with an equal volume of ice-cold acetonitrile to quench catalysis and monitor progress. The quenching and sampling procedure was repeated at 4 h for 4-h samples. The quenched samples were centrifuged at 4000 g for 10 min at 4° C. and the supernatants were saved for analysis.

### Example 7: In Vivo Metabolism of Levodopa

The microbial enzyme TDC in the rat microbiome was inhibited with alpha-fluoromethyltyrosine (AFMT). On day 0, male Sprague Dawley rats were prophylactically treated with single oral gavage of vehicle, carbidopa, carbidopa+S-AFMT, or carbidopa+R-AFMT, respectively. On day 1, solutions of levodopa, carbidopa, S-AFMT in 1% methyl cellulose (w/v), and 1% ascorbic acid (w/w) in deionized (DI) water were applied by oral gavage to male Sprague Dawley rats.

TABLE 3

| | | | Day: 0 | Day: 1 | |
|---|---|---|---|---|---|
| | | | Prophylactic | | Co-administered |
| | | | Inhibitor ONLY | Levodopa | Inhibitor |
| | # | Test | Concentration | Dose Level | Concentration |
| Group | Animals | Article | (mg/kg) | (mg/kg/dose) | (mg/kg) |
| 1 | 3 | Levodopa + | — | 50 | — |
| | | Vehicle | | | |
| 2 | 3 | Levodopa + | Carbidopa: 5 | 50 | Carbidopa: 5 |
| | | Carbidopa | | | |
| 3 | 3 | Levodopa + | Carbidopa: 5 | 50 | Carbidopa: 5 |
| | | Carbidopa + | S-AFMT: 50 | | I001: 50 |
| | | S-AFMT | | | |
| 4 | 3 | Levodopa + | Carbidopa: 5 | 50 | Carbidopa: 5 |
| | | Carbidopa + | R-AFMT: 50 | | I002: 50 |
| | | R-AFMT | | | |

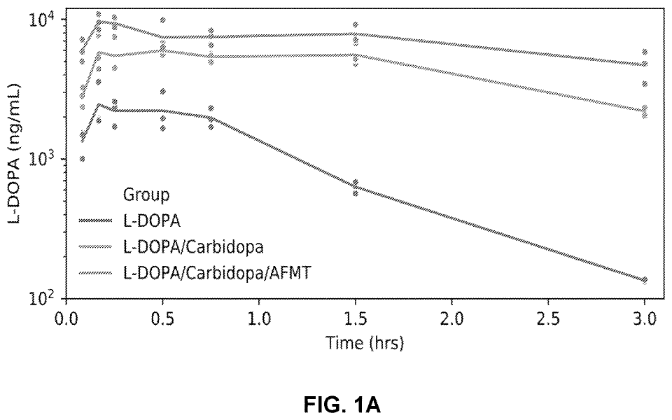

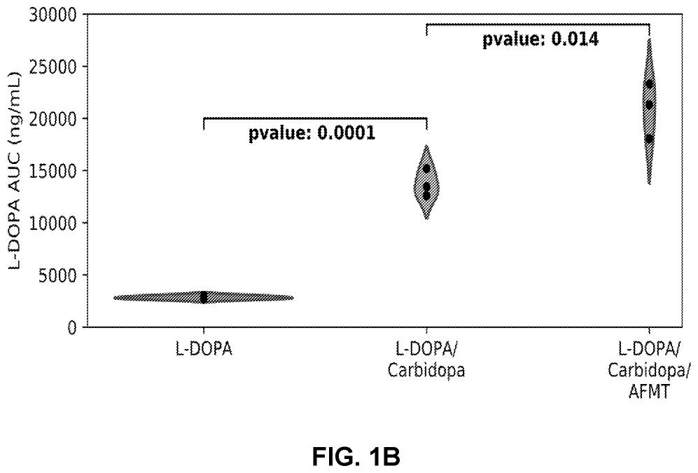

Whole blood samples were drawn and levodopa concentrations were determined by LC-MS/MS at 0, 5, 10, 15, 30, 45, 90, and 180 min post-application (12 animals per time point). Inhibition of TDC in the rat microbiome led to a 52% increase in levodopa in circulation over the first three hours, as well as elevated levels of levodopa at the earliest timepoint assessed (FIG. 1A-B).

### Example 8: Evaluating Pathways for In Vivo Metabolism of Levodopa

Using a high-resolution LC-MS metabolomics platform, commercially available human plasma samples from Parkinson's disease patients on levodopa therapy (PD) vs. healthy controls (HC) (18 vs. 18) were evaluated.

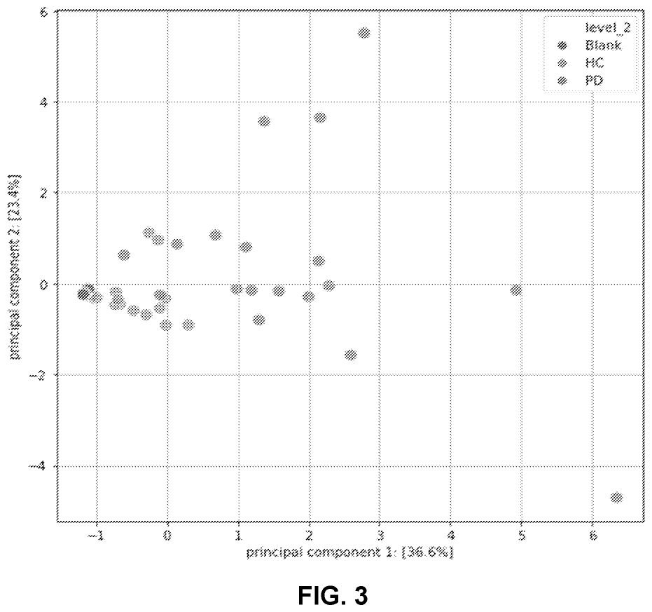

From the samples, a reaction pathway reflecting both microbe and human biotransformations and appearing to originate from microbial metabolism of levodopa to meta-tyramine was observed (FIG. 2A-D). Several downstream metabolites of meta-tyramine that maintain the uniquely-microbial 3-hydroxy group (e.g., 3-hydroxyphenylacetic acid, 3-hydroxyphenylacetate methyl ester, meta-tyramine-O-sulfate, and 3-sulfooxyphenylacetic acid) were specifically enriched in the PD cohort compared to HC, and collectively provided a discriminatory signal (FIG. 3).

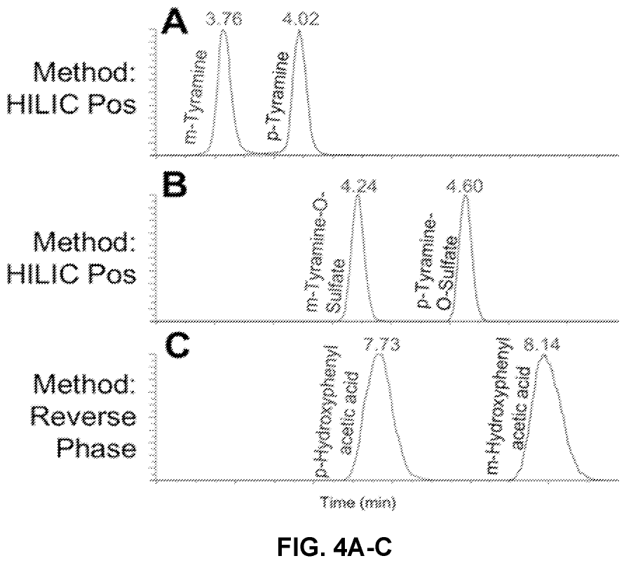

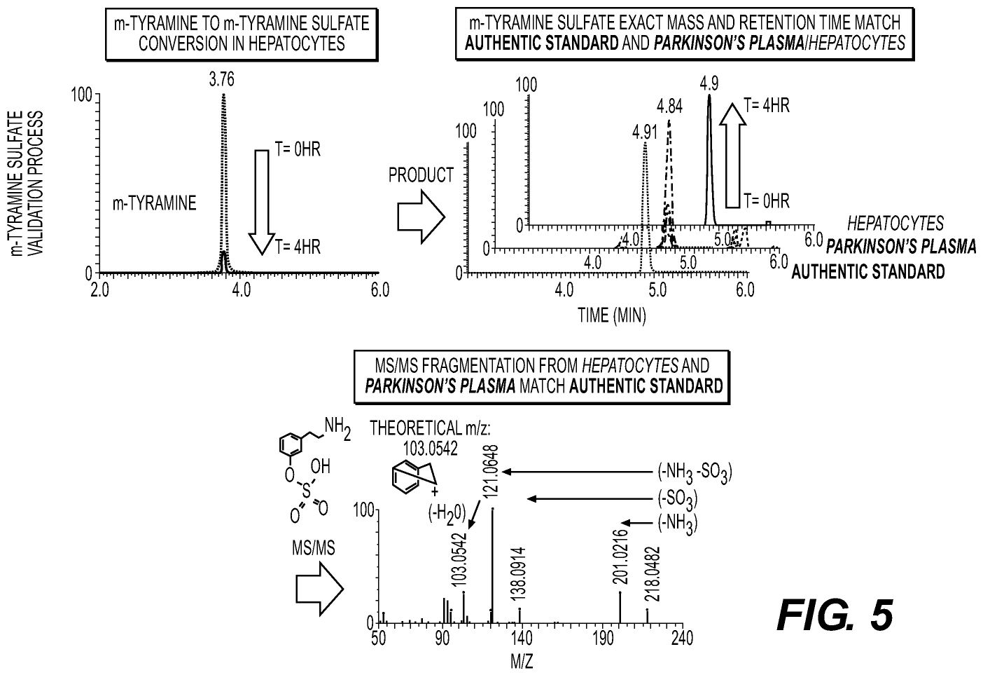

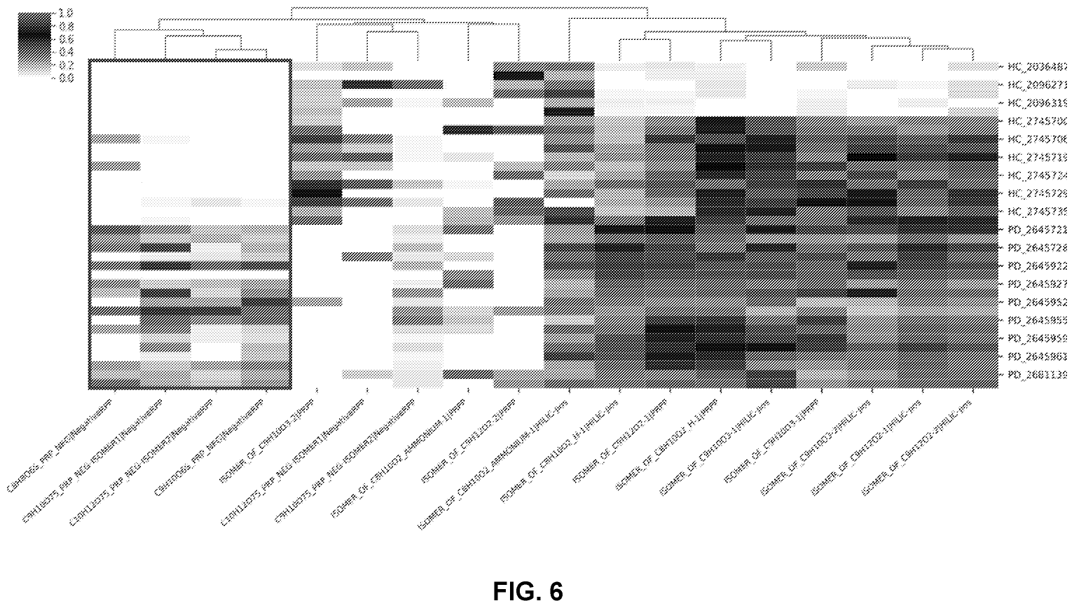

To validate that these downstream metabolites were produced through metabolism of meta-tyramine by the liver, hepatocytes were incubated with meta-tyramine as described in Example 6. Following a 4-h incubation, metabolic derivatives of meta-tyramine (e.g., 3-hydroxyphenylacetic acid and meta-tyramine-O-sulfate) were produced. Identity was confirmed by matching exact mass, retention time and MS/MS fragmentation with authentic standards (FIG. 4 and FIG. 5). In addition to the compounds in FIG. 2A, additional candidate biomarkers of microbial metabolism of levodopa were detected using untargeted metabolomics (FIG. 6).

TABLE 4
Summary - LC-MS Analysis of Human Plasma Samples

| | | 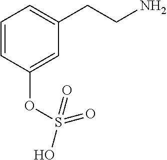 [SMILES: NCCc1cccc(OS(=O)(=O)O)c1] | 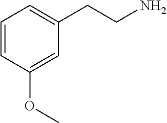 | 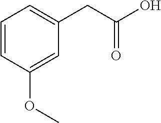 |
|---|---|---|---|---|
| | | C₈H₁₁NO₄S | C₉H₁₃NO | C₉H₁₀O₃ |
| Donor | Status | m/z: 218.0465 RT: 3.80 | m/z: 152.1060 RT: 7.90 | m/z: 167.0691 RT: 5.60 |
| 1 | PD | nan | 188317 | 104190 |
| 2 | PD | 2006 | 214238 | 288079 |
| 3 | PD | 7179 | 285199 | 127461 |
| 4 | PD | 11401 | nan | nan |
| 5 | PD | 248584 | nan | nan |
| 6 | PD | 70663 | nan | nan |
| 7 | PD | 10295 | nan | nan |
| 8 | PD | 95444 | nan | nan |
| 9 | PD | 143146 | nan | nan |
| 10 | PD | 22412 | nan | nan |
| 11 | HC | 10503 | nan | nan |
| 12 | HC | 4822 | nan | nan |
| 13 | HC | 30175 | nan | nan |
| 14 | HC | 7876 | nan | nan |
| 15 | HC | 1515 | nan | nan |
| 16 | HC | 25941 | nan | nan |

### Example 9: Meta-Tyramine in Human Intestinal Samples

Using a high-resolution LC-MS metabolomics platform, human intestinal samples from Parkinson's disease patients on levodopa therapy (PD donors) vs. healthy controls (HC donors) were evaluated. These intestinal samples were from different regions of the gastrointestinal tract in 13 HC donors (59 HC samples total) and 10 PD donors (68 PD samples total).

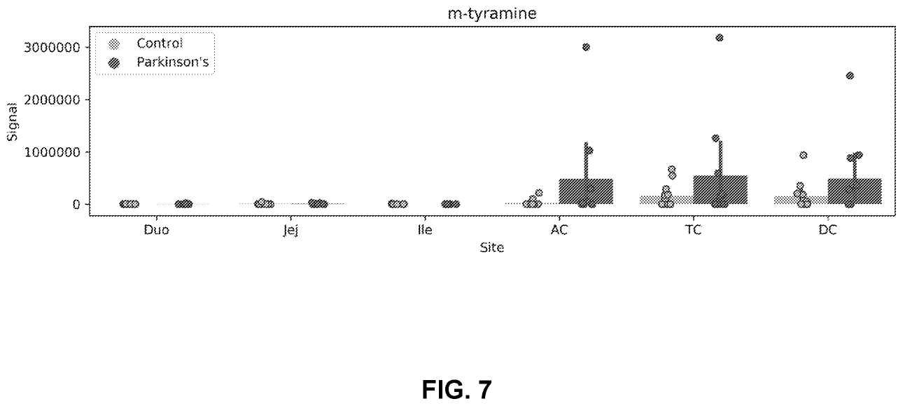

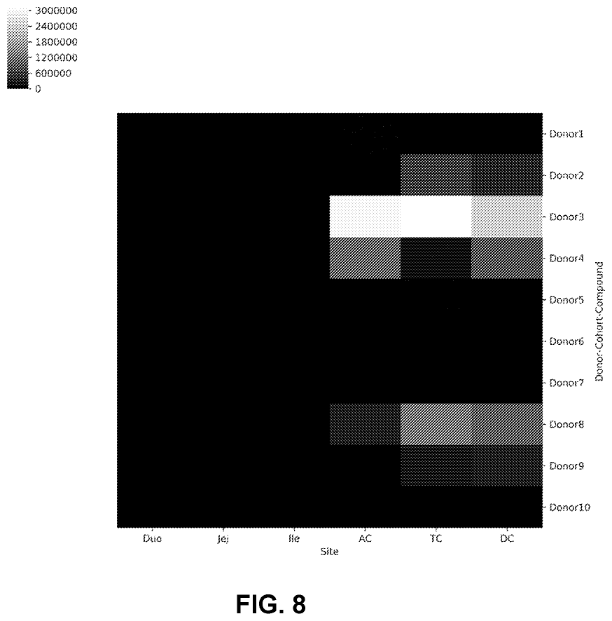

Briefly, samples were extracted and spun down using an appropriate solvent system and analyzed using a Vanquish™ UHPLC system-Q Exactive™ HF mass analyzer (Thermo Fisher Scientific). Separation was done using a reversed-phase column and a gradient of methanol (mobile phase B) in water (mobile phase A) over 15 min. Mass analysis was done at 120K resolution. Identity of meta-tyramine in each sample was confirmed by matching retention time, exact mass, and fragmentation pattern with authentic standards. The meta-tyramine was chromatographically resolved from the naturally dominant isomer of para-tyramine and confirmed using authentic standards. The relative signal for meta-tyramine in each region of the gastrointestinal tract was calculated for both the PD and HC cohorts (FIG. 7). Meta-tyramine showed regiospecific signals in the gastrointestinal tract of PD donors, with the highest signals in the lower intestine, including in the ascending colon, transverse colon, and descending colon. For the 10 PD donors, these signals were also represented as heat maps (FIG. 8).

1. A method of treatment, comprising administering a levodopa therapy comprising a tyrosine decarboxylase inhibitor to a Parkinson's disease patient who has an elevated level of meta-tyramine or a metabolic derivative thereof; or administering a levodopa therapy lacking a tyrosine decarboxylase inhibitor to a Parkinson's disease patient who has a normal or low level of meta-tyramine or a metabolic derivative thereof.

2. A method of treatment, comprising administering a levodopa therapy comprising a tyrosine decarboxylase inhibitor to a Parkinson's disease patient who has an elevated level of meta-tyramine or a metabolic derivative thereof.

3. A method of treating Parkinson's disease in a patient in need thereof, comprising:
(a) determining that the patient has an elevated level of meta-tyramine or a metabolic derivative thereof; and
(b) administering a levodopa therapy comprising a tyrosine decarboxylase inhibitor to the patient.

4. A method of treating Parkinson's disease in a patient in need thereof, comprising:
(a) determining that the patient has a normal or low level of meta-tyramine or a metabolic derivative thereof; and
(b) administering a levodopa therapy lacking a tyrosine decarboxylase inhibitor to the patient.

5. A method of providing a therapeutic regimen for treating Parkinson's disease in a patient in need thereof, comprising:
(a) determining that the patient has an elevated level of meta-tyramine or a metabolic derivative thereof; and
(b) providing a levodopa therapy comprising a tyrosine decarboxylase inhibitor to the patient.

6. A method of providing a therapeutic regimen for treating Parkinson's disease in a patient in need thereof, comprising:
(a) determining that the patient has a normal or low level of meta-tyramine or a metabolic derivative thereof; and
(b) providing a levodopa therapy lacking a tyrosine decarboxylase inhibitor to the patient.

7. The method of any one of claims 1 to 6, further comprising obtaining a biological sample from the patient, and determining the level of meta-tyramine or a metabolic derivative thereof in the sample.

8. A method of treating Parkinson's disease in a patient in need thereof, comprising:
(a) obtaining a biological sample from the patient;
(b) determining from the sample that the patient has an elevated level of meta-tyramine or a metabolic derivative thereof; and
(c) administering a levodopa therapy comprising a tyrosine decarboxylase inhibitor to the patient.

9. A method of treating Parkinson's disease in a patient in need thereof, comprising:
(a) obtaining a biological sample from the patient;
(b) determining from the sample that the patient has a normal or low level of meta-tyramine or a metabolic derivative thereof; and
(c) administering a levodopa therapy lacking a tyrosine decarboxylase inhibitor to the patient.

10. A method of identifying a suitable levodopa therapy for a Parkinson's disease patient, the method comprising:
(a) obtaining a biological sample from the patient;
(b) determining from the sample that the patient has an elevated level of meta-tyramine or a metabolic derivative thereof; and
(c) identifying a levodopa therapy comprising a tyrosine decarboxylase inhibitor as a suitable levodopa therapy for the patient.

11. A method of identifying a suitable levodopa therapy for a Parkinson's disease patient, the method comprising:
(a) obtaining a biological sample from the patient;
(b) determining from the sample that the patient has a normal or low level of meta-tyramine or a metabolic derivative thereof; and
(c) identifying a levodopa therapy lacking a tyrosine decarboxylase inhibitor as a suitable levodopa therapy for the patient.

12. The method of any one of claims 7 to 11, wherein the biological sample comprises a plasma sample, a urine sample, a stool sample, an intestinal sample, or a combination thereof.

13. The method of any one of claims 7 to 12, wherein the biological sample comprises a plasma sample and a urine sample.

14. The method of claim 12 or claim 13, wherein the plasma sample comprises peripheral blood plasma.

15. The method of any one of claims 7 to 12, wherein the biological sample comprises an intestinal sample from the duodenum, the jejunum, the ileum, the ascending colon, the descending colon, and/or the transverse colon.

16. The method of any one of claims 1 to 15, wherein the patient is receiving a levodopa therapy lacking a tyrosine decarboxylase inhibitor.

17. The method of claim 16, wherein the level of meta-tyramine or a metabolic derivative thereof is determined less than about 5 hours after the patient is administered a single dose of the levodopa therapy lacking a tyrosine decarboxylase inhibitor.

18. The method of claim 16 or claim 17, wherein the level of meta-tyramine or a metabolic derivative thereof is determined about 1 to about 3 hours after the patient is administered a single dose of the levodopa therapy lacking a tyrosine decarboxylase inhibitor.

19. The method of any one of claims 1 to 18, wherein the level of meta-tyramine or a metabolic derivative thereof is measured by metabolomics or enzyme-linked immunosorbent assay (ELISA).

20. The method of claim 19, wherein the metabolomics comprises liquid chromatography-mass spectrometry (LC-MS), gas-phase chromatography-mass spectrometry (GC-MS), or tandem mass spectrometry (MS-MS).

21. The method of claim 19 or claim 20, wherein the metabolomics comprises reversed-phase chromatography with positive ionization mode, reversed-phase chromatography with negative ionization mode, hydrophobic interaction liquid ion chromatography (HILIC) with positive ionization mode, hydrophobic interaction liquid ion chromatography (HILIC) with negative ionization mode, or a combination thereof.

22. The method of any one of claims 19 to 21, wherein the metabolomics comprises a combination of reversed-phase chromatography with positive ionization mode, reversed-phase chromatography with negative ionization mode, HILIC with positive ionization mode, and HILIC with negative ionization mode.

23. The method of any one of claims 1 to 22, wherein an elevated level of meta-tyramine or a metabolic derivative thereof in the patient is a level exceeding the level in a healthy subject naïve to levodopa; and wherein a normal or low level of meta-tyramine or a metabolic derivative thereof in the patient is a level equal to or below the level in a healthy subject naïve to levodopa.

24. The method of any one of claims 1 to 23, wherein an elevated level of meta-tyramine or a metabolic derivative thereof in the patient is a level exceeding 100 ng/mL; and wherein a normal or low level of meta-tyramine or a metabolic derivative thereof in the patient is a level equal to or below 100 ng/mL.

25. The method of any one of claim 1-3, 7, 8, or 12-24, wherein the levodopa is administered simultaneously with the tyrosine decarboxylase inhibitor.

26. The method of any one of claim 1-3, 7, 8, or 12-24, wherein the levodopa is administered sequentially with the tyrosine decarboxylase inhibitor.

27. The method of claim 25 or claim 26, wherein the levodopa therapy comprising a tyrosine decarboxylase inhibitor results in an increased level of circulating levodopa compared to the level of circulating levodopa prior to treatment.

28. The method of any one of claims 25 to 27, wherein the amount of levodopa administered in combination with the tyrosine decarboxylase inhibitor is reduced compared to the amount of levodopa administered in the absence of the tyrosine decarboxylase inhibitor.

29. The method of any one of claims 25 to 28, wherein the amount of levodopa administered in combination with the tyrosine decarboxylase inhibitor is reduced by at least 10% compared to the amount of levodopa administered in the absence of the tyrosine decarboxylase inhibitor.

30. The method of any one of claims 25 to 29, wherein the levodopa administered in combination with the tyrosine decarboxylase inhibitor is administered less frequently compared to the levodopa administered in the absence of the tyrosine decarboxylase inhibitor.

31. The method of any one of claims 25 to 30, wherein the levodopa administered in combination with the tyrosine decarboxylase inhibitor is administered at least 10% less frequently compared to the levodopa administered in the absence of the tyrosine decarboxylase inhibitor.

32. The method of any one of claims 25 to 31, wherein the treatment with levodopa in combination with the tyrosine decarboxylase inhibitor results in reduced systemic toxicity and/or improved tolerance compared to the treatment with levodopa in the absence of the tyrosine decarboxylase inhibitor.

33. The method of any one of claims 1 to 32, wherein the levodopa therapy further comprises a peripheral aromatic amino acid decarboxylase inhibitor.

34. The method of claim 33, wherein the peripheral aromatic amino acid decarboxylase inhibitor is carbidopa.

35. The method of any one of claims 1 to 34, wherein the tyrosine decarboxylase inhibitor is alpha-fluoromethyltyrosine (AFMT).

36. The method of any one of claims 1 to 34, wherein the tyrosine decarboxylase inhibitor is a compound chosen from the following compounds: 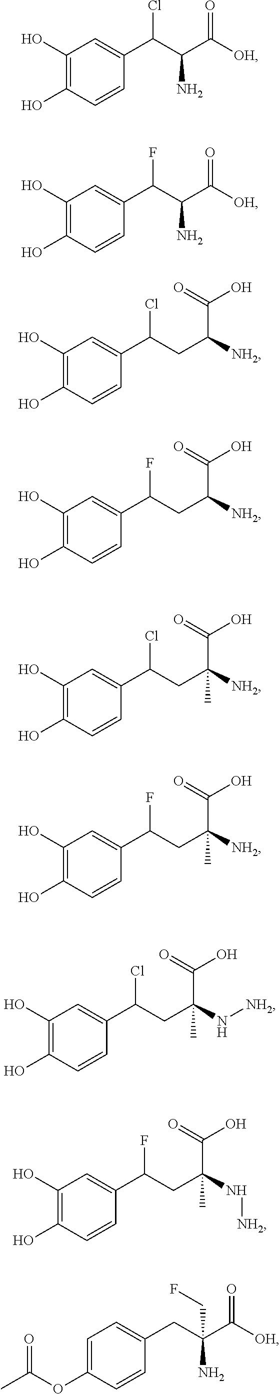 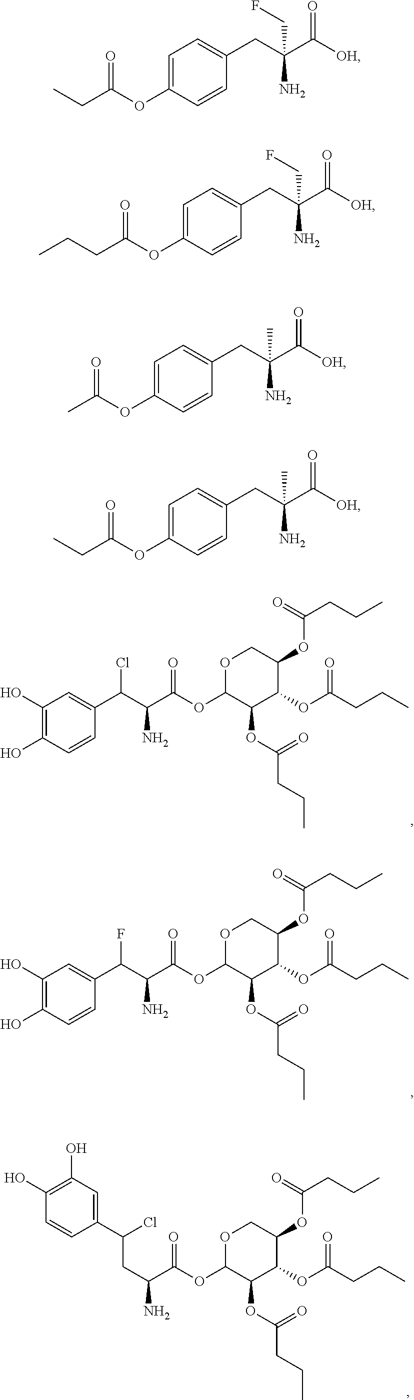 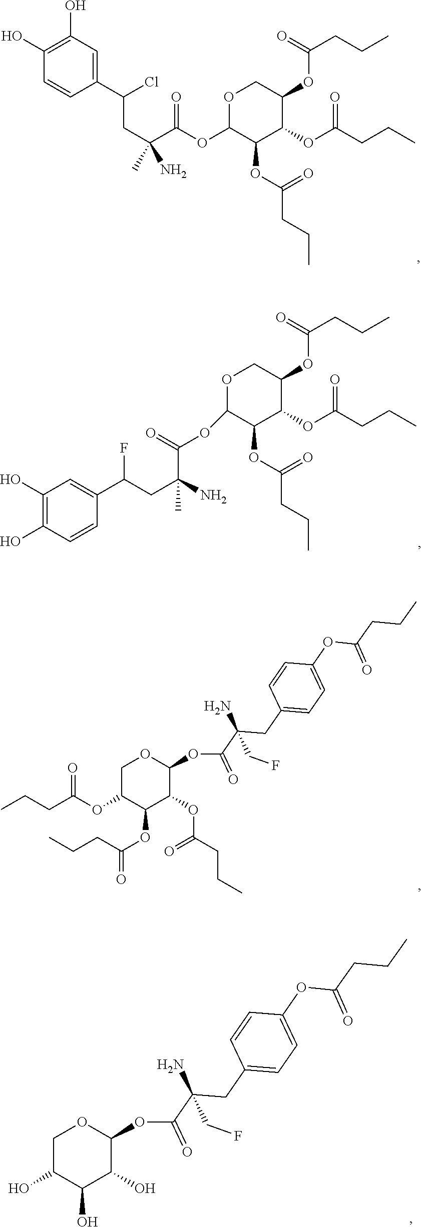 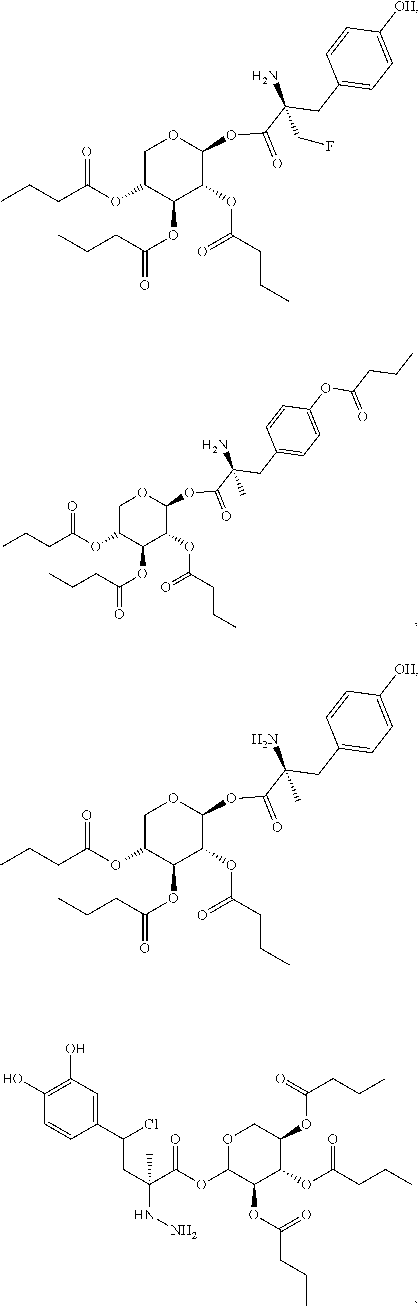 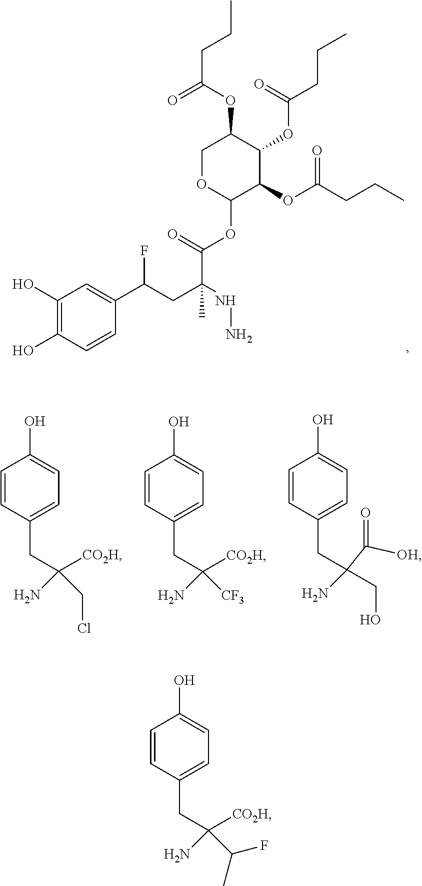 [SMILES: CCCC(=O)O[C@H]1[C@H](OC(=O)CCC)COC(OC(=O)[C@](C)(CC(F)c2ccc(O)c(O)c2)NN)[C@@H]1OC(=O)CCC ; NC(CCl)(Cc1ccc(O)cc1)C(=O)O ; NC(Cc1ccc(O)cc1)(C(=O)O)C(F)(F)F ; NC(CO)(Cc1ccc(O)cc1)C(=O)O ; CC(F)C(N)(Cc1ccc(O)cc1)C(=O)O]
and pharmaceutically acceptable salts thereof.

37. The method of any one of claims 1 to 34, wherein the tyrosine decarboxylase inhibitor is a compound chosen from the following compounds: 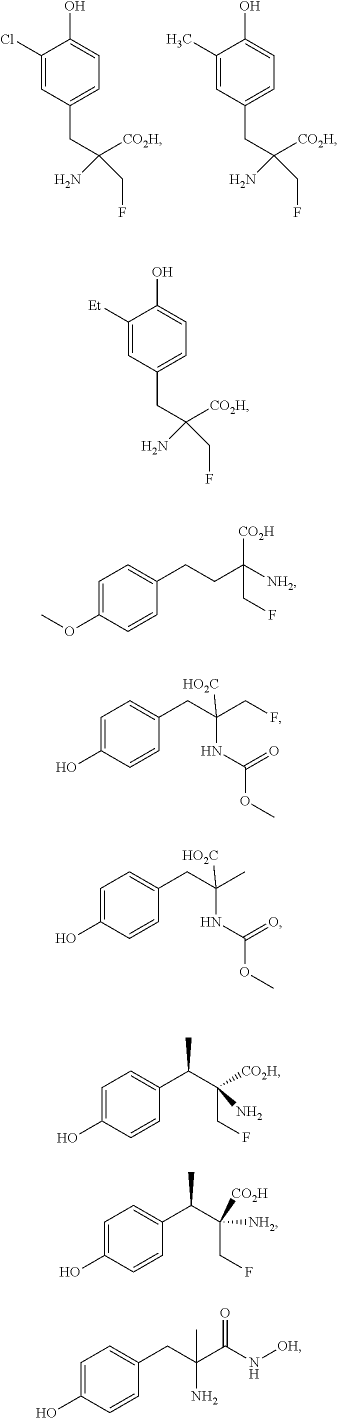 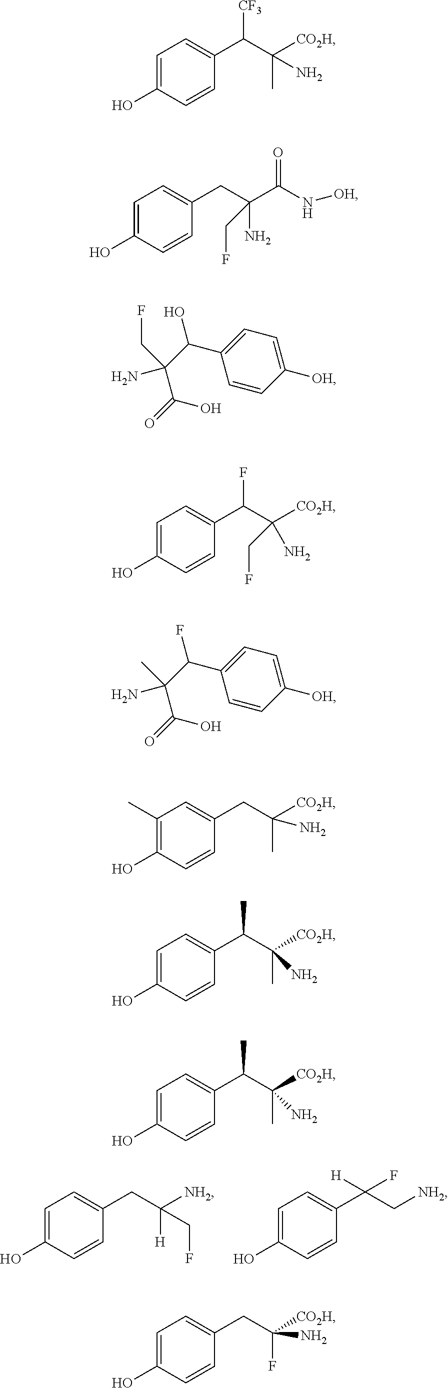 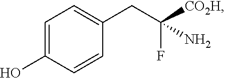 [SMILES: N[C@](F)(Cc1ccc(O)cc1)C(=O)O]
and pharmaceutically acceptable salts thereof.

38. The method of any one of claims 1 to 34, wherein the tyrosine decarboxylase inhibitor is a compound of formula (I): 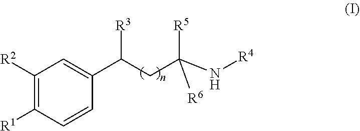
or a pharmaceutically acceptable salt thereof,
wherein
n is 0 or 1;
R¹ is H or —OR^{A}, wherein R^{A} is H, —C(O)C₁₋₆ alkyl, or an acylated sugar;
R² is H, halogen, amino, C₁₋₆ alkyl, or —OR^{A}, wherein R^{A} is H or an acylated sugar;
R³ is H, a halogen, —OH, or C₁₋₆ alkyl optionally substituted with one or more halogens;
R⁴ is H, —NH₂, —C(O)OCH₃, or an acylated sugar;
R⁵ is H, —C(O)OH, —C(O)OC₁₋₆ alkyl, —C(O)Oglycoside, —C(O)NHOH, or —C(O)O(acylated sugar); and
R⁶ is H, halogen, or optionally substituted C₁₋₆ alkyl;
provided that at least one R^{A} is present; or provided that R³ and/or R⁶ comprise a halogen.

39. The method of claim 38, wherein the tyrosine decarboxylase inhibitor is a compound of formula (I-a):

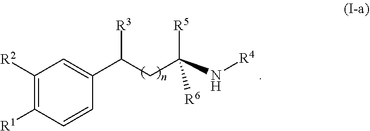

40. The method of claim 38 or claim 39, wherein
n is 0 or 1;
R¹ is H, —C(O)C₁₋₆alkyl, or —OR^{A}, wherein R^{A} is H or an acylated sugar;
R² is H, or —OR^{A}, wherein R^{A} is H or an acylated sugar;
R³ is H, or a halogen;
R⁴ is H, —NH₂, or an acylated sugar;
R⁵ is —C(O)OH, —C(O)OC₁₋₆ alkyl, —C(O)Oglycoside, or —C(O)O(acylated sugar); and
R⁶ is H or optionally substituted C₁₋₆ alkyl;
provided that at least one R^{A} is present; or provided that R³ and/or R⁶ comprise a halogen.

41. The method of any one of claims 38 to 40, wherein R¹ is —OR^{A}.

42. The method of any one of claims 38 to 41, wherein R² is H or —OR^{A}.

43. The method of any one of claims 38 to 42, wherein each R^{A} is H.

44. The method of claim 38 or claim 39, wherein R² is a halogen.

45. The method of any one of claims 38 to 44, wherein R³ is fluoro or chloro.

46. The method of any one of claims 38 to 44, wherein R³ is H.

47. The method of any one of claims 38 to 46, wherein R⁴ is H.

48. The method of any one of claims 38 to 46, wherein R⁴ is —NH₂.

49. The method of any one of claims 38 to 48, wherein R⁵ is —C(O)OH.

50. The method of any one of claims 38 to 48, wherein R⁵ is —C(O)Oacylated sugar.

51. The method of any one of claim 38, 39, or 41-48, wherein R⁵ is H.

52. The method of any one of claims 38 to 51, wherein R⁶ is H.

53. The method of any one of claims 38 to 51, wherein R⁶ is a C₁₋₆ alkyl.

54. The method of any one of claims 38 to 51, wherein R⁶ is a C₁₋₆ alkyl substituted with one, two, or three halogens.

55. The method of any one of claims 38 to 51, wherein R⁶ is a C₁₋₆ alkyl substituted with one, two, or three fluorine atoms.

56. The method of any one of claims 38 to 55, wherein n is 0.

57. The method of any one of claims 38 to 55, wherein n is 1.

58. The method of claim 38 or claim 39, wherein
n is 0;
R¹ is —OH;
R² is halogen;
R³ is H, a halogen, or —OH, C₁₋₆ alkyl optionally substituted with one or more halogens;
R⁴ is H, —NH₂, or an acylated sugar;
R⁵ is H, —C(O)OH, —C(O)OC₁₋₆ alkyl, —C(O)Oglycoside, —C(O)NHOH, or —C(O)O(acylated sugar); and
R⁶ is H or optionally substituted C₁₋₆ alkyl.

59. The method of any one of claim 38, 39, or 58, wherein
n is 0;
R¹ is —OH;
R² is halogen;
R³ is H;
R⁴ is H;
R⁵ is —C(O)OH; and
R⁶ is optionally substituted alkyl.

60. The method of any one of claims 1 to 59, wherein the meta-tyramine or a metabolic derivative thereof comprises meta-tyramine, 3-hydroxyphenylacetic acid, 3-hydroxyphenylacetaldehyde, 3-hydroxyphenylacetate methyl ester, 3-sulfooxyphenylacetic acid, 3-methoxyphenylacetic acid, 3-methoxyphenethylamine, 3-hydroxyphenylethanol, 3-hydroxymandelic acid, meta-octopamine, meta-tyramine-O-sulfate, and/or meta-tyramine-O-glucuronide.

61. The method of any one of claims 1 to 60, wherein the meta-tyramine or a metabolic derivative thereof comprises meta-tyramine, 3-hydroxyphenylacetic acid, 3-hydroxyphenylacetate methyl ester, 3-sulfooxyphenylacetic acid, 3-methoxyphenylacetic acid, 3-methoxyphenethylamine, and/or meta-tyramine-O-sulfate.

62. The method of any one of claims 1 to 61, wherein the meta-tyramine or a metabolic derivative thereof comprises 3-hydroxyphenylacetic acid, 3-hydroxyphenylacetate methyl ester, 3-sulfooxyphenylacetic acid, and/or meta-tyramine-O-sulfate.
